(19) United States
(12) Patent Application Publication
(10) Pub. No.: US 2025/0164768 A1
(43) Pub. Date: May 22, 2025

(54) SYSTEMS AND METHODS FOR LIGHT MANIPULATION

(71) Applicant: ENSPECTRA HEALTH, INC., Mountain View, CA (US)

(72) Inventor: Gabriel Sanchez, Menlo Park, CA (US)

(21) Appl. No.: 18/671,195

(22) Filed: May 22, 2024

Related U.S. Application Data
(63) Continuation of application No. PCT/US2022/051585, filed on Dec. 1, 2022.
(60) Provisional application No. 63/285,422, filed on Dec. 2, 2021.

(51) Int. Cl. G02B 21/00

(52) U.S. Cl. CPC G02B 21/0032

(57) ABSTRACT

The present disclosure provides systems, devices, and methods for imaging and imaging subject. The systems and devices may include one or more light filtering devices. The light filtering devices may include a chamber having an input and an output. The chamber may be configured to receive a beam of light. The chamber may comprise a plurality of reflectors configured to direct the light received in an optical path from the input to the output via reflectance of the light between one or more reflectors of the plurality of reflectors. The chamber may be configured to reject out of focus light along the optical path.

### CROSS-REFERENCE TO RELATED APPLICATIONS

This application is a continuation of International Application No. PCT/US2022/051585, filed Dec. 1, 2022, which claims the benefit of U.S. Provisional Application No. 63/285,422, filed Dec. 2, 2021, which is incorporated herein in its entirety.

### GOVERNMENT INTEREST STATEMENT

The invention(s) was made with U.S. Government support under Small Business innovation Research (SBIR) grant number 2R44CA221591-02A1 awarded by the Department of Health and Human Services, National Institutes of Health, National Cancer Institute. The U.S. Government has certain rights in the invention(s).

### BACKGROUND

Confocal microscopy imaging uses excitation light directed to tissue, and collects resulting reflected, transmitted or fluoresced light. Confocal microscopy imaging may create a high resolution image by passing the collected light through a pinhole (or a single mode fiber) to reject out of focus light. A focusing lens arrangement may be used to focus the collected light to a plane or spot that is optically congruent with the objective focus to the imaging plane in the tissue. The pinhole is used as a spatial filter where background or out of focus light is physically rejected or blocked while in focus light passes through the pinhole. Accurate alignment of the focused light is required on the x, y and z axes where the z axis is in the direction of the focused light beam. The pinhole and spot size depend on the selected resolution. The pinhole and alignment of the pinhole are on approximately the same scale as the resolution. In tissue imaging using reflectance confocal microscopy, a focused spot of light may be 1-10 microns and can be directed through a pinhole approximately 1-10 microns. Accordingly, reflectance confocal microscopy imaging may use a small spot of focused light to be centered or aligned on a very small opening. The focusing element tolerances of the small magnitude can be difficult to achieve, particularly in a small handheld device where it is easy to lose alignment with any instability and/or thermal expansion of the imaging structures during use.

### SUMMARY

Provided herein are improved system and methods for confocal microscopy imaging. Further provided herein are methods and apparatuses in a portable or handheld optical device. Further provided herein are methods and apparatuses for imaging tissue.

In an aspect, the present disclosure provides a device, comprising: a probe configured to (i) direct a beam of light from a light source to an imaging subject and (ii) collect light from the imaging subject upon the beam of light contacting the imaging subject; and a light filtering device in optical communication with the probe, wherein the light filtering device comprises (a) a chamber having (i) an input configured to receive the light collected by the probe and (ii) an output configured to transmit the light away from the chamber and (b) a plurality of reflectors disposed in the chamber, wherein the plurality of reflectors is configured to direct the light received, in an optical path from the input to the output via reflectance of the light between reflectors of the plurality of reflectors, wherein the chamber is configured to reject out of focus light along the optical path.

In an embodiment, a cross sectional diameter of the output is greater than or equal to about 0.1 times a cross sectional diameter of the input.

In an embodiment, the chamber has a longest internal linear dimension, and wherein the optical path has a path length that is at least 3 times a length of the longest linear dimension.

In an embodiment, the optical path has a pathlength greater than or equal to 0.25 meters, 0.5 meters, or 1 meter.

In an embodiment, at least one reflector of the plurality of reflectors is a retroreflector.

In an embodiment, at least one reflector of the plurality of reflectors is a mirror.

In an embodiment, one reflector of the plurality of reflectors is a mirror and another reflector of the plurality of reflectors is a retroreflector.

In an embodiment, the output is configured to direct a focused portion of the light to a collector.

In an embodiment, the device comprises the collector.

In an embodiment, the light filtering device is coupled to a fiber optic, and wherein the fiber optic is configured to provide the light to the collector.

In an embodiment, the plurality of reflectors includes a first reflector, wherein the plurality of reflectors are arranged to direct a focused portion of the light in an optical path between the reflectors, and wherein the optical path returns to the first reflector a plurality of times before reaching the output.

In an embodiment, the plurality of reflectors comprises a first reflector at a first location within the chamber and a second reflector at a second location within the chamber, which first reflector is configured to direct the light to the second reflector.

In an embodiment, the optical path has a path length, wherein the first reflector and the second reflector are separated by a distance, and wherein the pathlength is at least five times the distance.

In an embodiment, the pathlength that the light traverses is from five times to thirty times the distance.

In an embodiment, the pathlength is greater than or equal to about 0.5 meters.

In an embodiment, the distance is less than or equal to about 15 centimeters.

In an embodiment, the first reflector and the second reflector are spaced apart along an axis parallel to a length of the chamber.

In an embodiment, the device further comprises a scanning unit disposed between the light source and the probe, wherein the scanning unit is configured to scan the beam of light across the imaging subject.

In an embodiment, the device further comprises a focusing unit disposed between the light source and the probe, wherein the focusing unit is configured to scan a focal point of the beam of light within the imaging subject.

In an embodiment, the probe is configured to contact or penetrate the imaging subject.

In an embodiment, the probe comprises an objective configured to collimate the light.

In an embodiment, the device further comprises an alignment unit disposed between the probe and the light filtering device, wherein the alignment unit is configured to (i) direct the light to the light filtering device and (ii) adjust an angle of entry of the light into the chamber.

In an embodiment, the device further comprises a beam splitter disposed between the probe and the light filtering device, wherein the beam splitter is configured to (i) split the light to generate a split light and (ii) direct at least a portion of the split light to the light filtering unit.

In an embodiment, the device is configured for confocal imaging.

In an embodiment, the device is configured for tandem confocal imaging and multiphoton imaging.

In an embodiment, the device is portable.

In another aspect, the present disclosure provides a device, comprising: a chamber, wherein the chamber is configured to receive a beam of light; and a plurality of reflectors comprising a first reflector and a second reflector, wherein the first reflector is disposed in the chamber; and the second reflector is disposed in the chamber, wherein the first reflector is configured to direct the beam of light from the first reflector to the second reflector, wherein the second reflector is configured to direct the beam of light from the second reflector to the first reflector at least once before the first reflector, the second reflector, or another reflector of the plurality of reflectors directs the beam of light out of the chamber.

In an embodiment, the chamber is configured to filter out unfocused light from the beam of light.

In an embodiment, the first reflector or the second reflector is a retroreflector.

In an embodiment, the retroreflector is configured for total internal reflection.

In an embodiment, at least one of the first reflector or the second reflector is a mirror.

In an embodiment, the mirror comprises a dielectric coating.

In an embodiment, the dielectric coating is configured to direct the portion of the beam of light having a wavelength from about 700 nanometers to 900 nanometers.

In an embodiment, the chamber further comprises an input and an output, wherein the input is configured to receive the signal, and wherein the output is configured to direct the signal to a collection unit.

In an embodiment, the collection unit is disposed adjacent to the first location or the second location, wherein the collection unit is configured to collect the beam of light.

In an embodiment, the first reflector and the second reflector are spaced apart along an axis parallel to a length of the chamber.

In an embodiment, a distance in the chamber separates the first reflector and the second reflector and a pathlength that the beam of light traverses within the chamber is at least three times the distance separating the first reflector and the second reflector.

In an embodiment, the pathlength the beam of light traverses is at least five times the distance separating the first reflector and the second reflector.

In an embodiment, the pathlength is from five times to thirty times the distance separating the first reflector and the second reflector.

In an embodiment, the distance between the first reflector and the second reflector is less than or equal to about 15 centimeters.

In an embodiment, the distance between the first reflector and the second reflector is less than or equal to about 10 centimeters.

In an embodiment, the pathlength is greater than or equal to about 0.5 meters.

In an embodiment, the pathlength is greater than or equal to about 1 meter.

In an embodiment, the first reflector or the second reflector has a reflective surface with a dimension from about 0.5 centimeters to 2 centimeters.

In an embodiment, the device further comprises a probe configured to (i) provide a beam of light from a light source to an imaging subject and (ii) collect light from the imaging subject upon the beam of light contacting the imaging subject; wherein the probe is in optical communication with the chamber.

In an embodiment, the first reflector or the second reflector comprises a plurality of reflective elements.

In an aspect, the present disclosure provides a system for filtering light, comprising: a light source for generating a beam of light; a light filtering device in optical communication with the light source, wherein the filtering device comprises: (i) a chamber configured to receive the beam of light from the light source and the chamber having an input configured to receive the light from the probe and an output configured to transmit the light away from the chamber; and (ii) a plurality of reflectors disposed in the chamber, and configured to direct the light received, in an optical path from the input to the output via reflectance of the light between reflectors of the plurality of reflectors, wherein the chamber is configured to reject out of focus light along the optical path; and one or more computer processors operatively coupled to the light source and the light filtering device, wherein the one or more computer processors are individually or collectively programed to process the beam of light transmitted away from the chamber at the output, to generate an image.

In an embodiment, the system further comprises a beam de-expander unit positioned between the beam of light and the light filtering device.

In an embodiment, the beam de-expander unit comprises a chamber of the beam de-expander unit comprising a first lens and a second lens, wherein the first lens is positioned at a first end of the chamber of the beam de-expander unit and the second lens is positioned on a second end of the chamber of the beam de-expander unit, wherein the first end of the chamber of the beam de-expander unit and the second end of the chamber of the beam de-expander unit are opposite.

In an embodiment, the chamber of the beam de-expander unit comprises a deformable wall.

In an embodiment, the beam de-expander unit comprises a tuning structure.

In an embodiment, the tuning structure is configured to adjust a distance between the first lens and the second lens.

In an embodiment, the system further comprises an imaging device comprising the light filtering device.

In an embodiment, a cross sectional diameter of the output is greater than or equal to about 0.1 times a cross sectional diameter of the input.

In an embodiment, the chamber has a longest internal linear dimension, wherein the path has a path length that is at least 3 times as long as the longest linear dimension.

In an embodiment, the imaging device comprises a probe disposed in an optical pathway between the light source and the light filtering device, wherein the probe is configured to (i) direct the beam of light to a imaging subject, (ii) collect a light from a imaging subject upon the beam of light contacting the imaging subject, and (iii) direct the signal to the light filtering device.

In an embodiment, the probe is configured to contact or penetrate the imaging subject.

In an embodiment, the probe comprises an objective configured to collimate the signal. In an embodiment, the imaging device is a handheld device.

In an embodiment, the imaging device further comprises one or more members selected from the group consisting of a focusing unit, a scanning unit, an alignment unit, an objective, a beam splitter, a frequency multiplier, and any combination thereof.

In an embodiment, the system further comprises a focusing unit disposed in an optical pathway between the light source and the light filtering device, wherein the focusing unit is configured to vary a focal point of the beam of light.

In an embodiment, the system further comprises a scanning unit disposed in an optical pathway between the light source and the light filtering device, wherein the scanning unit is configured to scan the beam of light in at least one dimension.

In an embodiment, the system further comprises an alignment unit disposed in an optical pathway between the light source and the light filtering device, wherein the alignment unit is configured to (i) direct the beam of light to the light filtering device and (ii) adjust an angle of entry of the beam of light into the chamber.

In an embodiment, the alignment unit comprises a mirror.

In an embodiment, the system further comprises a beam splitter disposed in an optical pathway between the light source and the light filtering device, wherein the beam splitter is configured to (i) split the beam of light to generate a split beam of light and (ii) direct at least a portion of the split beam of light to the light filtering unit.

In an embodiment, the plurality of reflectors include a first reflector, wherein the plurality of reflectors are arranged to direct a focused portion of the light in a path between the reflectors, and wherein the path returns to the first reflector a plurality of times before reaching the output.

In an embodiment, the plurality of reflectors comprises a first reflector disposed in a first location in the chamber and configured to direct the light to a second location in the chamber, and (c) a second reflector disposed in the second location and configured to direct the light to the first reflector.

In an embodiment, a distance in the chamber separates the first reflector and the second reflector, and wherein a pathlength that the light traverses between the first location and the second location is at least three times the distance separating the first reflector and the second reflector.

In an embodiment, the pathlength the beam of light traverses is at least five times the distance separating the first reflector and the second reflector.

In an embodiment, the pathlength the beam of light traverses is from about five times to thirty times the distance separating the first reflector and the second reflector.

In an embodiment, the distance between the first reflector and the second reflector is less than or equal to about 15 centimeters.

In an embodiment, the pathlength is greater than or equal to about 0.5 meters.

In an embodiment, at least one of the first reflector or the second reflector is a mirror.

In an embodiment, the first reflector, the second reflector, or both the first reflector and the second reflector comprises a retroreflector.

In an embodiment, the system further comprises a fiber bundle comprising a first end coupled to an output of the chamber and a second end coupled to a collection unit, wherein the fiber bundle is configured to collect the beam of light subsequent to the beam of light passing through the chamber and direct the beam of light to the collection unit.

In an embodiment, the first end of fiber bundle comprises a linear array of fibers.

In an embodiment, a diameter of the fiber bundle is from about 1 millimeter to about 5 millimeters.

In an embodiment, the system further comprises a confocal imaging arrangement, and wherein the light filtering device is part of the confocal imaging arrangement.

In an embodiment, the confocal imaging arrangement does not include an emission pinhole for light filtering.

In an embodiment, the system further comprises a multiphoton imaging arrangement.

In an embodiment, the system is configured for tandem confocal imaging and multiphoton imaging.

In an embodiment, the system is configured to generate three-dimensional images of an imaging subject.

In an embodiment, the system is portable.

In an aspect, the present disclosure provides a method for filtering light, comprising: (a) providing a light filtering device comprising (i) a chamber having a plurality of reflectors arranged to reflect a light beam between plurality of reflectors to create an optical path; (b) directing a beam of light into the chamber; and (c) directing a focused portion of the beam of light in the optical path from the input, between the reflectors and to the output while removing unfocused light from the optical path.

In an embodiment, the method further comprises, prior to (b), transmitting the beam of light from a light source to an imaging subject and collecting light from the imaging subject upon the beam of light contacting the imaging subject.

In an embodiment, the method further comprises using a focusing unit disposed in an optical pathway between the light source and the imaging subject to vary a focal point of the beam of light within the imaging subject.

In an embodiment, the method further comprises using a scanning unit disposed in an optical pathway between the light source and the imaging subject to scan the beam of light across the imaging subject in at least one dimension.

In an embodiment, the method further comprises using a beam splitter disposed in an optical pathway between the imaging subject and the light filtering device to (i) split the light to generate a split light and (ii) direct at least a portion of the split light to the light filtering device.

In an embodiment, the method further comprises using a probe disposed in an optical pathway between the light source and the imaging subject to direct the beam of light to the imaging subject and collect the light generated from the imaging subject.

In an embodiment, the method further comprises, subsequent to directing a focused portion of the beam of light to the output of the chamber, processing the light to generate an image of the imaging subject.

In an embodiment, the image is a three-dimensional image.

In an embodiment, the method further comprises using the light for confocal imaging.

In an embodiment, the method further comprises, prior to (b), using an alignment unit to (i) align the beam of light with the chamber and (ii) adjust an angle of entry of the beam of light into the chamber.

In an embodiment, the light filtering device is provided in an imaging device.

In an embodiment, the imaging device is handheld.

In an embodiment, the imaging device further comprises a confocal imaging arrangement comprising the light filtering device.

In an embodiment, the imaging device further comprises a multiphoton imaging arrangement.

In an embodiment, the method further comprises using the confocal imaging arrangement and the multiphoton imaging arrangement for tandem confocal and multiphoton imaging.

In an embodiment, the imaging device comprises an objective that collimates the beam of light.

In an embodiment, a pathlength of the optical path that the beam of light traverses within the chamber is at least 0.25 M.

In an embodiment, the beam of light is a collimated beam of light.

In an aspect, the present disclosure provides a method for imaging an imaging subject, comprising: (a) providing a probe in optical communication with (i) a light filtering device comprising a chamber and (ii) the imaging subject; (b) using the probe to provide a beam of light to the imaging subject and collect a resulting return beam of light from the imaging subject; (c) directing the return beam of light from the imaging subject to the chamber of the light filtering device; (d) repeatedly directing the beam of light from a first reflector to a second reflector within the chamber of the light filtering device such that a pathlength that the beam of light traverses between the first reflector and the second reflector is at least three times a distance separating the first reflector and the second reflector; and (e) processing the beam of light to generate an image of the imaging subject.

In an embodiment, the method further comprises, prior to (b), transmitting the beam of light from a light source to an imaging subject via the probe and collecting light from the imaging subject upon the beam of light contacting the imaging subject.

In an embodiment, the method further comprises, prior to (c), directing the return beam of light to a beam de-expander unit. In an embodiment, the method further comprises tuning the beam de expander after directing the return beam of light to a beam de-expander unit.

In an embodiment, the beam de-expander unit comprises a chamber of the beam de-expander unit comprising a first lens and a second lens, wherein the first lens is positioned on a first end of the chamber of the beam de-expander unit and the second lens is positioned on a second end of the chamber of the beam de-expander unit, wherein the first end of the chamber of the beam de-expander unit and the second end of the chamber of the beam de-expander unit are opposite.

In an embodiment, the chamber of the beam de-expander unit comprises a deformable wall.

In an embodiment, the beam de-expander unit further comprises a tuning structure.

In an embodiment, the tuning structure is configured to adjust a distance between the first lens and the second lens.

In an embodiment, the method further comprises, subsequent to (c), processing the light to generate an image of the imaging subject.

In an embodiment, the method further comprises using the light for confocal imaging.

In an embodiment, the method further comprises using the light for multiphoton imaging.

In an embodiment, the method further comprises using the light for tandem confocal and multiphoton imaging.

In an embodiment, the pathlength the beam of light traverses is at least five times the distance separating the first location and the second location.

In an embodiment, the pathlength is from five times to thirty times the distance separating the first reflector and the reflector.

In an embodiment, the pathlength is greater than or equal to about 0.5 meters.

In an embodiment, the distance is less than or equal to about 15 centimeters.

In an aspect, the present disclosure provides an imaging device, comprising: a light source configured to provide a beam of light to an imaging subject; a probe in optical communication with the light source, wherein the probe is configured to direct the beam of light from the light source to the imaging subject; a light filtering device in optical communication with the probe, wherein the light filtering device comprises a chamber comprising a first reflector and a second reflector wherein the light filtering device is configured (i) to receive the beam of light from the probe and (ii) such that a pathlength that the beam of light traverses between the first reflector and the second reflector is at least three times a distance separating the first reflector and the second reflector; and a collection unit in optical communication with the light filtering device, wherein the collection unit is configured to collect the beam of light from the light filtering device, wherein the beam of light is usable to generate an image of the imaging subject.

In an embodiment, the method further comprises a beam de-expander unit positioned between the beam of light and the light filtering device.

In an embodiment, the beam de-expander unit comprises a chamber of the beam de-expander unit comprising a first lens and a second lens, wherein the first lens is positioned on a first end of the chamber of the beam de-expander unit and the second lens is positioned on a second end of the chamber of the beam de-expander unit, wherein the first end of the chamber of the beam de-expander unit and the second end of the chamber of the beam de-expander unit are opposite.

In an embodiment, the chamber of the beam de-expander unit comprises a deformable wall.

In an embodiment, the beam de-expander unit comprises a tuning structure.

In an embodiment, the tuning structure is configured to adjust a distance between the first lens and the second lens.

In an embodiment, the pathlength the beam of light traverses is at least five times the distance separating the first reflector and the second reflector.

In an embodiment, the pathlength is from five times to thirty times the distance separating the first reflector and the second reflector.

In an embodiment, the pathlength is greater than or equal to about 0.5 meters.

In an embodiment, the distance is less than or equal to about 15 centimeters.

In an aspect, the present disclosure provides a system, comprising: a probe configured to (i) provide a beam of light from a light source to an imaging subject and (ii) collect light from the imaging subject upon the beam of light contacting the imaging subject; and a light filtering device in optical communication with the probe, wherein the light filtering device comprises: (i) a chamber comprising an input and an output, the chamber configured to receive the light from the imaging subject at the input, and (ii) a plurality of reflectors positioned within the chamber, including a first reflector, wherein reflectors of the plurality of reflectors are arranged to direct a focused portion of the light in a path between the reflectors, and wherein the path returns to the first reflector a plurality of times before reaching the output.

In an aspect, the present disclosure provides a system, comprising: a probe configured to (i) provide a beam of light from a light source to an imaging subject and (ii) collect light from the imaging subject upon the beam of light contacting the imaging subject; and a light filtering device in optical communication with the probe, wherein the light filtering device comprises (a) a chamber having: (i) an input configured to receive the light from the probe and (ii) an output configured to transmit the light away from the chamber, and (b) a plurality of reflectors disposed in the chamber, and configured to direct the light received from the input to the output via reflectance of the light between reflectors of the plurality of reflectors, wherein a pathlength that the light traverses, via reflectance, from the input to the output via the reflectors, is at least 0.25 meters.

In an aspect, the present disclosure provides a device, comprising: a chamber that provides an optical path leading from an input to an output of the chamber, wherein: (i) the optical path is configured to direct light from the input to the output, and (ii) the chamber is configured to reject out of focus light along the optical path, wherein a cross sectional diameter of the output is greater than or equal to about 0.1 times a cross sectional diameter of the input.

In an aspect, the present disclosure provides a device, comprising: a chamber that provides an optical path leading from an input to an output of the chamber, wherein: (i) the optical path is configured to direct light from the input to the output, (ii) the chamber is configured to reject out of focus light along the optical path, wherein the chamber has a longest internal linear dimension wherein the optical path has path length that is at least 3 times a length of the longest linear dimension.

In an aspect, the present disclosure provides a device, comprising:
a chamber that provides an optical path leading from an input to an output of the chamber, wherein: (i) the optical path has a path length and is configured to direct light from the input to the output, and (ii) the chamber is configured to reject out of focus light along the optical path, wherein the chamber has a longest internal linear dimension, wherein the path length is at least 3 times as long as the longest linear dimension.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A and 1B schematically illustrate example confocal imaging arrangements;

FIG. 1A shows an example confocal imaging arrangement comprising a probe integrated with a light filtering device;

FIG. 1B shows an example confocal imaging arrangement including a probe connected to a light filtering device;

FIGS. 2A-2C schematically illustrate example light filtering devices and resulting paths of a beam of light;

FIG. 2A schematically illustrates an example light filtering device including a mirror and retroreflector;

FIG. 2B schematically illustrates an example light filtering device including two mirrors;

FIG. 2C schematically illustrates an example light filtering device including two retroreflectors;

FIGS. 3A and 3B schematically illustrate an optical fiber bundle of a collector;

FIG. 3A shows an example of a linear arrangement of an optical fiber bundle of a collector;

FIG. 3B shows an example of a circularly arranged optical fiber bundle of a collector;

FIGS. 4A and 4B schematically illustrate an example light path through a light filtering device;

FIG. 4A shows an example of a folded light path;

FIG. 4B shows an example of an effective optical length of the light path of FIG. 4A;

FIG. 5 shows example optical elements, including focusing units, usable for scanning an imaging subject;

FIG. 6 shows another example of optical elements, including focusing units, usable for scanning an imaging subject;

FIG. 7 schematically illustrates an example handheld device including optical elements for scanning an imaging subject;

FIG. 8 shows an example of a light path between a mirror and retroreflector;

FIG. 9 shows examples of optical elements of a plurality of reflection arrangements and a schematic light path;

FIGS. 10A-10D show example images generated from scanned in-vivo depth profiles;

FIG. 10A shows an example two photon image;

FIG. 10B shows an example second harmonic generation image;

FIG. 10C shows an example reflectance confocal microscopy image;

FIG. 10D shows an example three channel averaged image;

FIGS. 11A and 11B schematically illustrate an example support system;

FIG. 12 shows an example of a portable imaging system, including a handheld device coupled to a support system;

FIG. 13 shows an example portable support system for imaging;

FIG. 14 shows an example of an imaging system, including a handheld device, being used to image a subject;

FIG. 15 shows a computer system that is programmed or otherwise configured to implement methods provided herein;

FIG. 16 schematically illustrates an example light path through a beam de-expander;

FIGS. 17A-17D schematically illustrate an example of a beam de-expander with a chamber;

FIG. 17A schematically shows the beam de-expander arranged in an imaging system;

FIG. 17B shows the beam de-expander with an actuating element to deform the chamber;

FIG. 17C illustrates the beam de-expander and actuating element, with the beam de-expander chamber un-deformed;

FIG. 17D illustrates the beam de-expander with the chamber deformed;

FIGS. 18A-18C schematically illustrate an example of a beam de-expander with a chamber;

FIG. 18A illustrates the beam de-expander with an tuning element to deform the chamber;

FIG. 18B illustrates the beam de-expander with the chamber undeformed; and

FIG. 18C illustrates the beam de-expander with the chamber deformed.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to an animal, such as a mammal. A subject may be a human or non-human mammal. A subject may be a plant. A subject may be afflicted with a disease or suspected of being afflicted with or having a disease. The subject may not be suspected of being afflicted with or having the disease. The subject may be symptomatic. Alternatively, the subject may be asymptomatic. In some cases, the subject may be treated to alleviate the symptoms of the disease or cure the subject of the disease. A subject may be a patient undergoing treatment by a healthcare provider, such as a doctor.

The term “tissue characteristic” as used herein generally refers to a state of a tissue. Examples of a tissue characteristic include, but are not limited to a disease, an abnormality, a normality, a condition, a tissue hydration state, a tissue structure state, a health state of tissue, or a beneficial state. A characteristic can be a pathology. A characteristic can be benign (e.g., information about a healthy tissue). A tissue characteristic can comprise one or more features that can aid in tissue classification or diagnosis. A tissue characteristic may be eczema, dermatitis, psoriasis, lichen planus, bullous pemphigoid, vasculitis, granuloma annulare, Verruca vulgaris, seborrhoeic keratosis, basal cell carcinoma, actinic keratosis, squamous cell carcinoma in situ (e.g., an intraepidermal carcinoma), squamous cell carcinoma, cysts, lentigo, melanocytic naevus, melanoma, dermatofibroma, scabies, fungal infection, bacterial infection, burns, wounds, and the like, or any combination thereof.

The term “feature,” as used herein, generally refers to an aspect of a tissue or other body part that is indicative of a given tissue characteristic or multiple tissue characteristics. Examples of features include, but are not limited to a property; physiology; anatomy; composition; histology; function; treatment; size; geometry; regularity; irregularity; optical property; chemical property; mechanical property or other property; color; vascularity; appearance; structural element; quality; age of a tissue of a subject; data corresponding to a tissue characteristic; spongiosis in acute eczema with associated lymphocyte exocytosis; acanthosis in chronic eczema; parakeratosis and/or perivascular lymphohistiocytic infiltrate; excoriation and/or signs of rubbing (e.g., irregular acanthosis and perpendicular orientation of collagen in dermal papillae) in chronic cases (e.g., lichen simplex); hyperkeratosis (e.g., parakeratosis), orthokeratosis; neutrophils in stratum corneum and squamous cell layer; hypogranulosis; epidermis is thin over dermal papillae; regular acanthosis, clubbed rete ridges; relatively little spongiosis; dilated capillaries in dermal papillae; perivascular lymphohistiocytic infiltrate; orthokeratosis; hypergranulosis; irregular acanthosis with saw-toothed rete ridges; colloid bodies in lower epidermis and upper dermis; liquefaction degeneration of the basal layer; lichenoid lymphohistiocytic infiltrate in upper dermis (e.g., interface dermatitis) and/or the epidermis; melanin incontinence; subepidermal blister; viable roof over new blister, necrotic over an old blister; variable perivascular infiltrate (e.g., lymphocytes, histiocytes, eosinophils); pre-bullous lesions may show spongiosis with eosinophil exocytosis (e.g., eosinophilic spongiosis); vessel wall damage (e.g., necrosis, hyalinisation, fibrin); invasion of inflammatory cells into vessel walls; red cell extravasation; nuclear dust from leucocytoclasia of neutrophils; ischaemic necrosis of the epidermis; normal epidermis; central foci of dermal collagen degeneration (e.g., necrobiosis), mucin accumulation; palisading of histiocytes; multinucleate giant cells; single-filing of inflammatory cells between collagen bundles (e.g., ‘busy’ dermis); hyperkeratosis, papillomatosis, acanthosis; basaloid keratinocytes; horn cysts; abundant melanin in basal layer and/or throughout epidermis; sharp demarcation of base of epidermal hyperplasia; location; cohesive nests of basaloid tumor cells (e.g., sometimes with a small amount of squamous differentiation); peripheral palisading of nuclei at the margins of cell nests; retraction artefact (e.g., clefts) around cell nests; variable inflammatory infiltrate and ulceration; hyperkeratosis and/or ulceration; columns of parakeratosis optionally overlying atypical keratinocytes optionally separated by areas of orthokeratosis; basal atypical keratinocytes with varying degrees of overlying loss of maturation, hyperchromatism, pleomorphism, increased and abnormal mitoses, dyskeratosis-full thickness change may be called ‘bowenoid actinic keratosis’; variable superficial perivascular or lichenoid chronic inflammatory infiltrate; solar elastosis; hyperkeratosis, parakeratosis; acanthosis; full thickness epidermal involvement by atypical keratinocytes, with pale vacuolated or multinucleated cells; in some lesions, pagetoid spread at the margins; proliferation of atypical keratinocytes; invasion of dermis; variable degrees of keratinisation, optionally squamous eddies or keratin pearls; cyst lined by squamous epithelium, optionally flattened, with a granular layer; lamellated keratin within cyst; hyperpigmented elongated rete ridges; increased melanocytes; squamous lining but no granular layer; dense keratin content; frequent calcification; variable epidermal changes (e.g., atrophy, hyperplasia, papillomatosis, horn cysts); nests of melanocytes/naevus cells at the dermo-epidermal junction (e.g., junctional naevus) and/or in the dermis (e.g., compound naevus, dermal naevus); naevus cells in the epidermis confined to the basal layer, optionally at the tips of the rete ridges; generally round naevus cells that show decreasing size of both the cells and the cell nests with increasing depth in the dermis (e.g., maturation); inflammation, inflammation that varies based on trauma state; asymmetrical proliferation of melanocytes; atypical melanocytes invading upwards through epidermis and downwards into dermis; variable cytological atypia (e.g., loss of maturation, pleomorphism, hyperchromatism, increased mitoses, prominent nucleoli); epidermal hyperplasia (optionally mimicking basal cell carcinoma); hyperpigmented basal layer; circumscribed but poorly demarcated proliferation of spindled fibroblasts; histiocytes and few giant cells; variable amounts of collagen; focal epidermal hyperplasia with hyperkeratosis, parakeratosis and papillomatosis (not verruca plana); trichilemmal keratinization; koilocytes (e.g., keratinocytes in upper squamous layer with vacuoles, large cytoplasmic eosinophilic aggregates, and/or pyknotic nuclei); tangential sections showing squamous cells surrounded by inflamed stroma; older lesions lacking cytoplasmic changes; viral nuclear inclusions, basophilic viral nuclear inclusions; striking papillomatosis (e.g., upward displacement of dermal papillae), stratum corneum exhibits parakeratosis with pointed mounds resembling church spires, extravasated erythrocytes or hemosiderin; granular layer is thickened with prominent keratohyalin granules and keratinocytes displaying perinuclear clearing (e.g., koilocytosis); lymphocytic infiltrate in upper dermis; involuting lesions having chronic inflammatory infiltrates in dermis and epidermis with degenerative epithelial changes; invaginated with numerous coarse, basophilic, intracytoplasmic keratohyalin granules resembling molluscum bodies, or the like, or any combination thereof.

The term “disease,” as used herein, generally refers to an abnormal condition, or a disorder of a biological function or a biological structure such as an organ, that affects part or all of a subject. A disease may be caused by factors originally from an external source, such as infectious disease, or it may be caused by internal dysfunctions, such as autoimmune diseases. A disease can refer to any condition that causes pain, dysfunction, distress, social problems, and/or death to the subject afflicted. A disease may be an acute condition or a chronic condition. A disease may refer to an infectious disease, which may result from the presence of pathogenic microbial agents, including viruses, bacteria, fungi, protozoa, multicellular organisms, and aberrant proteins as prions. A disease may refer to a non-infectious disease, including but not limited to cancer and genetic diseases. In some cases, a disease can be cured. In some cases, a disease cannot be cured. In some cases, the disease is epithelial cancer. An epithelial cancer is a skin cancer including, but not limited to, non-melanoma skin cancers, such as basal cell carcinoma (BCC) and squamous cell carcinoma (SCC), and melanoma skin cancers.

The terms “epithelial tissue” and “epithelium,” as used herein, generally refer to the tissues that line the cavities and surface of blood vessels and organs throughout the body. Epithelial tissue comprises epithelial cells of which there are generally three shapes: squamous, columnar, and cuboidal. Epithelial cells can be arranged in a single layer of cells as simple epithelium comprising either squamous, columnar, or cuboidal cells, or in layers of two or more cells deep as stratified (layered), comprising either squamous, columnar, and/or cuboidal.

The term “cancer,” as used herein, generally refers to a proliferative disorder caused or characterized by a proliferation of cells which may have lost susceptibility to normal growth control. Cancers of the same tissue type usually originate in the same tissue and may be divided into different subtypes based on their biological characteristics. Non-limiting examples of categories of cancer are carcinoma (epithelial cell derived), sarcoma (connective tissue or mesodermal derived), leukemia (blood-forming tissue derived) and lymphoma (lymph tissue derived). Cancer may involve any organ or tissue of the body. Examples of cancer include melanoma, leukemia, astrocytoma, glioblastoma, retinoblastoma, lymphoma, glioma, Hodgkin's lymphoma, and chronic lymphocytic leukemia. Examples of organs and tissues that may be affected by various cancers include the pancreas, breast, thyroid, ovary, uterus, testis, prostate, pituitary gland, adrenal gland, kidney, stomach, esophagus, rectum, small intestine, colon, liver, gall bladder, head and neck, tongue, mouth, eye and orbit, bone, joints, brain, nervous system, skin, blood, nasopharyngeal tissue, lung, larynx, urinary tract, cervix, vagina, exocrine glands, and endocrine glands. In some cases, a cancer can be multi-centric. In some cases, a cancer can be a cancer of unknown primary (CUP).

The term “lesion,” as used herein, generally refers to an area(s) of disease and/or suspected disease, wound, incision, or surgical margin. Wounds may include, but are not limited to, scrapes, abrasions, cuts, tears, breaks, punctures, gashes, slices, and/or any injury resulting in bleeding and/or skin trauma sufficient for foreign organisms to penetrate. Incisions may include those made by a medical professional, such as but not limited to, physicians, nurses, mid-wives, and/or nurse practitioners, and dental professionals during treatment such as a surgical procedure.

The term “light,” as used herein, generally refers to electromagnetic radiation. Light may be in a range of wavelengths from infrared (e.g., about 700 nm to about 1 mm) through the ultraviolet (e.g., about 10 nm to about 380 nm). Light may be visible light. Alternatively, light may be non-visible light. Light may include wavelengths of light in the visible and non-visible wavelengths of the electromagnetic spectrum.

The term “ambient light,” as used herein, generally refers to the light surrounding an environment or subject, such as the light at a location in which devices, methods and systems of the present disclosure are used, such as a point of care location (e.g., a subject's home or office, a medical examination room, or operating room).

The term “optical axis” as used herein, generally refers to a line along which there may be some degree of rotational symmetry in an optical system such as a camera lens or microscope. The optical axis may be a line passing through the center of curvature of a lens or spherical mirror and parallel to the axis of symmetry. The optical axis herein is may also be referred to as the Z axis. For a system of simple lenses and mirrors, the optical axis may pass through the center of curvature of each surface and coincide with the axis of rotational symmetry. The optical axis may be coincident with the system's mechanical axis, as in the case of off-axis optical systems. For an optical fiber, the optical axis (also called as fiber axis) may be along the center of the fiber core.

The term “position,” as used herein, generally refers to a location on a plane perpendicular to the optical axis as opposed to a “depth” which is parallel to the optical axis. For example, a position of a focal point can be a location of the focal point in the x-y plane. Whereas a “depth” position can be a location along a z axis (optical axis). A position of a focal point can be varied throughout the x-y plane. A focal point can also be varied simultaneously along the z axis. The position may be a position of a focal point.

The term “position” can also refer to the position of an optical device (or housing) which can include: the location in space of the probe; the locations with respect to anatomical features of a subject; and the orientation or angle of the probe and/or its optics or optical axis. Position can mean the location or orientation of the probe in, on or near, tissue or tissue boundaries of a subject. Position can also mean a location with respect to other characteristics or features identified in a subject's tissue or with respect other data collected or observed from a subject's tissue. Position of an optical device can also mean the location and/or orientation of the probe or its optics with respect to tags, markers, or guides.

The term “focal point” or “focal spot” as used herein generally refers to a point of light on an axis of a lens or mirror of an optical element to which parallel rays of light converge. The focal point or focal spot can be in a tissue sample to be imaged, from which a return signal is generated that can be processed to create depth profiles.

The term “focal plane” as used herein, generally refers a plane formed by focal points directed along a scan path. The focal plane can be where the focal point moves in an X and/or Y direction, along with a movement in a Z direction wherein the Z axis is generally an optical axis. A scan path may also be considered a focal path that comprises at least two focal points that define a path that is non-parallel to the optical axis. For example, a focal path may comprise a plurality of focal points shaped as a spiral. A focal path as used herein may or may not be a plane and may be a plane when projected on an X-Z or Y-Z plane. The focal plane may be a slanted plane. The slanted plane may be a plane that is oriented at an angle with respect to an optical axis of an optical element (e.g., a lens or a mirror). The angle may be between about 0° and about 90°. The slanted plane may be a plane that has non-zero Z axis components.

The term “depth profile,” as used herein, generally refers to information or optical data derived from the generated signals that result from scanning a tissue sample. The scanning a tissue sample can be with imaging focal points extending in a parallel direction to an optical axis or z axis, and with varying positions on an x-y axis. The tissue sample can be, for example, in vivo skin tissue where the depth profile can extend across layers of the skin such as the dermis, epidermis, and subcutaneous layers. A depth profile of a tissue sample can include data that when projected on an X-Z or Y-Z plane creates a vertical planar profile that can translate into a projected vertical cross section image. The vertical cross section image of the tissue sample derived from the depth profile can be vertical or approximately vertical. In some cases, a depth profile provides varied vertical focal point coordinates while the horizontal focal point coordinates may or may not vary. A depth profile may be in the form of at least one plane at an angle to an optical plane (on an optical axis). For example, a depth profile may be parallel to an optical plane or may be at an angle less 90 degrees and greater than 0 degrees with respect to an optical plane. A depth profile may be generated using an optical device that is contacting a tissue at an angle. For example, the optical device may include a probe that contacts a tissue. The probe may include one or more objectives. The one or more objectives may contact a tissue. For example, a depth profile may not be perpendicular to the optical axis, but rather offset by the same degree as the angle the optical device is contacting the tissue. A depth profile can provide information at various depths of the sample, for example at various depths of a skin tissue. A depth profile can be provided in real-time. A depth profile may or may not correspond to a planar slice of tissue. A depth profile may correspond to a slice of tissue on a slanted plane. A depth profile may correspond to a tissue region that is not precisely a planar slice (e.g., the slice may have components in all three dimensions). A depth profile can be a virtual slice of tissue or a virtual cross section. A depth profile can be optical data scanned from in-vivo tissue. The data used to create a projected cross section image may be derived from a plurality of focal points distributed along a general shape or pattern. The plurality of distributed points can be in the form of a scanned slanted plane, a plurality of scanned slanted planes, or non-plane scan patterns or shapes (e.g., a spiral pattern, a wave pattern, or other predetermined or random or pseudorandom patterns of focal points.) The location of the focal points used to create a depth profile may be changed or changeable to track an object or region of interest within the tissue, that is detected or identified during scanning or related data processing. A depth profile may be formed from one or more distinct return signals or signals that correspond to anatomical features or characteristics from which distinct layers of a depth profile can be created. The generated signals used to form a depth profile can be generated from an excitation light beam. The generated signals used to form a depth profile can be synchronized in time and location. A depth profile may comprise a plurality of depth profiles where each depth profile corresponds to a particular signal or subset of signals that correspond to anatomical feature(s) or characteristics. The depth profiles can form a composite depth profile generated using signals synchronized in time and location. Depth profiles herein can be in vivo depth profiles wherein the optical data is obtained of in vivo tissue. A depth profile can be a composite of a plurality of depth profiles or layers of optical data generated from different generated signals that are synchronized in time and location. A depth profile can be a depth profile generated from a subset of generated signals that are synchronized in time and location with other subsets of generated signals. A depth profile can include one or more layers of optical data, where each of the layer corresponds to a different subset of signals. A depth profile or depth profile optical data can also include data from processing the depth profile, the optical device, optical device position, other sensors, or information identified and corresponding to the time of the depth profile or other pertinent information. Additionally, other data corresponding to subject information such as, for example, medical data, physical conditions, or other data or characteristics, can also be included with optical data of a depth profile. Depth profiles can be annotated depth profiles with annotations or markings.

The term “medical data” as used herein generally refers to the medical data of the subject comprising at least one medical data selected from the group consisting of a physical condition, medical history, test results, current and past occupations, age, sex, race, skin type, Fitzpatrick skin type, other metrics for skin health and appearance, nationality of the subject, environmental exposure, mental health, and medications. The physical conditions of the subject may be obtained through one or more medical instruments. The one or more medical instruments may include, but not limited to, stethoscopes, suction devices, thermometers, tongue depressors, transfusion kits, tuning forks, ventilators, watches, stopwatches, weighing scales, crocodile forceps, bedpans, cannulas, cardioverters, defibrillators, catheters, dialyzers, electrocardiogra machines, enema equipment, endoscopes, gas cylinders, gauze sponges, hypodermic needles, syringes, infection control equipment, instrument sterilizers, kidney dishes, measuring tapes, medical halogen penlights, nasogastric tubes, nebulizers, ophthalmoscopes, otoscopes, oxygen masks and tubes, pipettes, droppers, proctoscopes, reflex hammers, sphygmomanometers, spectrometers, dermatoscopes, and cameras. In some embodiments, the physical condition comprises vital signs of the subject. The vital signs may be measurements of the patient's basic body functions. The vital signs may include body temperature, pulse rate, respiration rate, and blood pressure. In some embodiments, the methods described herein further comprises receiving or using medical data of the subject.

The term “projected cross section image” as used herein generally refers to an image constructed from depth profile information projected onto the XZ or YZ plane to create an image plane. In this situation, there may be no distortion in depths of structures relative to the surface of the tissue. The projected cross section image may be defined by the portion of the tissue that is scanned. A projected cross section image can extend in a perpendicular direction relative to the surface of the skin tissue. The data used to create a projected cross section image may be derived from a scanned slanted plane or planes, and/or non-plane scan patterns, shapes (e.g., a spiral, a wave, etc.), or predetermined or random patterns of focal points.

The term “fluorescence,” as used herein, generally refers to radiation that can be emitted as the result of the absorption of incident electromagnetic radiation of one or more wavelengths (e.g., a single wavelength or two different wavelengths). In some cases, fluorescence may result from emissions from exogenously provided tags or markers. In some cases, fluorescence may result as an inherent response of one or more endogenous molecules to excitation with electromagnetic radiation.

The term “autofluorescence,” as used herein, generally refers to fluorescence from one or more endogenous molecules due to excitation with electromagnetic radiation.

The term “multi-photon excitation,” as used herein, generally refers to excitation of a fluorophore by more than one photon, resulting in the emission of a fluorescence photon. In some cases, the emitted photon is at a higher energy than the excitatory photons. In some cases, a plurality of multi-photon excitations may be generated within a tissue. The plurality of multi-photon excitations may generate a plurality of multi-photon signals. For example, cell nuclei can undergo a two-photon excitation. As another example, cell walls can undergo a three-photon excitation. At least a subset of the plurality of signals may be different. The different signals may have different wavelengths which may be used for methods described herein. For example, the different signals (e.g., two-photon or three-photon signals) can be used to form a map which may be indicative of different elements of a tissue. In some cases, the map is used to train machine learning based diagnosis algorithms.

The terms “second harmonic generation” and “SHG,” as used herein, generally refer to a nonlinear optical process in which photons interacting with a nonlinear material are effectively “combined” to form new photons with about twice the energy, and therefore about twice the frequency and about half (½) the wavelength of the initial photons.

The terms “third harmonic generation” and “THG,” as used herein, generally refer to a nonlinear optical process in which photons interacting with a nonlinear material are effectively “combined” to form new photons with about three times the energy, and therefore about three times the frequency and about a third (⅓) the wavelength of the initial photons.

The term “reflectance confocal microscopy” or “RCM,” as used herein, generally refers to a process of collecting and/or processing reflected light from a sample (e.g., a tissue or any components thereof). The process may be a non-invasive process where a light beam is directed to a sample and returned light from the focal point within the sample (“RCM signal”) may be collected and/or analyzed. The process may be in vivo or ex vivo. RCM signals may trace a reverse direction of a light beam that generated them. RCM signals may be polarized or unpolarized. RCM signals may be combined with a pinhole, single mode fiber, multimode fiber, intersecting excitation and collection optical pathways, or other confocal arrangements that restrict the light collected to that portion arising from the focal point. A confocal microscopy arrangement as defined herein, can be an arrangement that focuses the restricted focal point arising light at a finite distance or at an effectively infinite distance through an aperture and/or optical fiber or fiber bundle for collection. In some embodiments, an effectively infinite distance the light from the focus can be collimated and pass through an aperture/fiber approximately equal or similar in diameter to the collimated beam. While RCM generally refers to collecting reflected light, it can also refer to a confocal arrangement that can collect transmitted or fluoresced light.

The term “polarized light,” as used herein, generally refers to light with waves oscillating in one plane. Unpolarized light can generally refer to light with waves oscillating in more than one plane.

The term “excitation light beam,” as used herein, generally refers to the focused light beam directed to tissue to create a generated signal. An excitation light beam can be a single beam of light. An excitation light beam can be a pulsed single beam of light. An excitation beam of light can be a plurality of light beams. The plurality of light beams can be synchronized in time and location as described herein. An excitation beam of light can be a pulsed beam or a continuous beam or a combination one or more pulsed and/or continuous beams that are delivered simultaneously to a focal point of tissue to be imaged. The excitation light beam can be selected depending upon the predetermined type of return signal or generated signal as described herein. An excitation beam of light as used herein includes an illumination light used to generate a reflected or transmitted signal, e.g., a confocal microscopy signal.

The term “generated signal” as used herein generally refers to a signal that is returned from the tissue resulting from direction of focused light (e.g., excitation light) to the tissue and including but not limited to reflected, absorbed, scattered, or refracted light. Generated signals may include, but are not limited to, endogenous signals arising from the tissue itself or signals from exogenously provided tags or markers. Generated signals may arise in either in vivo or ex vivo tissue. Generated signals may be characterized as either single-photon generated signals or multi-photon generated signals as determined by the number of excitation photons that contribute to a signal generation event. Single-photon generated signals may include but are not limited to reflectance confocal microscopy (“RCM”) signals, single-photon fluorescence, and single-photon autofluorescence. Single-photon generated signals, such as RCM, can arise from either a continuous light source, or a pulsed light source, or a combination of light sources that can be either pulsed or continuous. Single-photon generated signals may overlap. Single-photon generated signals may be deconvoluted. Multi-photon generated signals may be generated by at least 2, 3, 4, 5, or more photons. Multi-photon generated signals may include but are not limited to second harmonic generation, two-photon autofluorescence, two-photon fluorescence, third harmonic generation, three-photon autofluorescence, three-photon fluorescence, multi-photon autofluorescence, multi-photon fluorescence, and coherent anti-stokes Raman spectroscopy. Multi-photon generated signals can arise from either a single pulsed light source, or a combination of pulsed light sources as in the case of coherent anti-stokes Raman spectroscopy. Multi-photon generated signals may overlap. Multi-photon generated signals may be deconvoluted. Other generated signals may include but are not limited to Optical Coherence Tomography (OCT), single or multi-photon fluorescence/autofluorescence lifetime imaging, polarized light microscopy signals, additional confocal microscopy signals, and ultrasonography signals. Single-photon and multi-photon generated signals can be combined with polarized light microscopy by selectively detecting the components of the generated signals that are either linearly polarized light, circularly polarized light, unpolarized light, or any combination thereof. Polarized light microscopy may further comprise blocking all or a portion of the generated signal possessing a polarization direction parallel or perpendicular to the polarization direction of the light used to generate the signals or any intermediate polarization direction. Generated signals as described herein may be combined with confocal techniques utilizing a pinhole, single mode fiber, multimode fiber, intersecting excitation and collection optical pathways, or other confocal arrangements that restrict the light detected from the generated signal to that portion of the generated signal arising from the focal point. For example, a pinhole can be placed in a Raman spectroscopy instrument to generate confocal Raman signals. Raman spectroscopy signals may generate different signals based at least in part on different vibrational states present within a sample or tissue. Optical coherence tomography signals may use light comprising a plurality of phases to image a tissue. Optical coherence tomography may be likened to optical ultrasonography. Ultrasonography may generate a signal based at least in part on the reflection of sonic waves from features within a sample (e.g., a tissue).

The term “contrast enhancing agent,” as used herein, generally refers to any agent such as but not limited to fluorophores, metal nanoparticles, nanoshell composites and semiconductor nanocrystals that can be applied to a sample to enhance the contrast of images of the sample obtained using optical imaging techniques. Fluorophores can be antibody targeted fluorophores, peptide targeted fluorophores, and fluorescent probes of metabolic activity. Metallic nanoparticles can comprise metals such as gold and silver that can scatter light. Nanoshell composites can include nanoparticles comprising a dielectric core and metallic shell. Semiconductor nanocrystals can include quantum dots, for example quantum dots containing cadmium selenide or cadmium sulfide. Other contrasting agents can be used herein as well, for example by applying acetic acid to tissue.

The term “in real-time” and “real-time,” as used herein, generally refers to immediate, rapid, not requiring operator intervention, automatic, and/or programmed. Real-time may include, but is not limited to, measurements in femtoseconds, picoseconds, nanoseconds, milliseconds, seconds, as well as longer, and optionally shorter, time intervals.

The term “tissue” as used herein, generally refers to any tissue or content of tissue. A tissue may be a sample that is healthy, benign, or otherwise free of a disease. A tissue may be a sample removed from a subject, such as a tissue biopsy, a tissue resection, an aspirate (such as a fine needle aspirate), a tissue washing, a cytology specimen, a bodily fluid, or any combination thereof. The tissue from which images can be obtained can be any tissue or content of tissue of the subject including but not limited to connective tissue, epithelial tissue, organ tissue, muscle tissue, ligaments, tendons, a skin tissue, breast tissue, bladder, kidney tissue, liver tissue, colon tissue, thyroid tissue, cervical tissue, prostate tissue, lung tissue, cardiac tissue, heart tissue, muscle tissue, pancreas tissue, anal tissue, bile duct tissue, a bone tissue, bone marrow, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, neurons, cells representative of a blood-brain barrier, blood, hair, nails, keratin, collagen, or any combination thereof.

The term “imaging subject” as used herein, generally refers to an object, material, specimen, sample or tissue, including but not limited to in vivo or ex vivo tissue.

The term “reflector” as used herein, generally refers to an element that can be configured to reflect at least a portion of a beam of light, including by not limited to an individual structural component, unit or element. A reflector may be a material covering a surface where the material is configured to reflect at least a portion of a beam of light. A reflector can be a retroreflector, mirror or other optical element configured to reflect at least a portion of a beam of light.

The term “collector” as used herein, generally refers to an element that can be configured to collect at least a portion of a beam of light or other signal. A collector can refer to a sensor including but not limited to an optical sensor that can collect a portion of an optical signal.

The term “numerical aperture” as used herein, generally refers to a dimensionless number that characterizes the range of angles over which the system can accept or emit light. Numerical aperture may be used in microscopy to describe the acceptance cone of an objective (and hence its light-gathering ability and resolution).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Systems and Devices for Light Filtering and Imaging

In an aspect, the present disclosure provides a device for light filtering and/or imaging. The device may comprise a probe. The probe may be configured to direct a beam of light from a light source to an imaging subject. The probe may be configured to direct the beam of light such that the beam of light contacts the imaging subject. The probe may be configured to collect light from the imaging subject upon the beam of light contacting the imaging subject. The device may further comprise a light filtering device. The light filtering device may be in optical communication (e.g., in an optical pathway) with the probe. The light filtering device may comprise a chamber and a plurality of reflectors. The chamber may comprise an input and an output. The input may be configured to receive the light collected by the probe. The output may be configured to transmit the light away from the chamber. The plurality of reflectors may be disposed in the chamber. The plurality of reflectors may be configured to direct the light received. For example, the plurality of reflectors may be configured to direct the light (received from the probe) in an optical path from the input of the chamber to the output of the chamber. The light may be directed via reflectance between reflectors of the plurality of reflects. The chamber may be configured to reject out of focus light along the optical path between the input and the output via reflectance of the light between the plurality of reflectors.

In an aspect, the present disclosure provides devices for light filtering and imaging. In an aspect, the present disclosure provides devices for light filtering for confocal imaging. The light filtering device may include a chamber having a plurality of reflectors. The chamber may be configured to receive or may receive a beam of light. The chamber may be configured to filter out unfocused light from the beam of light. Alternatively, or in addition, the chamber may comprise a light filtering device. Each of the reflectors of the plurality of reflectors may be disposed in a location in the chamber. Each reflector may be in a unique location from all other reflectors, or one or more reflectors may be located in substantially the same location. A beam of light directed into the reflection chamber may follow a path defined by path segments between the reflectors whereby the beam of light travels from one of the reflectors to another to define an optical path from the input into the chamber to the output from the chamber. The light filtering device, or the chamber, may be configured to reject out of focus light along the optical path. A beam of light directed into the reflection chamber may follow a path defined by path segments between the units whereby the beam of light travels from one of the reflectors to another in the path that returns to the first reflector at least one time before reaching the output of the chamber. The path may comprise two or more path segments, where a path segment is a portion of the optical path, the portion being between two reflectors of the plurality of reflectors, between the input of the chamber and a reflector, or between a reflector and the output of the chamber.

In another aspect, the present disclosure provides a device for light filtering and imaging. According to some embodiments, the device for light filtering and imaging may be a confocal imaging device. The imaging device may include a light source, a probe, a light filtering device, and a collector. The light source may be configured to provide or may provide a beam of light to an imaging subject. The probe may be in optical communication (e.g., in an optical pathway) with the light source. The probe may be configured to direct or may direct the beam of light from the light source to the imaging subject. The light filtering device may be in optical communication with the probe. The light filtering device may include a chamber with a plurality of reflectors. The light filtering device may be configured to receive or may receive the beam of light from the probe. The light filtering device may further be configured such that a pathlength that the beam of light traverses from the opening, between the reflectors and to an output. The collector may be in optical communication (e.g., in an optical pathway) with the light filtering device. The collector may be configured to collect or may collect the beam of light from the light filtering device. The collector may be coupled to or located at the output of the light filtering device. The collector may include or may be coupled to a sensor and/or an image processor. The beam of light may be usable to generate an image of the imaging subject.

In another aspect, the present disclosure provides systems for light filtering and imaging. The system may include a light source, a light filtering device, and one or more computer processors. The light source may be configured to generate a beam of light. The light filtering device may be in optical communication with the light source. The light filtering device may include a chamber, and a plurality of reflectors. The chamber may be configured to receive or may receive a beam of light from the light source. The first reflector may be disposed in the chamber. The first reflector may be configured to direct or may direct at least a portion of the beam of light from the first reflector in the chamber to a second reflector in the chamber. The second reflector may be configured to direct or may direct another portion of the beam of light from the second location in the chamber to the first reflector in the chamber. The chamber comprising the plurality of reflectors (e.g., the first reflector and second reflector) may have a longest internal dimension. The longest internal dimension may be a length of the inside of the chamber from one point to a second point. The longest internal dimension may be longer than any other length between any two other points within the chamber. The beam of light may be directed in an optical path between the input of the chamber to the output of the chamber via reflectance between one or more reflectors of the plurality of reflectors within the chamber. The length of the optical path may be the sum of the length of the segments of the optical path. In some cases, the length of the optical path may be greater than the longest internal dimension of the chamber. The length of the optical path may be greater than or equal to about 0.01 meters, 0.05 meters, 0.08 meters, 0.1 meters, 0.14 meters, 0.17 meters, 0.20 meters, 0.25 meters, 0.30 meters, 0.35 meters, 0.40 meters, 0.50 meters, 0.60 meters, 0.75 meters, 0.90 meters, 1 meter, 1.25 meters, 1.5 meters, 1.75 meters, 2 meters, 2.5 meters, 3 meters, 5 meters, 10 meters, or greater. The length of the optical path may be less than or equal to about 15 meters, 11 meters, 6 meters, 4 meters, 2 meters, 1 meter, 0.80 meters, 0.65 meters, 0.55 meters, 0.45 meters, 0.5 meters, 0.25 meters, 0.20 meters, 0.18 meters, 0.16 meters, 0.13 meters, 0.10 meters, 0.07 meters, 0.04 meters, 0.02 meters, or less. The length of the optical path may be between any two lengths described above, for example between about 0.25 meters and 1 meter.

In some cases, the second reflector may direct the light beam back to the first reflector at least once before the beam of light is directed out of the chamber. The one or more computer processors may be operatively coupled to the light source and the light filtering device. The one or more computer processors may be individually or collectively programmed to process the beam of light to generate an image.

Devices described herein can be used in any type of microscopy, including where confocal imaging is used. Devices described herein can be used in any laser scanning microscope, with or without needle tip probes or other probes, and/or with or without a pulsed laser. Devices described herein can also be used in any type of tissue imaging, (e.g., as tissue is defined herein). Moreover, a reflector of a device described herein can be a reflective surface or material. In addition, the reflector can be an individual structural component or sub-unit, and/or can be a material covering an inside surface of a chamber.

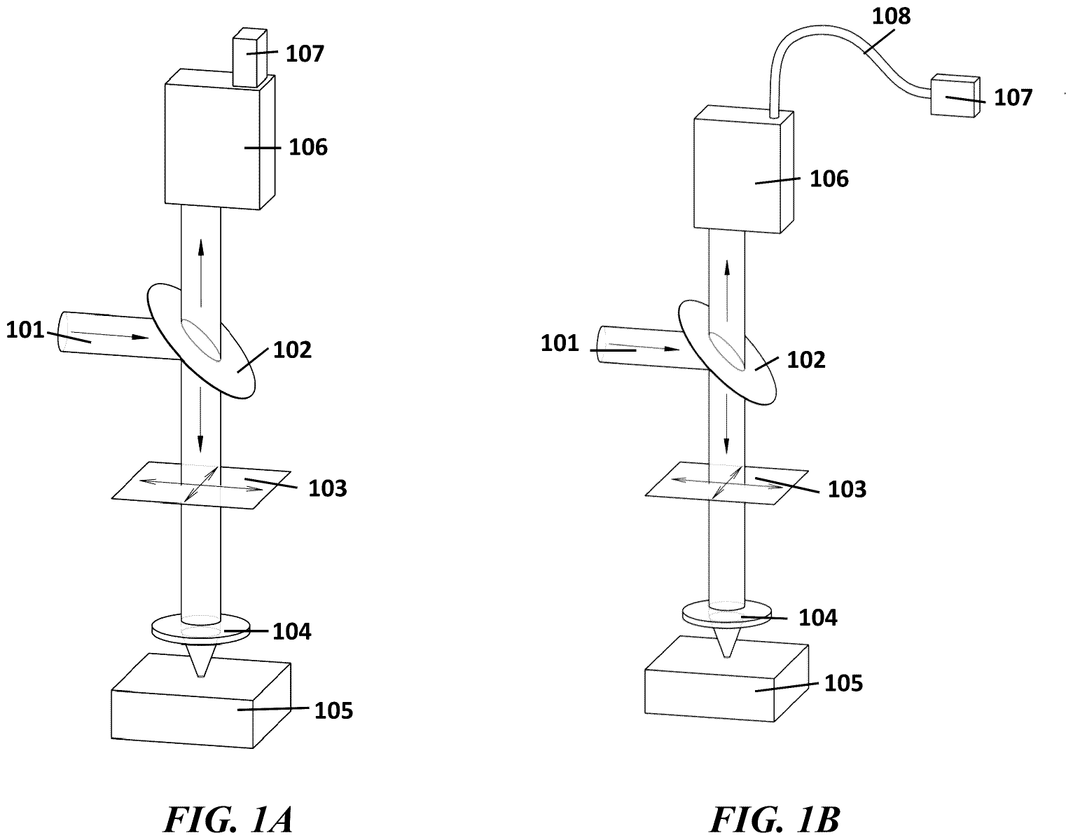

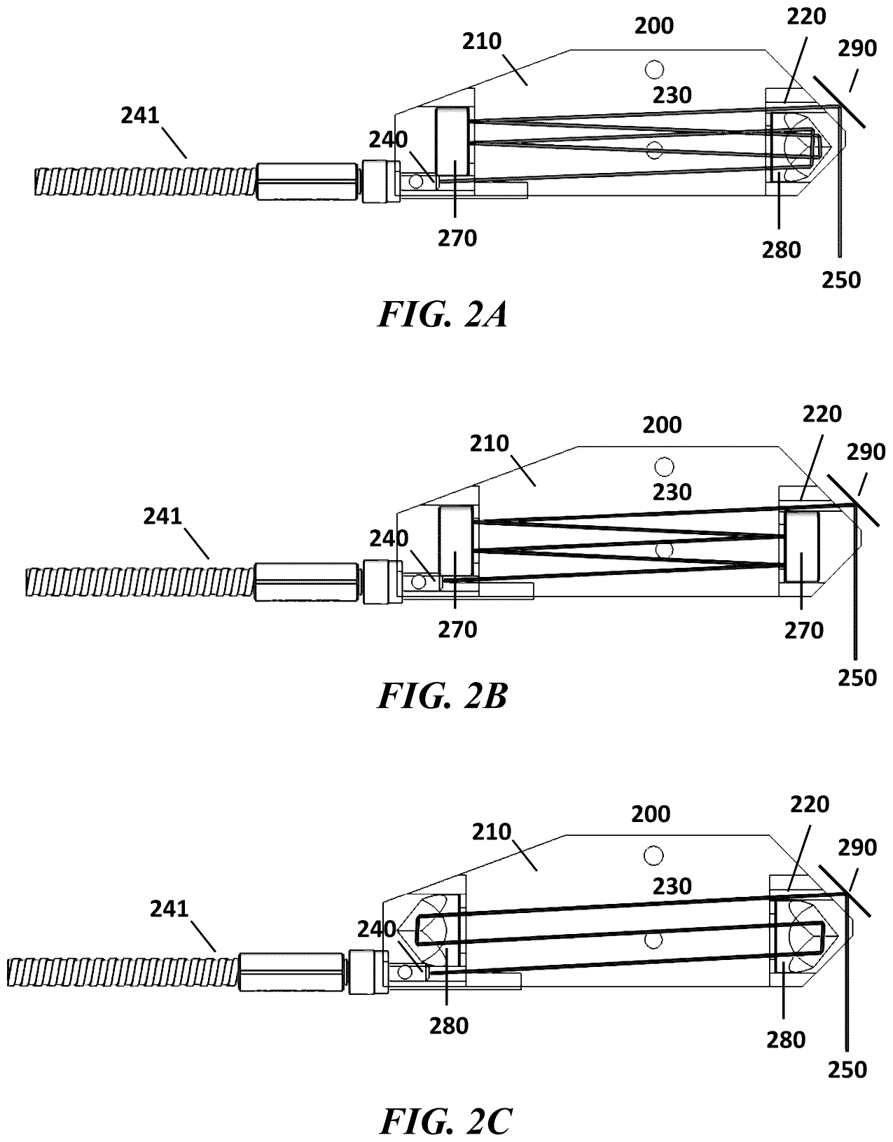

Example devices for confocal optical imaging are shown in FIGS. 1A and 1B. The optical device may include a light source 101 that provides a beam of light to a beam splitter 102. The beam splitter 102 may direct a portion of the beam of light to a scanning unit 103. The scanning unit 103 may scan the beam of light in two dimensions (e.g., x- and y-direction scanning) across an imaging subject 105. The scanning unit 103 may provide depth scanning (e.g., z-direction scanning) of the imaging subject 105. The scanning unit 103 may scan in any combination of x, y, and z directions. The scanning unit may be configured to scan three-dimensional images, two-dimensional images, slices of an image in a direction (e.g., two-dimensional slices in the x-y plane repeated in the z direction) or other kind of imaging that uses three-dimensional or two-dimensional scanning. The scanning unit 103 may provide multiple layers of scanning to form a composite image. The scanning unit 103 may provide a multidimensional image, e.g., a 2 or 3 dimensional image and/or time dependent image. The scanning unit 103 may provide the beam of light to a probe 104 prior to the probe 104 providing the beam of light to the imaging subject 105. The probe 104 may include an objective. Contacting the imaging subject 105 with the beam of light may generate a light signal. The generated light signal may be indicative or one or more properties of the imaging subject. The light signal may be usable for imaging the imaging subject 105. The probe 104 may operate in part as a collector that collects light from a subject or specimen. The generated light signal may be collected from the imaging subject 105 by the probe 104. The probe 104 may direct the generated light signal to the beam splitter 102. The beam splitter 102 may provide at least a portion of the light signal to a light filtering device 106. The light filtering device 106 may generate a pathlength or an effective pathlength of the light signal that is larger than a dimension of the light filtering device 106. According to some embodiments, light filtering device 106 may include one or more reflectors (e.g., mirrors, retroreflectors or coated surfaces) that permit the light signal (e.g., beam of light) to reflect between the reflectors to form an optical path having a path length. Repeatedly reflecting the signal between the reflectors may increase the pathlength of the light signal multiple times to filter out unfocused or uncollimated light. As shown in FIG. 1A, the light filtering device 106 may comprise a chamber as described in greater detail with reference to FIGS. 2A and 2B. The light filtering device 106 may generate an optical path, pathlength or an effective pathlength of the light signal that is larger than a largest linear dimension of the inside of a chamber. As shown in FIG. 1A, the light filtering device 106 may be coupled to or integrated with a collector 107. The collector 107 may collect light from the light filtering device 106 at the end of the optical path. Alternatively, or in addition, and as shown in FIG. 1B, the light filtering device may be coupled to a fiber (e.g., an optic fiber bundle) 108 included as part of or coupled to the collector 107.

Examples of optical devices or imaging devices described with respect to FIG. 1A and FIG. 1B include those that can be used in any type of microscopy, including where imaging is used. Devices described in FIG. 1A and FIG. 1B herein can be used in any laser scanning microscope, with or without needle tip probes or other probes, and/or with or without a pulsed laser. Devices described in FIG. 1A and FIG. 1B herein can also be used in any type of tissue imaging. Devices described in FIG. 1A and FIG. 1B herein can be used in confocal imaging. Examples of optical devices or imaging devices described with respect to FIG. 2A to FIG. 15 herein.

According to embodiments of the present invention, a light filtering device, including a chamber (e.g., a reflectance chamber), is provided to remove out of focus light from a collected reflected, transmitted, fluoresced or autofluoresced light signal. In some embodiments, the collected light signal is a confocal microscopy signal. In some embodiments, the collected light signal is generated from a single photon excitation light signal focused on an imaging subject (e.g., tissue) to be imaged. In some embodiments, the reflection chamber is configured to be used with an optical imaging device. In some embodiments, the optical imaging device is a handheld optical device. In some embodiments, the optical device is portable. In some embodiments the optical device is a handheld device. As the collected light passes through the chamber (e.g., reflection chamber) and reflects between reflectors, out of focus light is dispersed. The in-focus light may then pass through an output of the chamber into a collector. The output of the chamber may include an output aperture of the chamber. The output aperture may be an opening at the end of a reflection path. The output aperture may be an interface with or an input of a collection optic, fiber (e.g., array of collection fibers or an optic fiber bundle) at an end of a reflection path, The output aperture may be an interface with or an input of a sensor, at an end of a reflection path. In some embodiments, the output aperture or the collection fiber can have a smallest diameter that is approximately equal to the diameter of the collimated beam of in focus light. In some embodiments, the cross sectional diameter or dimension of a smallest output aperture or fiber can be approximately 10 mm, 9 mm, 8 mm, 7 mm, 6 mm, 5 mm, 4 mm, 3 mm, 2 mm, 1.5 mm, 1 mm, 0.5 mm, or 0.1 mm in diameter. In some embodiments, the smallest cross section dimension of the smallest output aperture or fiber can be approximately 10 mm, 9 mm, 8 mm, 7 mm, 6 mm, 5 mm, 4 mm, 3 mm, 2 mm, 1.5 mm, 1 mm, 0.5 mm, or 0.1 mm in diameter. In some embodiments, the cross section of the output (and output aperture or input collection fiber) may be a slit or have a slit shape. According to some embodiments, the collected light may not be focused following collection into the imaging device. According to some embodiments, the collected light may not be further focused within the chamber to the level of the selected resolution. For example, for a resolution of about 1 to 10 microns, a collected light beam and/or collection fiber or fiber bundle may have a cross-sectional dimension (e.g., diameter) of 1.5 millimeters (mm) and/or a smallest cross sectional diameter of about 0.2 mm. In some embodiments, the output aperture or the collection fiber may have a cross-sectional dimension (e.g., diameter) similar in scale to the collimated beam of in-focus light. In some embodiments, the output aperture or fiber may have a smallest cross-sectional dimension (e.g., diameter) of less than or equal to about 10 mm, 9 mm, 8 mm, 7 mm, 6 mm, 5 mm, 4 mm, 3 mm, 2 mm, 1.5 mm, 1 mm, 0.5 mm, 0.1 mm, or less. The output aperture or fiber may have a smallest cross-sectional dimension (e.g., diameter) of greater than or equal to about 0.1 mm, 0.5 mm, 1 mm, 1.5 mm, 2 mm, 3 mm, 4 mm, 5 mm, 6 mm, 7 mm, 8 mm, 9 mm, 10 mm, or more. The output aperture or fiber may have a smallest cross-sectional dimension (e.g., diameter) of from about 0.1 mm to about 0.5 mm, 0.1 mm to 1 mm, 0.1 mm to 1.5 mm, 0.1 mm to 2 mm, 0.1 mm to 3 mm, 0.1 mm to 4 mm, 0.1 mm to 5 mm, 0.1 mm to 6 mm, 0.1 mm to 7 mm, 0.1 mm to 8 mm, 0.1 mm to 9 mm, 0.1 mm to 10 mm, 0.5 mm to 1 mm, 0.5 mm to 1.5 mm, 0.5 mm to 2 mm, 0.5 mm to 3 mm, 0.5 mm to 4 mm, 0.5 mm to 5 mm, 0.5 mm to 6 mm, 0.5 mm to 7 mm, 0.5 mm to 8 mm, 0.5 mm to 9 mm, 0.5 mm to 10 mm, 1 mm to 1.5 mm, 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 1.5 mm to 2 mm, 1.5 mm to 3 mm, 1.5 mm to 4 mm, 1.5 mm to 5 mm, 1.5 mm to 6 mm, 1.5 mm to 7 mm, 1.5 mm to 8 mm, 1.5 mm to 9 mm, 1.5 mm to 10 mm, 2 mm to 3 mm, 2 mm to 4 mm, 2 mm to 5 mm, 2 mm to 6 mm, 2 mm to 7 mm, 2 mm to 8 mm, 2 mm to 9 mm, 2 mm to 10 mm, 3 mm to 4 mm, 3 mm to 5 mm, 3 mm to 6 mm, 3 mm to 7 mm, 3 mm to 8 mm, 3 mm to 9 mm, 3 mm to 10 mm, 4 mm to 5 mm, 4 mm to 6 mm, 4 mm to 7 mm, 4 mm to 8 mm, 4 mm to 9 mm, 4 mm to 10 mm, 5 mm to 6 mm, 5 mm to 7 mm, 5 mm to 8 mm, 5 mm to 9 mm, 5 mm to 10 mm, 6 mm to 7 mm, 6 mm to 8 mm, 6 mm to 9 mm, 6 mm to 10 mm, 7 mm to 8 mm, 7 mm to 9 mm, 7 mm to 10 mm, 8 mm to 9 mm, 8 mm to 10 mm, or 9 mm to 10 mm.

In some embodiments, the collimated light beam may have a cross-sectional dimension (e.g., diameter) of less than or equal to about 10 mm, 9 mm, 8 mm, 7 mm, 6 mm, 5 mm, 4 mm, 3 mm, 2 mm, 1.5 mm, 1 mm, 0.5 mm, 0.1 mm, or less. The collimated light beam may have a cross-sectional dimension (e.g., diameter) of greater than or equal to about 0.1 mm, 0.5 mm, 1 mm, 1.5 mm, 2 mm, 3 mm, 4 mm, 5 mm, 6 mm, 7 mm, 8 mm, 9 mm, 10 mm, or more. The fiber may have a cross-sectional dimension (e.g., diameter) of from about 0.1 mm to about 0.5 mm, 0.1 mm to 1 mm, 0.1 mm to 1.5 mm, 0.1 mm to 2 mm, 0.1 mm to 3 mm, 0.1 mm to 4 mm, 0.1 mm to 5 mm, 0.1 mm to 6 mm, 0.1 mm to 7 mm, 0.1 mm to 8 mm, 0.1 mm to 9 mm, 0.1 mm to 10 mm, 0.5 mm to 1 mm, 0.5 mm to 1.5 mm, 0.5 mm to 2 mm, 0.5 mm to 3 mm, 0.5 mm to 4 mm, 0.5 mm to 5 mm, 0.5 mm to 6 mm, 0.5 mm to 7 mm, 0.5 mm to 8 mm, 0.5 mm to 9 mm, 0.5 mm to 10 mm, 1 mm to 1.5 mm, 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 1.5 mm to 2 mm, 1.5 mm to 3 mm, 1.5 mm to 4 mm, 1.5 mm to 5 mm, 1.5 mm to 6 mm, 1.5 mm to 7 mm, 1.5 mm to 8 mm, 1.5 mm to 9 mm, 1.5 mm to 10 mm, 2 mm to 3 mm, 2 mm to 4 mm, 2 mm to 5 mm, 2 mm to 6 mm, 2 mm to 7 mm, 2 mm to 8 mm, 2 mm to 9 mm, 2 mm to 10 mm, 3 mm to 4 mm, 3 mm to 5 mm, 3 mm to 6 mm, 3 mm to 7 mm, 3 mm to 8 mm, 3 mm to 9 mm, 3 mm to 10 mm, 4 mm to 5 mm, 4 mm to 6 mm, 4 mm to 7 mm, 4 mm to 8 mm, 4 mm to 9 mm, 4 mm to 10 mm, 5 mm to 6 mm, 5 mm to 7 mm, 5 mm to 8 mm, 5 mm to 9 mm, 5 mm to 10 mm, 6 mm to 7 mm, 6 mm to 8 mm, 6 mm to 9 mm, 6 mm to 10 mm, 7 mm to 8 mm, 7 mm to 9 mm, 7 mm to 10 mm, 8 mm to 9 mm, 8 mm to 10 mm, or 9 mm to 10 mm.

In some embodiments the ratio of the smallest cross sectional diameter of the output aperture to the cross sectional diameter of the collimated beam of light collected at the output is greater than or equal to 1:20, 1:19, 1:18, 1:17, 1:16, 1:15 1:14, 1:13, 1:12, 1:11, 1:10, 1:9, 1:8, 1:7, 1:6, 1:5, 1:4, 1:4, 1:3, 1:2, or 1:1 In some embodiments the ratio of the smallest cross sectional diameter of the collection fiber to the cross sectional diameter of the collimated beam of light is greater than or equal to about 1:20, 1:19, 1:18, 1:17, 1:16, 1:15 1:14, 1:13, 1:12, 1:11, 1:10, 1:9, 1:8, 1:7, 1:6, 1:5, 1:4, 1:4, 1:3, 1:2, or 1:1. In some embodiments, the ratio of the smallest cross sectional diameter of the output aperture collection fiber to the cross sectional diameter of the collimated beam of light is between any two values described above, for example between about 1:3 and about 1:2.

FIG. 2A illustrates an example of a filtering device 200 configured to remove out of focus light from a collected light sample 250. The collected light sample 250 may include, for example, reflected light, transmitted light, fluorescent light, or any combination thereof resulting from an illumination light beam focused at a plane or location within an imaging subject (e.g., tissue sample, in vivo or ex vivo.) The filtering device 200 may comprise a chamber 210, an input 220 through which the collected light beam is directed into the chamber 210, and an output 240 configured to receive in-focus light passing along a path 230 through the chamber 210. The output 240 may include an output aperture. The output 240 may be, may include, or may be configured to direct the light to, a collector. Alternatively, or in addition the reflection chamber may include one or more waveguides that may permit light to enter the chamber or leave the chamber via an output. An input and/or output of the chamber may be coupled to a waveguide or may comprise a waveguide.

An alignment mirror 290 may be used to direct the light sample 250 through the input 220 into the reflection chamber 210. The alignment mirror 290 can move the direction of the light and thus control the angle of the light entering into the reflection chamber. The mirror 290 can alter the position of focus of the light beam on an x-y plane or simply on a single x- or y-axis. The alignment of the light entering into the chamber may not require any additional focus or may not require aligning the focus on the z-axis.

The chamber 210 may comprise a plurality of reflectors 270, 280. In an example, the chamber 210 may include at least 2, 3, 4, 5, 6, 7, 8, 9, 10, or more reflectors. The reflectors may be arranged in any configuration. The reflectors may have any arrangement within a chamber where light is reflected to form an optical path. For example, two or more reflectors may be configured in a parallel arrangement, circular configuration, or other shape with respect to other of the reflectors.

Examples of a reflector includes but are not limited to a mirror, a retroreflector, and a coated surface. A reflector may have a reflective surface of any shape. For example, the reflector may have a reflective surface with a circular, square, rectangular, elliptical, or any other shape. The reflective surface may have a dimension (e.g., width, height, diameter, etc.) of less than or equal to about 5 cm, 4 cm, 3 cm, 2 cm, 1.5 cm, 1 cm, 0.5 cm, or less. The reflective surface may have a dimension (e.g., width, height, diameter, etc.) from about 0.5 cm to 1 cm, 0.5 cm to 1.5 cm, 0.5 cm to 2 cm, 0.5 cm to 3 cm, 0.5 cm to 4 cm, 0.5 cm to 5 cm, 1 cm to 1.5 cm, 1 cm to 2 cm, 1 cm to 3 cm, 1 cm to 4 cm, 1 cm to 5 cm, 1.5 cm to 2 cm, 1.5 cm to 3 cm, 1.5 cm to 4 cm, 1.5 cm to 5 cm, 2 cm to 3 cm, 2 cm to 4 cm, 2 cm to 5 cm, 3 cm to 4 cm, 3 cm to 5 cm, or 3 cm to 5 cm. In an example, the reflective surface may have a dimension (e.g., width, height, diameter, etc.) of less than or equal to about 2 cm. In another example, the reflective surface may have a dimension (e.g., width, height, diameter, etc.) from bout 0.5 cm to 2 cm.

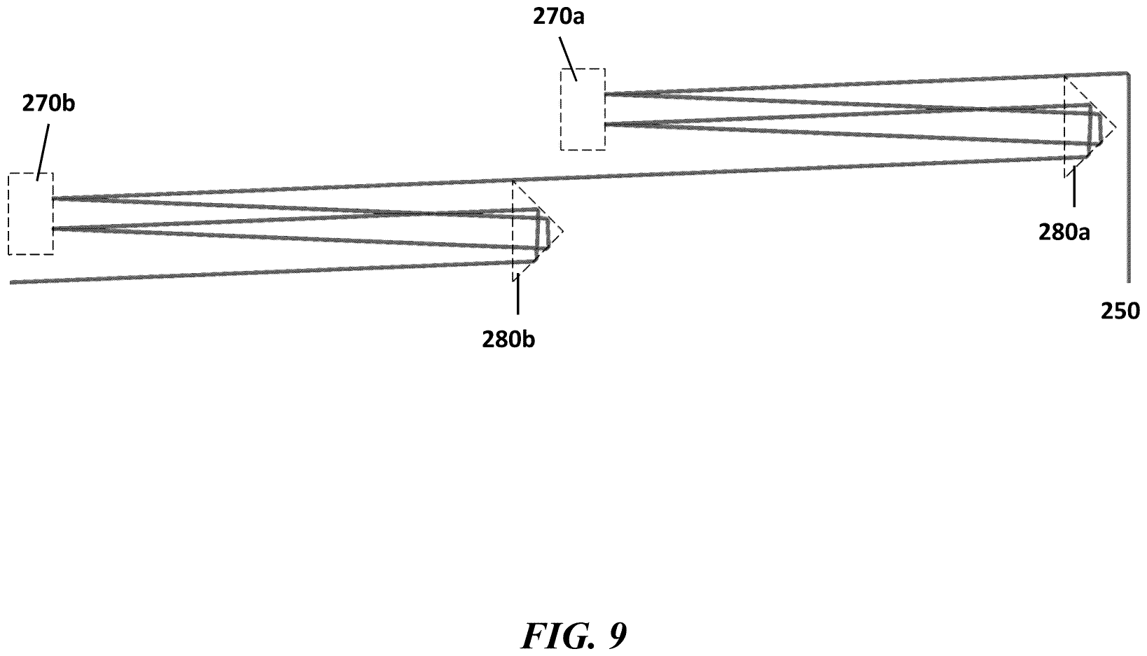

FIG. 2A illustrates an arrangement of opposing interfacing parallel reflectors 270, 280. However, as described above, more than two reflectors can be used in an arrangement of interfacing reflectors, or the reflector arrangements can be in series, in parallel or in non-parallel orientations, or combination of orientations. Some examples of multiple reflector or multiple chamber arrangements are illustrated in FIG. 9. As shown in FIGS. 2A-2C, the reflectors 270, 280 may include both mirrors 270 and retroreflectors 280 (see, e.g., FIG. 2A), mirrors 270 (see, e.g., FIG. 2B), or retroreflectors (see, e.g., FIG. 2C). Additionally or alternatively, reflective coated surfaces within a chamber may be used as reflectors. The collected light enters the reflection chamber 210 through the input 220 and the in-focus or collimated light is reflected a plurality of times between the at least two reflectors 270, 280. As the light moves between reflectors 270, 280 within the chamber, out of focus, non-collimated, diverging or converging light deviates from the collimated trajectory or disperses across an expanded cross section as it reaches the location of the output 240 of the chamber and input of an optical fiber 241. The optical fiber 241 collects primarily the in-focus collimated light. The output 240 may include an output aperture. The output 240 is illustrated in FIG. 2A-2C as the input to or interface with a collector 242. The collector 242 may include a sensor. An optical fiber 241 of a collector 242 may be positioned at the output 240 to collect collimated light at the end of the optical path where the output 240 of the chamber is the input of the fiber 241. However, an output aperture may be located in front of the optical fiber 241 with respect to the optical path 230 of the collimated beam. An output aperture may or may not have a larger or smaller opening dimension than an optical fiber 241 input or an input of the collector 242.

According to some embodiments, for example as shown in FIGS. 2A-2C, a first and second reflector may be disposed along an axis parallel to a length of the chamber.

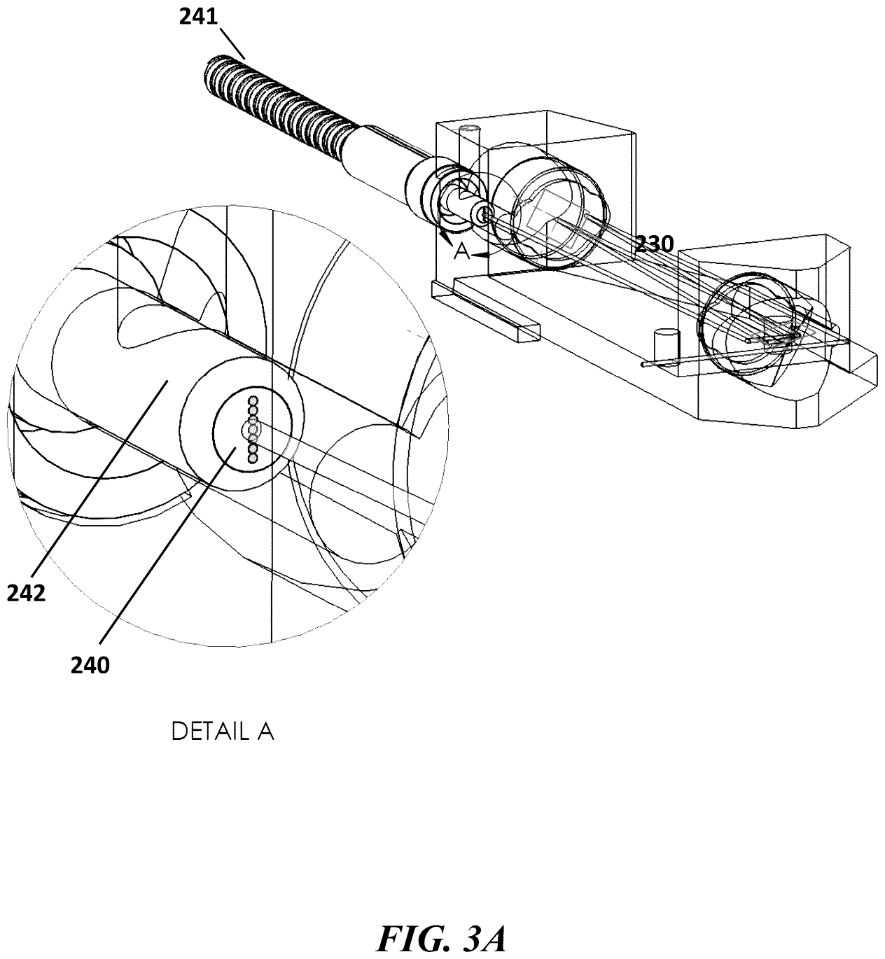

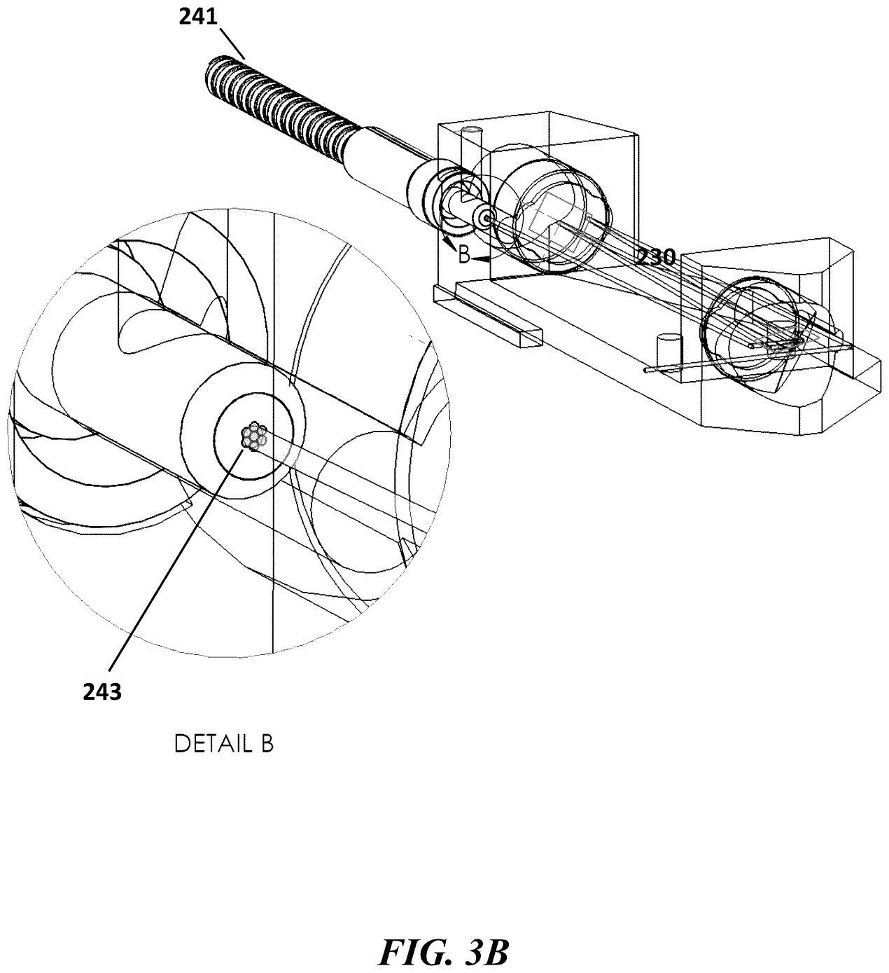

FIGS. 3A-3B illustrate examples of a collection fiber 241 (of a collector 242) or an optical fiber bundle configured to collect light at end of an optical path 230 at the output of the reflection chamber 210.

In some embodiments, the collection fiber 241 is a bundle of fibers oriented linearly (e.g., as a slit) at the output 240 of the reflection chamber as shown in FIG. 3A. In some embodiments, the collection fiber 241 is in a bundled or round shape 243 on the receiver/detector side of the collection fiber as illustrated in FIG. 3B. The fiber bundle 241 can be relatively larger than that used in confocal microscopy imaging for similar sized features to be imaged. For example, the fiber or linear fiber bundle can be about 1.5 mm in diameter or along its long dimension as opposed to 5 micrometers. The individual fibers of the bundle can be smaller, e.g. about 0.2 mm in diameter.

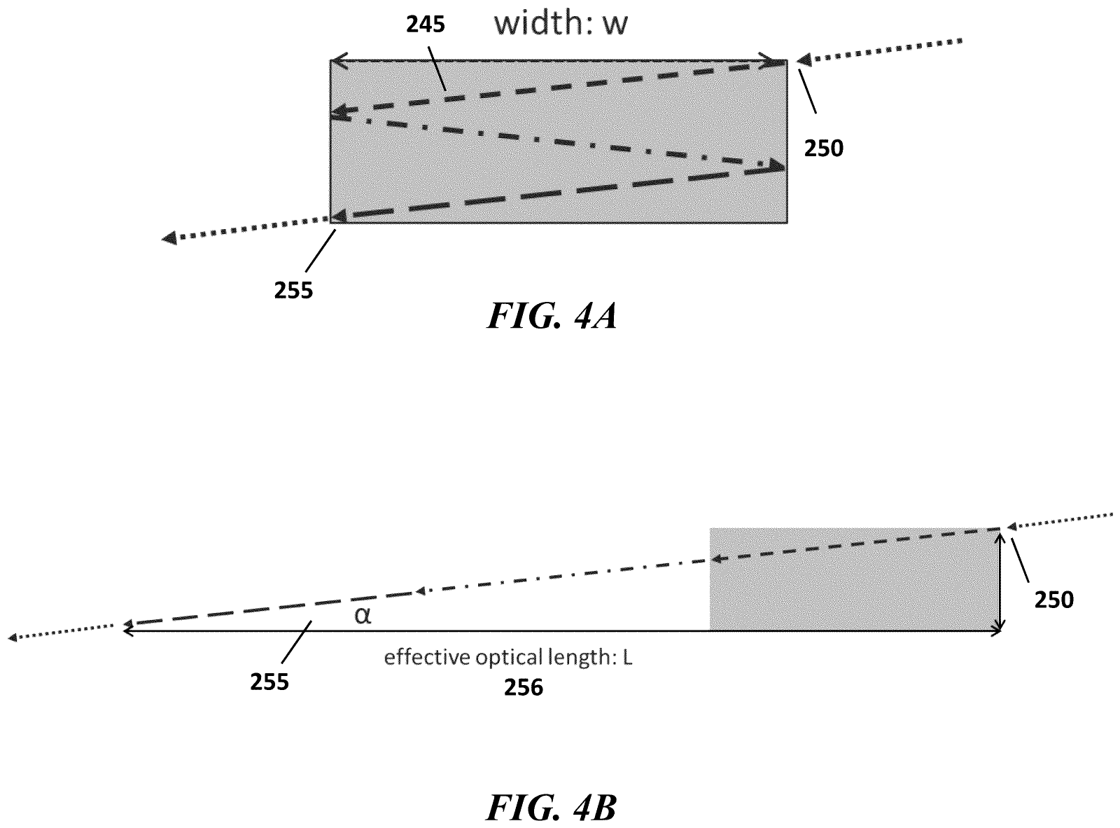

There may not be a z-component alignment used or required to focus the light on the collector as the out of focus light is filtered out along the optical path rather than being focused on a much smaller pinhole outlet. The output may have an opening with a cross section on an x and y plane that is relatively the same or relatively similar to a cross section on an x and y plane of the beam of light passing from the input to the output of the chamber. In an example, the confocal microscopy arrangement does not include an emission pinhole for light filtering. FIGS. 4A-4B schematically illustrate an optical path from the input 220 of the chamber 210 to the output 240 of the chamber 210. The optical path of light may reflect between reflectors 270, 280. As shown in FIG. 4A, the path may be folded each time the collected collimated light is reflected from a reflector, forming a plurality of path segments 245. The path segments 245 from the input, reflected between reflectors and then to the output form an optical path configured to have a length along which out of focused light is removed.

A length of a path segment 245 may be of a similar magnitude to the distance between the reflectors 270, 280. At least two path segments 245 may be formed between reflections from reflectors. The path segments may be equal to or slightly greater than a distance between the reflectors. According to some embodiments, the number of path segments 245 may be greater than or equal to 2, 3, 4, 5, 6, 8, 10, 12, 15, 20, 25, 30, 40, 50, or more. In an example, the number of path segments may be from about 2 to 3, 2 to 4, 2 to 5, 2 to 6, 2 to 8, 2 to 10, 2 to 12, 2 to 15, 2 to 20, 2 to 25, 2 to 30, 2 to 40, 2 to 50, 3 to 4, 3 to 5, 3 to 6, 3 to 8, 3 to 10, 3 to 12, 3 to 15, 3 to 20, 3 to 25, 3 to 30, 3 to 40, 3 to 50, 4 to 5, 4 to 6, 4 to 8, 4 to 10, 4 to 12, 4 to 15, 4 to 20, 4 to 25, 4 to 30, 4 to 40, 4 to 50, 5 to 6, 5 to 8, 5 to 10, 5 to 12, 5 to 15, 5 to 20, 5 to 25, 5 to 30, 5 to 40, 5 to 50, 6 to 8, 6 to 10, 6 to 12, 6 to 15, 6 to 20, 6 to 25, 6 to 30, 6 to 40, 6 to 50, 8 to 10, 8 to 12, 8 to 15, 8 to 20, 8 to 25, 8 to 30, 8 to 40, 8 to 50, 10 to 12, 10 to 15, 10 to 20, 10 to 25, 10 to 30, 10 to 40, 10 to 50, 12 to 15, 12 to 20, 12 to 25, 12 to 30, 12 to 40, 12 to 50, 15 to 20, 15 to 25, 15 to 30, 15 to 40, 15 to 50, 20 to 25, 20 to 30, 20 to 40, 20 to 50, 25 to 30, 25 to 40, 25 to 50, 30 to 40, 30 to 50, or 40 to 50. In an example, the number of path segments 245 may be greater than or equal to 3. In another example, the number of path segments may be greater than or equal to 5. In another example, the number of path segments 245 may be from about 5 to 30. According to some embodiments the number of path segments 245 is between 5 and 30. According to some embodiments the number of path segments 245 is between 3 and 50. According to some examples, the light enters into the chamber 210 is at an angle of entry 250. The entry angle 250 is approximately the same as the output angle 255. FIG. 4B illustrates the effective length L 256 of the path (e.g., pathlength). As the angle 255 approaches zero, the effective length of the hypotenuse approaches the length of the adjacent side illustrated as effective length L 256. In some cases, the effective length may describe the length of the optical path. Accordingly, the effective length L 256 is an approximation based on a small angle 255. The light reflects between the reflectors within the chamber such that the in-focus light moves along a path until it reaches the output 240. As the collimated light moves along the path, the out of focus light may diverge from the path and scatters.

The reflection chamber may include a first reflector and a second reflector. The first reflector and the second reflector may be separated by a distance. The effective length L or pathlength may be at least 2, 3, 4, 5, 6, 8, 10, 12, 15, 20, 25, 30, 40, 50, or more than the largest inner linear dimension of the chamber or the distance between the first reflector and the second reflector. The effective length L or pathlength may be from about 2 to 3, 2 to 4, 2 to 5, 2 to 6, 2 to 8, 2 to 10, 2 to 12, 2 to 15, 2 to 20, 2 to 25, 2 to 30, 2 to 40, 2 to 50, 3 to 4, 3 to 5, 3 to 6, 3 to 8, 3 to 10, 3 to 12, 3 to 15, 3 to 20, 3 to 25, 3 to 30, 3 to 40, 3 to 50, 4 to 5, 4 to 6, 4 to 8, 4 to 10, 4 to 12, 4 to 15, 4 to 20, 4 to 25, 4 to 30, 4 to 40, 4 to 50, 5 to 6, 5 to 8, 5 to 10, 5 to 12, 5 to 15, 5 to 20, 5 to 25, 5 to 30, 5 to 40, 5 to 50, 6 to 8, 6 to 10, 6 to 12, 6 to 15, 6 to 20, 6 to 25, 6 to 30, 6 to 40, 6 to 50, 8 to 10, 8 to 12, 8 to 15, 8 to 20, 8 to 25, 8 to 30, 8 to 40, 8 to 50, 10 to 12, 10 to 15, 10 to 20, 10 to 25, 10 to 30, 10 to 40, 10 to 50, 12 to 15, 12 to 20, 12 to 25, 12 to 30, 12 to 40, 12 to 50, 15 to 20, 15 to 25, 15 to 30, 15 to 40, 15 to 50, 20 to 25, 20 to 30, 20 to 40, 20 to 50, 25 to 30, 25 to 40, 25 to 50, 30 to 40, 30 to 50, or 40 to 50 of the largest inner linear dimension of the chamber or the distance between the first reflector and the second reflector. In an example, the pathlength is at least three times the largest inner linear dimension of the chamber. In another example, the pathlength is at least three times the distance between the first reflector and the second reflector. In another example, the pathlength is at least five times the largest inner linear dimension of the chamber. In another example the pathlength is at least five times the distance between the first reflector and the second reflector. In another example, the pathlength is from about 5 times to about 30 times the largest inner linear dimension of the chamber. In another example, the pathlength is from about 5 times to about 30 times the distance between the first reflector and the second reflector

The greater the effective length L, the more out of focus light may diverge or dissipate from the collimated light beam along the path. The effective length L of the collimated light beam within the chamber corresponds to the entry angle 250 (α) and the height H of the chamber. The shallower the angle α of the light entering or the greater the height H, the more reflections and the greater the effective length L. The relationship between the effective length L, height H and angle α can be represented by: L=H/α. (See, e.g., FIG. 4B). Thus, angle α or height H (e.g., the diameter of the reflector) can be selected to provide a selected effective length L. According to some embodiments, the effective length L or pathlength is greater than or equal to about 0.1, 0.2, 0.3, 0.4, 0.5 0.6, 0.7, 0.8, 0.9, 1.0, 1.5, 2, 3, 4, 5, or more meters (m). According to some embodiments, the effective length L or pathlength is less than or equal to about 5, 4, 3, 2, 1.5, 1, 0.9, 0.8, 0.7, 0.6, 0.5, 0.4, 0.3, 0.2, 0.1, or less meters. The effective length L or pathlength may be from about 0.1 m to 0.2 m, 0.1 m to 0.3 m, 0.1 m to 0.4 m, 0.1 m to 0.5 m, 0.1 m to 0.6 m, 0.1 m to 0.7 m, 0.1 m to 0.8 m, 0.1 m to 0.9 m, 0.1 m to 1 m, 0.1 m to 1.5 m, 0.1 m to 2 m, 0.1 m to 3 m, 0.1 m to 4 m, 0.1 m to 5 m, 0.2 m to 0.3 m, 0.2 m to 0.4 m, 0.2 m to 0.5 m, 0.2 m to 0.6 m, 0.2 m to 0.7 m, 0.2 m to 0.8 m, 0.2 m to 0.9 m, 0.2 m to 1 m, 0.2 m to 1.5 m, 0.2 m to 2 m, 0.2 m to 3 m, 0.2 m to 4 m, 0.2 m to 5 m, 0.3 m to 0.4 m, 0.3 m to 0.5 m, 0.3 m to 0.6 m, 0.3 m to 0.7 m, 0.3 m to 0.8 m, 0.3 m to 0.9 m, 0.3 m to 1 m, 0.3 m to 1.5 m, 0.3 m to 2 m, 0.3 m to 3 m, 0.3 m to 4 m, 0.3 m to 5 m, 0.4 m to 0.5 m, 0.4 m to 0.6 m, 0.4 m to 0.7 m, 0.4 m to 0.8 m, 0.4 m to 0.9 m, 0.4 m to 1 m, 0.4 m to 1.5 m, 0.4 m to 2 m, 0.4 m to 3 m, 0.4 m to 4 m, 0.4 m to 5 m, 0.5 m to 0.6 m, 0.5 m to 0.7 m, 0.5 m to 0.8 m, 0.5 m to 0.9 m, 0.5 m to 1 m, 0.5 m to 1.5 m, 0.5 m to 2 m, 0.5 m to 3 m, 0.5 m to 4 m, 0.5 m to 5 m, 0.6 m to 0.7 m, 0.6 m to 0.8 m, 0.6 m to 0.9 m, 0.6 m to 1 m, 0.6 m to 1.5 m, 0.6 m to 2 m, 0.6 m to 3 m, 0.6 m to 4 m, 0.6 m to 5 m, 0.7 m to 0.8 m, 0.7 m to 0.9 m, 0.7 m to 1 m, 0.7 m to 1.5 m, 0.7 m to 2 m, 0.7 m to 3 m, 0.7 m to 4 m, 0.7 m to 5 m, 0.8 m to 0.9 m, 0.8 m to 1 m, 0.8 m to 1.5 m, 0.8 m to 2 m, 0.8 m to 3 m, 0.8 m to 4 m, 0.8 m to 5 m, 0.9 m to 1 m, 0.9 m to 1.5 m, 0.9 m to 2 m, 0.9 m to 3 m, 0.9 m to 4 m, 0.9 m to 5 m, 1 m to 1.5 m, 1 m to 2 m, 1 m to 3 m, 1 m to 4 m, 1 m to 5 m, 1.5 m to 2 m, 1.5 m to 3 m, 1.5 m to 4 m, 1.5 m to 5 m, 2 m to 3 m, 2 m to 4 m, 2 m to 5 m, 3 m to 4 m, or 4 m to 5 m. According to some embodiments, the effective length L or pathlength is from about 0.3 m to 1.5 m. In an example, the effective length L or pathlength is greater than about 0.5 m. In an example, the effective length L or pathlength is greater than or equal to about 1 m. In another example, the effective length L or pathlength is greater than or equal to about 1.5 m.

According to some embodiments, the filtering device 200 and reflection chamber 210 are configured or sized to fit within a handheld device. In addition to selecting the height H, angle α and the selected effective length L, the width W of the chamber can also be selected. While the effective length L or pathlength is dependent on the angle α and the height H of the chamber, it may be independent of the width. Thus, shortening the width may permit compressing the width of the chamber to enable fitting the chamber within a handheld device without changing the effective length, L. However, some light (e.g., power) may be lost with each reflection thus limiting the degree of widthwise chamber compression to optimize resolution of an image. According to some embodiments the width of the chamber may be from about 10 to 200 mm. In an example, a distance separating reflectors (e.g., a distance between a first location and a second location in the chamber) may be greater than or equal to about 1 centimeter (cm), 2 cm, 3 cm, 4 cm, 5 cm, 6 cm, 7 cm, 8 cm, 9 cm, 10 cm, 12 cm, 15 cm, 20 cm, or more. In another example, a distance separating reflectors (e.g., a distance between a first location and a second location in the chamber) may be less than or equal to about 20 cm, 15 cm, 12 cm, 10 cm, 9 cm, 8 cm, 7 cm, 6 cm, 5 cm, 4 cm, 3 cm, 2 cm, 1 cm, or less. In another example, a largest inner linear dimension of the chamber may be from about 1 cm to 2 cm, 1 cm to 3 cm, 1 cm to 4 cm, 1 cm to 5 cm, 1 cm to 6 cm, 1 cm to 7 cm, 1 cm to 8 cm, 1 cm to 9 cm, 1 cm to 10 cm, 1 cm to 12 cm, 1 cm to 15 cm, 1 cm to 20 cm, 2 cm to 3 cm, 2 cm to 4 cm, 2 cm to 5 cm, 2 cm to 6 cm, 2 cm to 7 cm, 2 cm to 8 cm, 2 cm to 9 cm, 2 cm to 10 cm, 2 cm to 12 cm, 2 cm to 15 cm, 2 cm to 20 cm, 3 cm to 4 cm, 3 cm to 5 cm, 3 cm to 6 cm, 3 cm to 7 cm, 3 cm to 8 cm, 3 cm to 9 cm, 3 cm to 10 cm, 3 cm to 12 cm, 3 cm to 15 cm, 3 cm to 20 cm, 4 cm to 5 cm, 4 cm to 6 cm, 4 cm to 7 cm, 4 cm to 8 cm, 4 cm to 9 cm, 4 cm to 10 cm, 4 cm to 12 cm, 4 cm to 15 cm, 4 cm to 20 cm, 5 cm to 6 cm, 5 cm to 7 cm, 5 cm to 8 cm, 5 cm to 9 cm, 5 cm to 10 cm, 5 cm to 12 cm, 5 cm to 15 cm, 5 cm to 20 cm, 6 cm to 7 cm, 6 cm to 8 cm, 6 cm to 9 cm, 6 cm to 10 cm, 6 cm to 12 cm, 6 cm to 15 cm, 6 cm to 20 cm, 7 cm to 8 cm, 7 cm to 9 cm, 7 cm to 10 cm, 7 cm to 12 cm, 7 cm to 15 cm, 7 cm to 20 cm, 8 cm to 9 cm, 8 cm to 10 cm, 8 cm to 12 cm, 8 cm to 15 cm, 8 cm to 20 cm, 9 cm to 10 cm, 9 cm to 12 cm, 9 cm to 15 cm, 9 cm to 20 cm, 10 cm to 12 cm, 10 cm to 15 cm, 10 cm to 20 cm, 12 cm to 15 cm, 12 cm to 20 cm, or 15 cm to 20 cm. In an example, a largest inner linear dimension of the chamber is less than or equal to 15 cm. In another example, a largest inner linear dimension of the chamber is less than or equal to 10 cm.

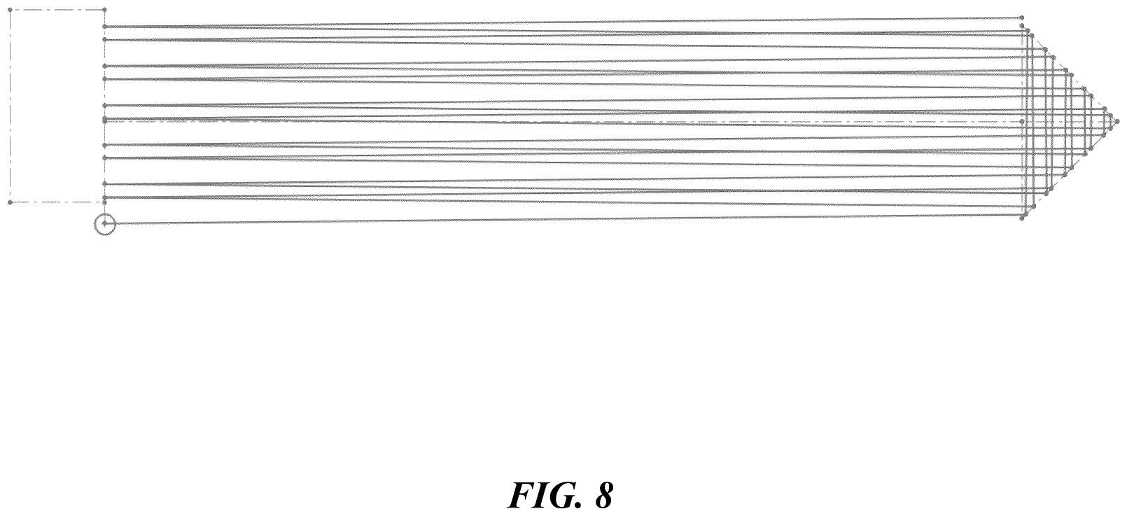

As shown in FIG. 2A, the reflectors, may be parallel and interfacing. Alternatively, or in addition to, the reflectors may be off set. According to some embodiments, at least one of the reflectors may comprise a retroreflector that has a property of reflecting light in a path parallel to the path in which the light was received at the retroreflector. According to some embodiments, at least one of the reflector may comprise a mirror. FIG. 2A illustrates reflectors 270, 280 that are a combination of a retroreflector 280 and a mirror 270, respectively used in the reflection chamber 210. In addition, FIG. 2A is a schematic illustration of an example optical path 230 using a retroreflector 280 and a mirror 270 respectively as reflectors 270, 280. An additional example of an optical path of FIG. 2A is further illustrated in FIG. 8. The optical properties of the retroreflector 280 permit the light to return in a direction parallel to incoming light, which may provide correction for misalignment between opposing reflectors by returning the light at a parallel orientation to the incoming light. FIG. 2B schematically illustrates reflection chamber 210 using mirrors 270 as the reflectors 270, creating a schematically illustrated optical path. FIG. 2C schematically illustrates a reflection chamber 210 using opposing parallel retroreflectors 280 as reflectors 280 creating a schematically illustrated optical path. The retroreflector 280 in combination with a mirror 270, as shown in FIG. 2A approximately doubles the effective length L or pathlength in comparison to the two retroreflector element path illustrated in FIG. 2C. The mirror and retroreflector combination have a fewer number of reflections for a given path length L, in comparison to the two retroreflectors element path shown in FIG. 2C.

The mirror reflector 270 within the reflection chamber 210 may be flat and may have a dielectric coating that preferentially reflects a select wavelength or range of wavelengths. According to one example the reflective coating may preferentially reflect a wavelength in the range of about 700 nm to 900 nm. The coating may provide some filtering effect to block some ambient light, particularly with multiple reflections within the path. The retroreflector may be for example, a corner cube retroreflector. The retroreflector may be configured for total internal reflection of the light. The retroreflector may be coated with a gold plating or other coating that may provide reflection properties such as low attenuation or absorption, or high reflectivity and may provide some preferential wavelength reflectivity (e.g., from about 700 nm to 900 nm).

As shown in FIG. 9, light filtering devices with a plurality of reflective elements are illustrated. A plurality of interfacing reflector arrangements (270a, 280a) and (270b, 280b) are shown in series. The collected light 250 follows a path. Some embodiments can include non-parallel orientations, or a combination of reflector orientations.

According to some embodiments, a probe may comprise a light filtering device or chamber as described herein. According to some embodiments, the light filtering device or chamber can be configured to fit or sized to fit within a handheld optical device comprising the probe.

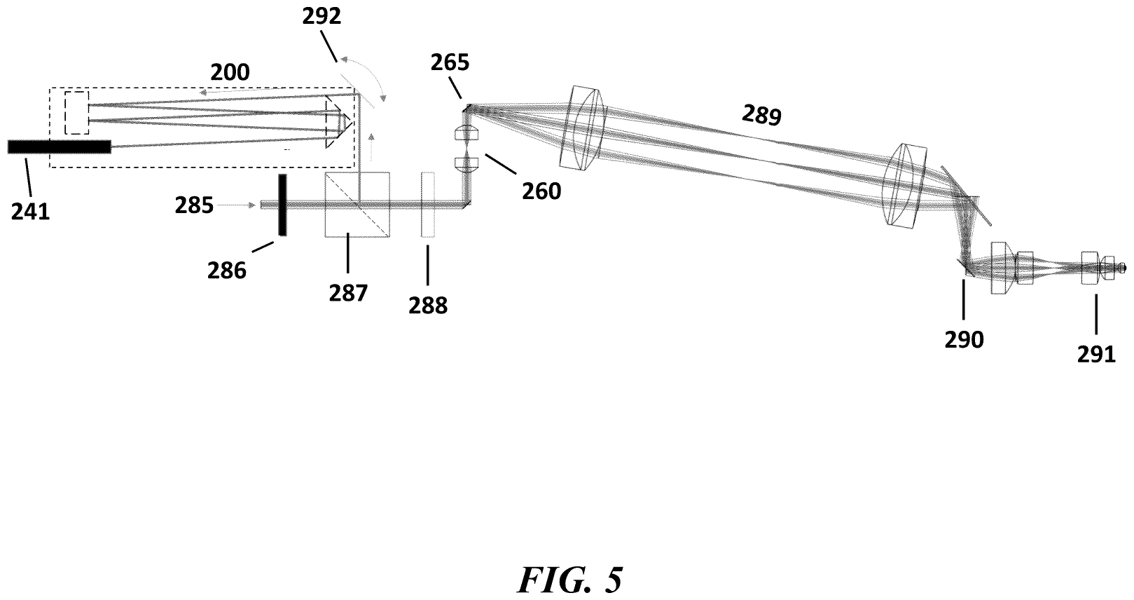

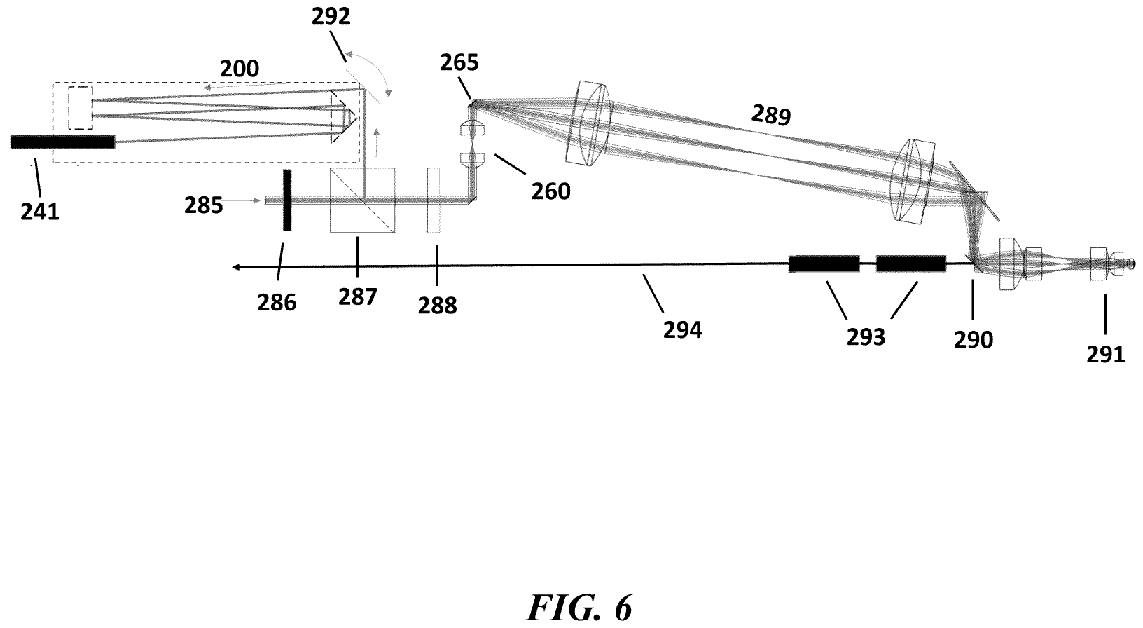

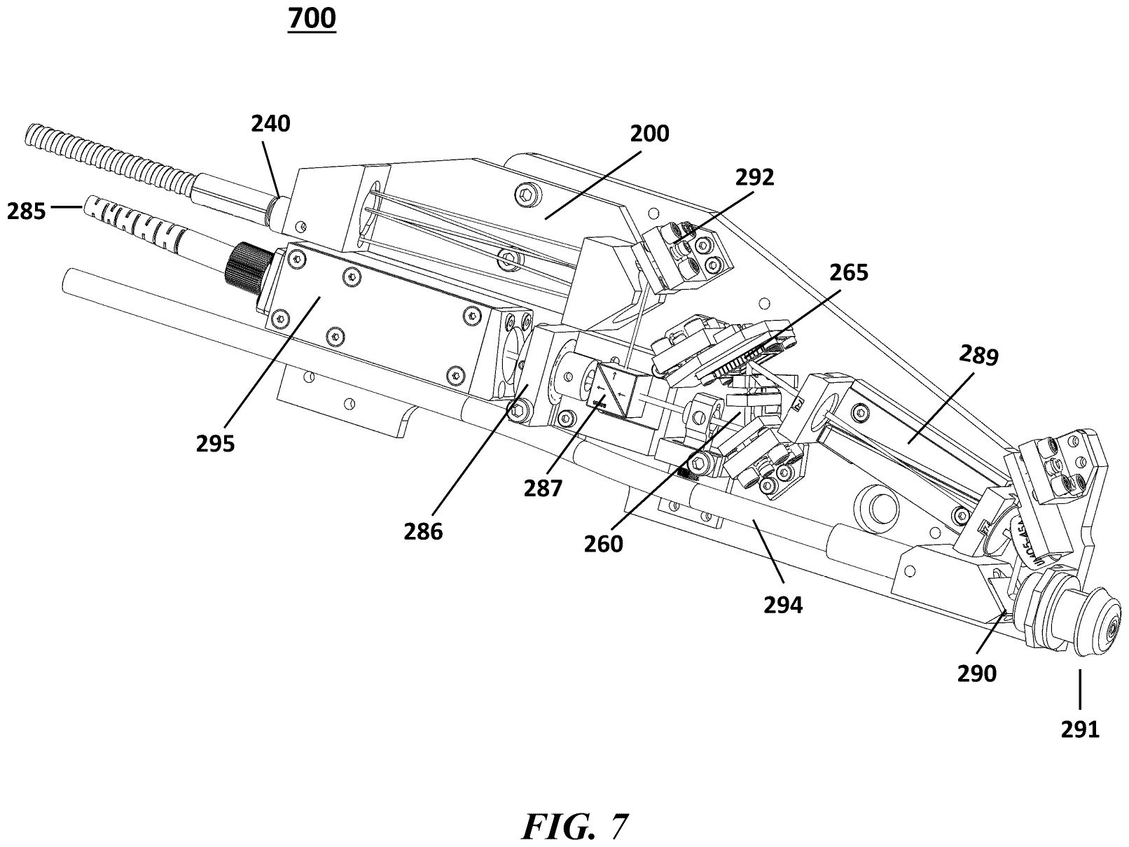

FIG. 5 shows an example of focusing and scanning optics of an optical device (e.g., a handheld device) using a light filtering device 200 as described with reference to FIGS. 1A to 4B. The light filtering device 200 may be included with one or more focusing 260 and scanning optics 265. According to some embodiments, the focusing units 260 of an optical device can be used for scanning and creating depth profiles of an imaging subject (e.g., tissue). The optical device may further comprise additional optical elements, such as a light source 285 (e.g., laser), waveplate 286 (e.g., half waveplate), beam-splitter 287 (e.g., polarized or non-polarized), alignment window 288, one or more relay lenses 289, dichroic mirror 290, probe 291, alignment unit 292, or any combination thereof. FIGS. 5 and 6 show an example of focusing units 260 of an optical device configured to simultaneously adjust a depth and a position of a focal point of an excitation light beam. FIG. 6 shows the one or more focusing 260 and scanning 265 optics of FIG. 5 with additional light collection optics 293 for collecting additional signals such as, for example, multiphoton and SHG signals, that can be used to layer images or depth profiles of imaged tissue. FIG. 7 shows examples of focusing 260 and scanning 265 components or units of the optical device of FIG. 5 or 6 positioned in a handle 700 of a handheld optical device. In an example, the optical device may be configured for confocal imaging. In another example, the optical device may be configured for multiphoton imaging. In another example, the optical device may be configured for tandem confocal and multiphoton imaging.

Referring to FIGS. 5, 6 and 7, a confocal imaging arrangement as described herein may be an optical device including a fiber optic 285 configured to transmit light from a laser to the optical device. The fiber optic 285 may be a single mode fiber, a multi-mode fiber, or a bundle of fibers. The fiber optic 285 may be a bundle of fibers configured to transmit light from multiple lasers or light sources to the optical device that are either pulsed or continuous beams. The fiber optic 285 may be coupled to a frequency multiplier 295 that converts the frequency to a predetermined excitation frequency (e.g., by multiplying the frequency by a factor of 1 or more). The frequency multiplier 295 may transmit light from fiber optic 285 to a half wave plate 286. The excitation light may be directed through a half wave plate 286 to shift the polarization direction of the light along an axis of the half wave plate 286. The light may be sent through the beam splitter 287 that directs a portion of the excitation light to a power monitor. Other sensors may be included with the probe as well as a power monitor. The sensors and monitors may provide additional information concerning the imaging device or the imaging subject (e.g., specimen) that can be included as data with the depth profile.

The illumination or excitation light may then be directed to the afocal z-axis scanner 260. The afocal z-axis scanner 260 may comprise a moveable lens and an actuator (e.g., a voice coil) coupled to the moveable lens. The afocal z-axis scanner (e.g., focusing unit) may be disposed in the optical device between the light source and the probe. The afocal z-axis scanner 260 may converge or diverge the collimated beam of light, moving the focal point in the axial direction while imaging. Moving the focal point in the axial direction may enable imaging a depth profile. The illumination or excitation light may then be directed to a scanning unit 265 (e.g., MEMS mirror). The scanning unit may be disposed in the optical device between the light source and the probe. The MEMS mirror 265 can enable scanning by moving the focal point on a horizonal plane or an X-Y plane. According to some representative embodiments, the afocal Z-scanner 260 and the MEMS mirror 265 may be separately actuated with actuators that are driven by a coordinated computer control so that their movements are synchronized to provide synchronized movement of focal points within tissue. According to some representative embodiments, moving both the moveable lens and the MEMS mirror 265 may allow changing an angle between a focal plane and an optical axis, and enable imaging a depth profile through a plane (e.g., a slanted plane or focal plane as defined herein).

The MEMS mirror scanner 265 may be configured to direct at least a part of the light through one or more relay lenses 289. The one or more relay lenses 289 may be configured to direct the light to a dichroic mirror 290. The dichroic mirror 290 may direct the excitation light into an probe 291 which may include one or more objectives. The probe 291 may be configured to direct the light to interact with an imaging subject (e.g., tissue of a subject). The probe 291 may be configured to collect one or more signals generated by the light interacting with the imaging subject, including, but not limited to, reflected light. One or more of the signals collected by the probe 291 may comprise reflected, transmitted, fluorescent/autofluorescent signals, or any combination thereof generated by light interacting with the imaging subject. A subset of one or more signals collected by the probe 291 may comprise single-photon or multi-photon generated signals.

A signal collected by an objective of the probe 291, may trace a reverse path of the light that generated the signal. In some embodiments, a returned signal may include an RCM or fluorescent/autofluorescent single photon signal. Returned light may pass through the beam splitter 287 where at least a portion of the returned light is directed to a light filtering device 200. The signal may contact an alignment unit 292, which may direct to light to a light filtering device 200. The light filtering device 200 may be coupled to collector 242. An alignment unit 292 may comprise, for example, a mirror 270 that moves light in an x- and y-axis and directs light through the input 220 of into the reflection chamber 210. The collector 242 may comprise a fiber optic such as a fiber bundle, for example, as described with reference to FIGS. 3A and 3B, configured to collect collimated light reflected in a light path through the reflection chamber 210.

The beam splitter 287 may be a polarizing selective beam splitter. Accordingly, returned off-axis, rotated or shifted polarized light components can be directed by the beam splitter 287 to the filtering device 200. The rotated reflected light can represent reflection from birefringent objects, structures, features or molecules within the imaging subject (e.g., imaged tissue). For example, structures, cells types, pigments in the epidermis, connective tissue and dermis of clinical imaging significance, can be birefringent and rotate the polarity of the light. The specularly reflected light may be predominant and brighter as compared to the rotated light, follows its original path and returns through the beam splitter 287. The polarizing beam splitter 287 may thereby operate to select the light corresponding to structures that have rotated the light and direct that light to the alignment unit 292 and then into a reflection chamber 210. The specularly reflected light can be directed through a separate channel to a different reflection chamber or may be allowed to dissipate as it returns back through the beam splitter 287. The reflected signals can be further split through additional separate channels based on polarization or wavelength and into different reflection chambers for processing.

The alignment unit 292 may be configured to move the light 250 signal split off by the beamsplitter, in the x- and y-axes to direct the light at a desirable angle α through the input 220 and into the reflection chamber 210. The light filtering device 200 may be configured so that further focusing of the light directed from the beam splitter 287 onto an optical fiber is not used or so that z-direction positioning of the light focal point is not used. According to some embodiments herein, the x- and y-axes positioning or angle of the light entering the opening 220 can be selected by observing the light or image from the fiber optic 241 output. The optical fiber 241 may be a single mode fiber, a multimode fiber, or a bundle of fibers. The optical fiber 241 may be coupled to a photodetector for detecting the reflected signal.

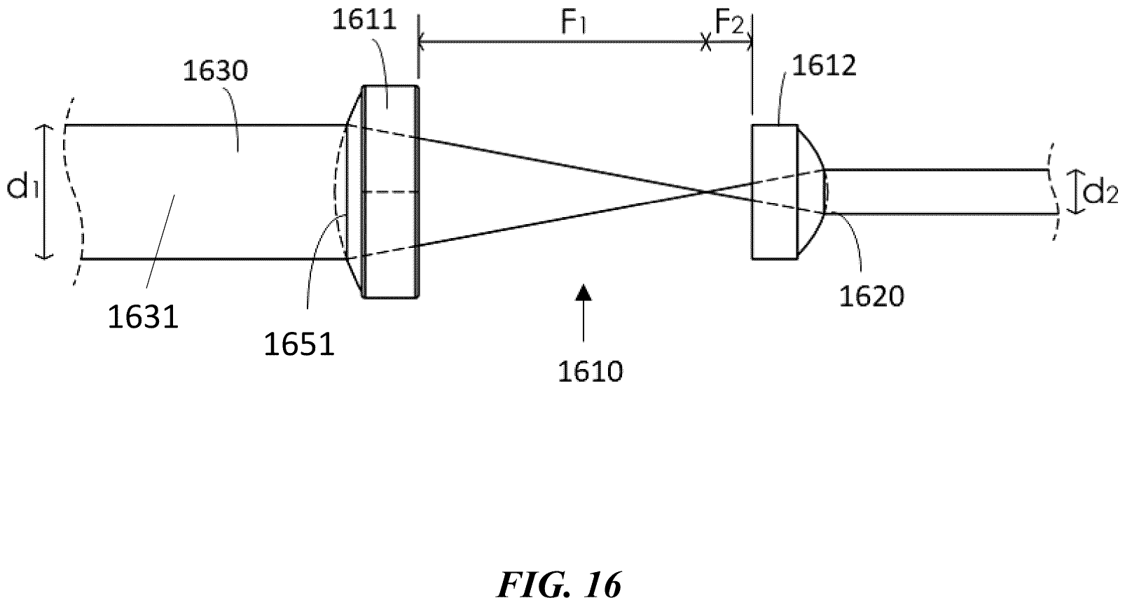

Referring to FIGS. 16-18, a beam de-expander unit may be added to a light path before it reaches a light filtering device described in FIGS. 1-9 herein. The beam de-expander unit may comprise one or more lenses (e.g., a first lens and a second lens). The beam de-expander unit may comprise a chamber (e.g., a chamber of the beam de-expander unit). One or more walls of the chamber of the beam de-expander unit may be deformable walls (e.g., deformable inward or deformable outward). One or more walls of the chamber of the beam de-expander unit may not be deformable walls. The beam de-expander unit may comprise a tuning structure. The tuning structure may be configured to deform the deformable walls of the chamber of the de-expander. Accordingly, the rejection of off-axis light may be further amplified as it passes through the beam de-expander unit prior to entering a chamber of the light filtering device described herein. In addition, the beam de-expander unit may de-expand or reduce a beam diameter of the light path.

According to some embodiments, the beam de-expander unit may comprise a chamber (e.g., a chamber of the beam de-expander unit). The beam de-expander (e.g., the beam de-expander unit) may comprise two lenses (e.g., a first lens and a second lens) positioned on opposing ends of the chamber of the beam de-expander unit.

The beam de-expander (e.g., the beam de-expander unit) may be provided with a tuning structure to allow for precise spacing between the lenses so that light that is output from the beam de-expander (e.g., the beam de-expander unit) is a collimated beam of in-focus light.

In some embodiments, the tuning structure (e.g., tuning unit, tuning structure unit, tuning device, tuning structure device, tuning mechanism) may comprise one or more screws (e.g., a first screw and a second screw), a press, an actuator, a beam profiler, one or more jaws (e.g., a first jaw and a second jaw), one or more die elements (e.g., a first die element and a second die element), one or more block elements (e.g., a first block element and a second block element), a knob, a lever, or combinations thereof. In some embodiments, the tuning structure may allow for precise spacing between the first lens and the second lens using tuning structure elements such one or more screws (e.g., a first screw and a second screw), a press, an actuator, a beam profiler, one or more jaws (e.g., a first jaw and a second jaw), one or more die elements (e.g., a first die element and a second die element), one or more block elements (e.g., a first block element and a second block element), a knob, a lever, or combinations thereof. According to some embodiments, the tuning structure may be configured to adjust a distance between the lenses.

In some embodiments, to adjust the distance between the lenses, a tuning structure element is actuated to deform a wall or walls of the chamber in one or more locations between the lenses. When actuated, tuning structure elements engage and deform walls of the chamber to reduce the length of the chamber and thereby decrease the distance between the lenses. According to some embodiments the deformation is uniform or symmetric to provide a uniform change in the distance between the lenses. According to some embodiments, the actuator may actuate elements or features that rotate screws that move the walls of the chamber outwardly (or inwardly). The screws may engage screw threads in the chamber wall. The screws may be positioned perpendicularly to the light path through the chamber. In other embodiments, to adjust the distance between the lenses, the actuator may drive the press towards the second jaw, which may drive the block elements to symmetrically compress and deform the chamber of the beam de-expander walls inwardly. The deformation of the chamber of the beam de-expander walls may decrease the distance between the lenses. In some embodiments, the distance between the lenses may be about 1 millimeters (mm) to about 150 mm. In some embodiments, the distance between the lenses may be greater than or equal to about 1 mm, 2 mm, 5 mm, 7 mm, 8 mm, 9 mm, 10 mm, 11 mm, 12 mm, 13 mm, 15 mm, 20 mm, 25 mm, 50 mm, 75 mm, 100 mm, 150 mm. In some embodiments, the distance between the lenses may be less than or equal to about 1 mm, 2 mm, 5 mm, 7 mm, 8 mm, 9 mm, 10 mm, 11 mm, 12 mm, 13 mm, 15 mm, 20 mm, 25 mm, 50 mm, 75 mm, 100 mm, or 150 mm. In some embodiments, the distance between the lenses may be between any two values described above, for example between about 5 mm and about 15 mm.

According to some embodiments, the chamber of the beam de-expander may be deformable. For example, one or more walls of the chamber of the beam de-expander may be deformable to decrease or increase a distance between opposing ends of the chamber (i.e., where the lenses are located on opposing ends of the chamber). Alternatively, or additionally, for example, one or more walls of the chamber of the beam de-expander may be deformable to increase or decrease a distance between a first wall and a second wall of the one or more walls of the chamber of the beam de-expander. According to some embodiments, the chamber (e.g., a wall of the chamber) of the beam de-expander may be inwardly deformable. Alternatively, or in addition, the chamber (e.g., a wall of the chamber) of the beam de-expander may be outwardly deformable.

A light beam enters and passes through the beam de-expander (e.g., the chamber of the beam de-expander). A collimated light exits the beam de-expander and then may be directed to a light filtering device comprising a reflection chamber (e.g., a chamber of the light filtering device as described with reference to FIGS. 1-9 herein). The light filtering device may comprise a collection fiber. The collection fiber may be configured to collect light at the end of the chamber (e.g., the chamber of the light filtering device).

FIG. 16 schematically illustrates an example of a light beam path 1631 through a beam de-expander 1610 (e.g., the beam de-expander unit). Collected or returned light (which may include a collimated beam 1630) may follow the light beam path 1631 by entering and passing through the beam de-expander 1610 before it reaches a light filtering device described in FIGS. 1-9 herein. The beam de-expander 1610 may comprise a first lens 1611 and a second lens 1612. The first lens 1611 and the second lens 1612 may comprise a first focal length F1 and a second focal length F2, respectively. In some embodiments, focal length F1 and focal length F2 may be different. In some embodiments, the distance between the lenses 1611, 1612 may be the sum of the focal lengths F1, F2 of the lenses 1611, 1612, respectively.

In some embodiments, the collected light beam input into the de-expander 1610 may comprise returned light including in-focus light as described herein with reference to FIGS. 1-9.

Collected light comprising a collimated light beam 1630 may be directed into the beam de-expander (e.g., the beam de-expander unit) 1610 through an input 1615 of the beam de-expander 1610. The collected light may enter the input 1615 through the first lens 1611 of the beam de-expander 1610 by which the collected light may be focused, but where a divergence or a convergence of the off-axis light is amplified. The second lens 1612, which may have a shorter focal length than the first lens 1611, is positioned so that the collimated beam 1630 that enters the beam de-expander is collimated when exiting an output 1620 of the beam de-expander 1610 (e.g., the beam de-expander unit). The collimated beam 1630 may comprise a diameter d1 before entering the input 1615 of the beam de-expander 1610. The collimated beam 1630 may comprise a diameter d2 after exiting the output 1620 of the beam de-expander 1610. As shown in FIG. 16, the beam de-expander 1610 (e.g., the beam de-expander unit) may reduce the diameter d1 of the collimated beam 1630 prior to entering the input 1615 to the diameter d2 of the collimated beam 1630 subsequent to exiting the output 1620 while amplifying rejection of off-axis light. In some embodiments, the diameter d1 is greater than the diameter d2. In some embodiments, the beam de-expander 1610 may comprise two positive focal length lenses. In some embodiments, the beam de-expander 1610 may comprise two negative focal length lenses. Alternatively, or in addition, the beam de-expander 1610 may comprise a combination of positive and negative focal length lenses.

The beam de-expander (e.g., the beam de-expander unit) 1610 may comprise greater than or equal to two lenses. The beam de-expander 1610 may comprise greater than or equal to three lenses, greater than or equal to four lenses, greater than or equal to five lenses, greater than or equal to six lenses, greater than or equal to seven lenses, greater than or equal to eight lenses, greater than or equal to nine lenses, or greater than or equal to ten lenses. The beam de-expander (e.g., the beam de-expander unit) 1610 may comprise less than or equal to two lenses. The beam de-expander 1610 may comprise less than or equal to three lenses, less than or equal to four lenses, less than or equal to five lenses, less than or equal to six lenses, less than or equal to seven lenses, less than or equal to eight lenses, less than or equal to nine lenses, or less than or equal to ten lenses. The beam de-expander 1610 may (e.g., the chamber of the beam de-expander) alternatively or additionally comprise curved mirrors.

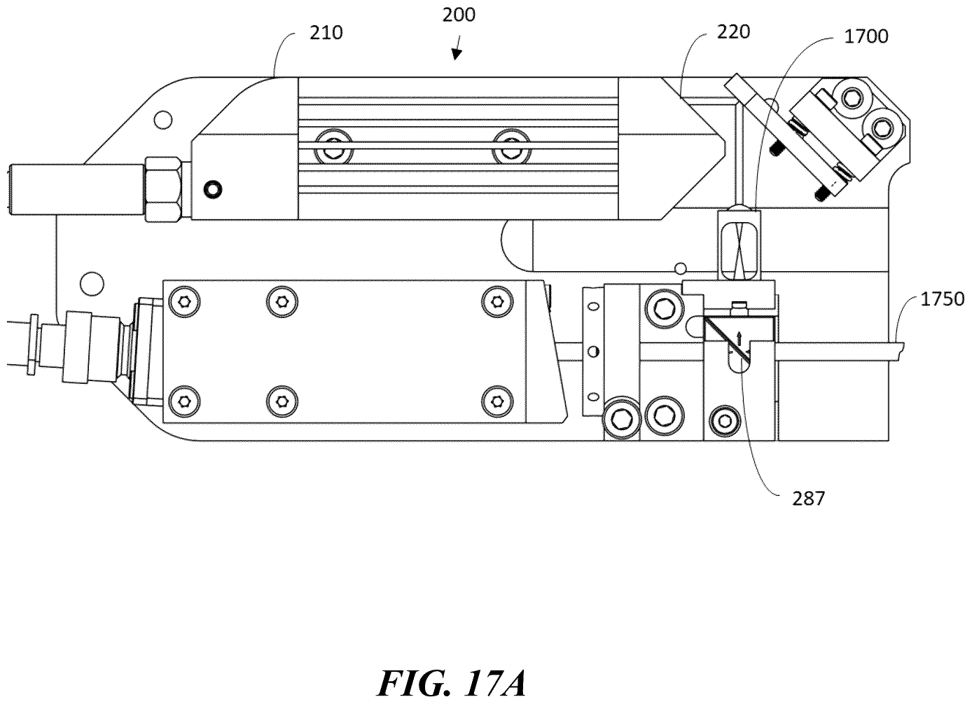

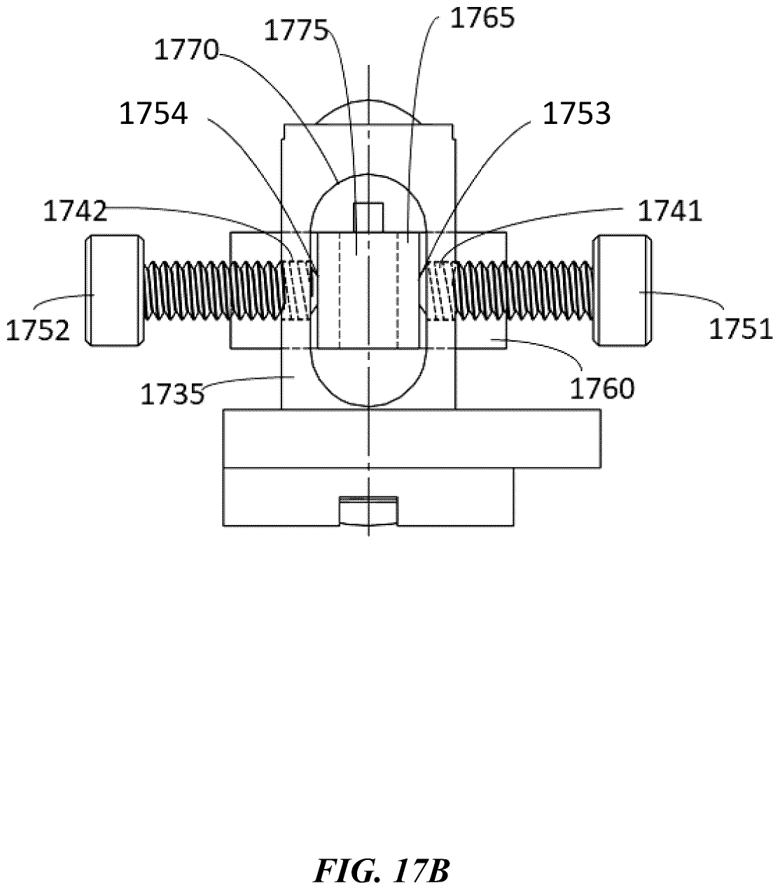

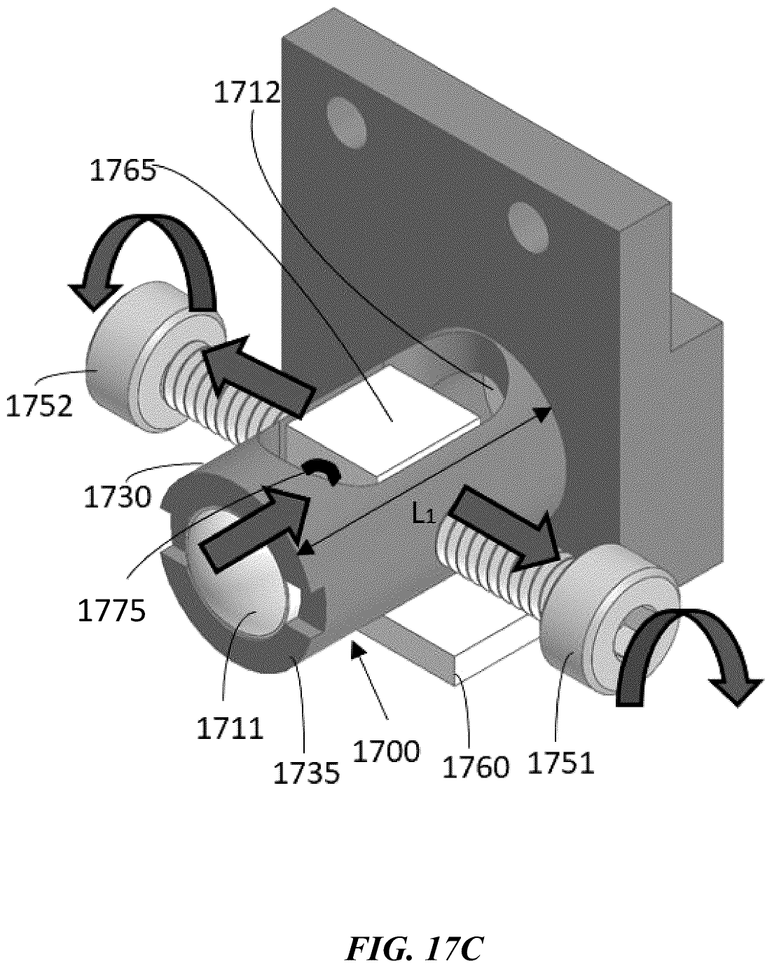

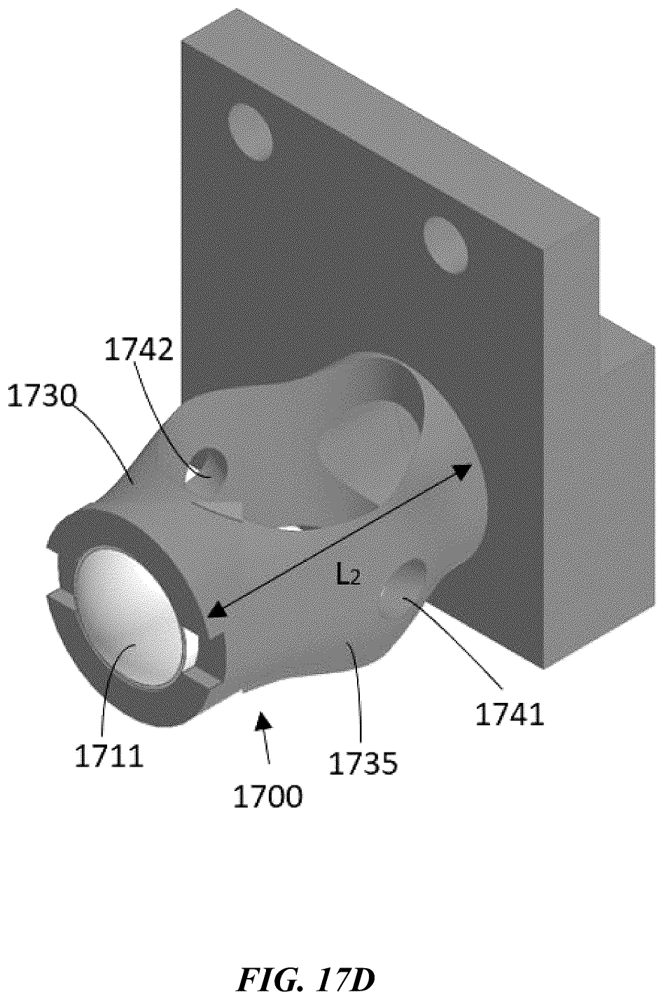

Referring to FIGS. 17A-17D, a beam de-expander (e.g., the beam de-expander unit) 1700 may be positioned between a collected light beam (e.g., returned light) 1750 and an input 220 of a light filtering device 200 that may lead into a chamber 210 of the light filtering device 200 described with reference to FIGS. 1-9 herein. As shown in FIG. 17A, the collected light beam 1750 may pass through a beam splitter 287 where at least a portion of the returned light may pass through the beam de-expander 1700 prior to being directed to the input 220 of the light filtering device 200. The collected light beam 1750 may include a collimated beam before entering the beam de-expander 1700 The collected light beam 1750 may comprise a collimated beam after exiting the beam de-expander 1700.

Referring to FIGS. 17A to 17D, the beam de-expander (e.g., the beam de-expander unit) 1700 may comprise lenses 1711, 1712 mounted at opposite ends of a chamber of the beam de-expander 1730. The collected light beam 1750 may be collected by a probe or an imaging device described herein in reference to FIGS. 1-9. The collected light beam 1750 may pass through the lenses 1711, 1712 and the chamber of the beam de-expander 1730. The chamber of the beam de-expander 1730 may comprise at least one wall structure 1735. The chamber of the beam de-expander 1730 may comprise at least two, at least three, at least four, at least five, at least six, at least seven, at least eight, at least nine, or at least ten wall structures 1735.

The wall structure 1735 may be deformable. In some embodiments, the wall structure 1735 may be outwardly deformable. In some embodiments, the wall structure 1735 may be inwardly deformable. Alternatively, or in addition, in some embodiments, the wall structure 1735 may be inwardly and outwardly deformable. In some embodiment, the wall structure 1735 may be outwardly deformable to reduce the distance between the lenses 1711, 1712 (e.g., to reduce the length of the chamber of the beam de-expander 1730 between the first lens 1711 and the second lens 1712 from a first length L1 (as shown in FIG. 17C) to a second length L2 (as shown in FIG. 17D)).

Referring to FIGS. 17B and 17D, the wall structure 1735 of the chamber of the beam de-expander 1730 may comprise threaded openings 1741, 1742. The threaded openings may comprise an orientation of opposing threaded openings 1741, 1742. The opposing threaded openings 1741, 1742 may or may not be perpendicular to an axis between the two lenses of the chamber of the beam de-expander 1730 (e.g., the axis may correspond to a light path of a beam passing through a first lens 1711, through the chamber of the de-expander 1730, and out through the second lens 1712). The opposing threaded openings 1741, 1742 may receive screws 1751, 1752 (as shown in FIGS. 17B and 17C), respectively. The screws 1751, 1752 may be rotated to buckle the wall structure 1735 of the chamber of the beam de-expander 1730 outwardly. Alternatively, or in addition, the screws 1751, 1752 may be rotated to buckle the wall structure 1735 of the chamber of the beam de-expander 1730 inwardly. The screws 1751, 1752 may pull the first lens 1711 and the second lens 1712 towards each other to shorten the first length L1 to a second length L2 of the chamber of the beam de-expander 1730.

Referring to FIG. 17B, a tuning structure (e.g., tuning unit, tuning structure unit, tuning device, tuning structure device, tuning mechanism) may comprise a T-shaped structure 1760. The T-shaped structure may comprise a block portion 1765 that may be inserted into an opening 1770 of the wall structure 1735. The block portion 1765 may extend into the chamber of the beam de-expander 1730 and may be positioned between the first lens 1711 and the second lens 1712. The block portion 1765 may comprise an opening 1775 that may align with the light path 1631 of the beam. The opening 1775 of the block portion 1765 may permit the light beam to pass through the block portion 1765. The block portion 1765 may engage a first end 1753 of a screw 1751 and a second end 1754 of a screw 1752 as the screws 1751, 1752 are turned. The turning of the screws 1751, 1752 may symmetrically regulate the deformation of the chamber of the beam de-expander 1730 wall structure 1735.

The chamber of the beam de-expander 1730 may comprise a deformable rigid material. The deformable rigid material may maintain the shape of the chamber of the bean de-expander 1730 when deformed. The deformable rigid material may comprise one or more metals such as stainless steel, aluminum, or any other metal or material that may maintain a rigid deformable shape.

Referring to FIG. 17C, a distance between the first lens 1711 and the second lens 1712 may be initially set (e.g., during manufacturing) slightly greater than a desired distance. For example, a desired distance may be the sum of the focal lengths F1, F2, respectively of the lenses 1711, 1712. In other embodiments, the initial distance between the first lens 1711 and the second lens 1712 may be set slightly less than or the same as a distance that comprises the sum of the focal lengths of the lenses 1711, 1712.

The beam de-expander (e.g., the beam de-expander unit) 1700 may be tuned (e.g., by the tuning structure). The tuning structure (e.g., tuning unit, tuning structure unit, tuning device, tuning structure device, tuning mechanism) may adjust the spacing between the first lens 1711 and the second lens 1712 to provide for a collimated beam of in-focus light to the input of the light filtering device 200, and subsequently at the location of a collection fiber or a collector at the end of a reflection chamber 210 of a light filtering device 200 (as shown in FIG. 17A).

To tune the beam de-expander 1700, the tuning structure may be used to determine a beam diameter. In some embodiments, the tuning structure may be used to determine a small beam diameter. According to some embodiments a beam profiler may be used to determine the smaller beam diameter. As shown in FIG. 17C, the beam diameter may be monitored by adjusting the screws 1751, 1752. The beam diameter may indicate an amplified rejection of off-axis light (e.g., a smaller beam diameter may indicate a greater amplified rejection of off-axis light). When the beam de-expander (e.g., the beam de-expander unit) 1700 is tuned by the tuning structure, the elements of the tuning structure, (e.g., the screws 1751, 1752 and the T-shaped structure 1760) may or may not be removed (as shown in FIG. 17D).

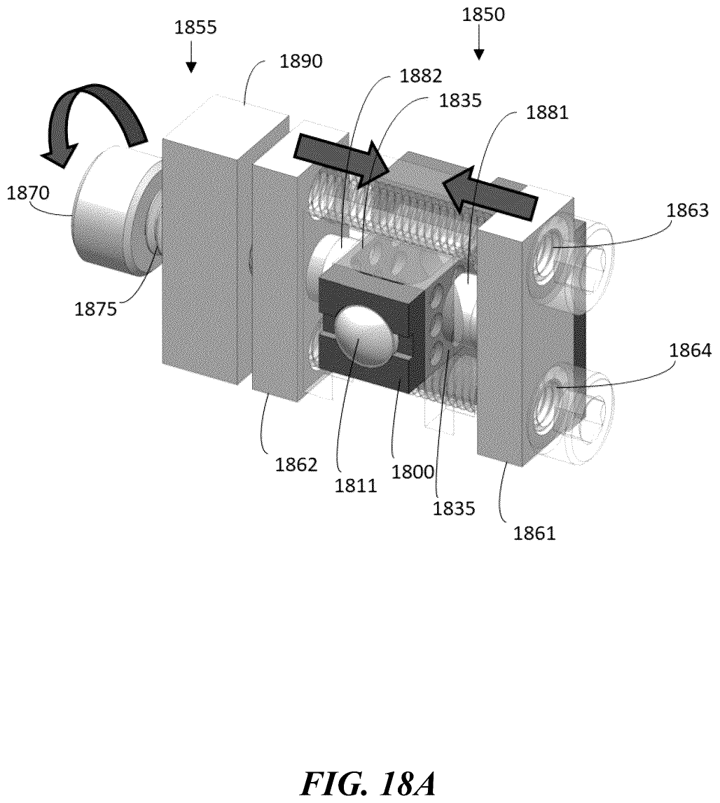

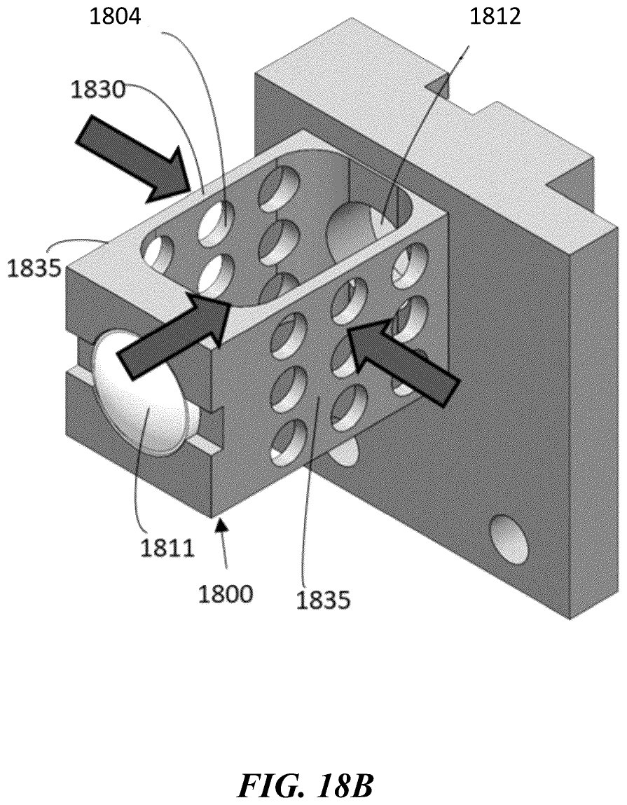

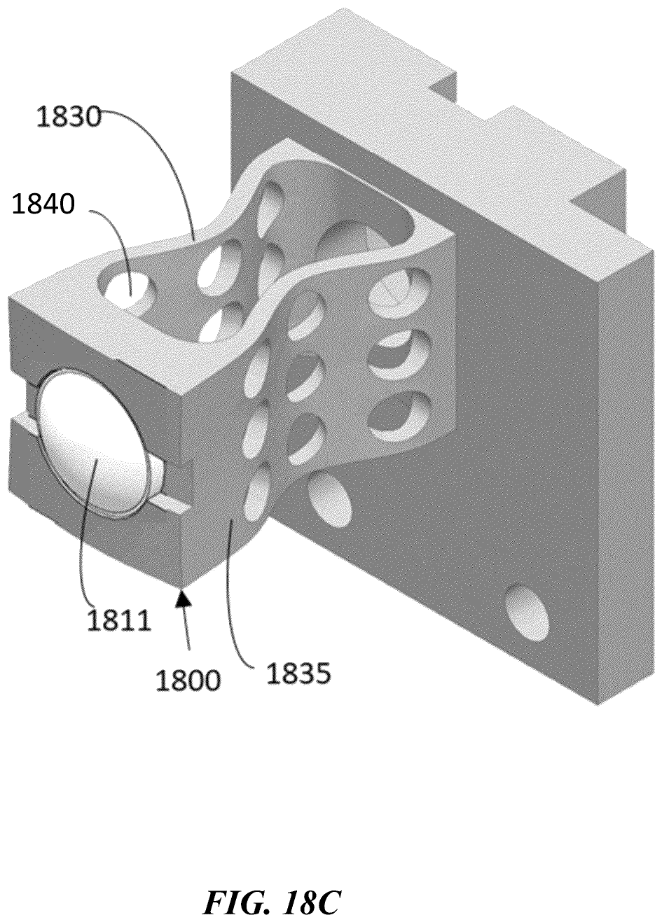

FIGS. 18A-18C illustrate a beam de-expander (e.g., the beam de-expander unit) 1800 configured to be deformed to change the length between a first lens 1811 and a second lens 1812, as similarly described with reference to FIGS. 17A-17D. The lenses 1811, 1812 may be mounted on opposite sides of a chamber of the beam de-expander 1830. A collected (e.g., returned) beam of light may pass through the lenses 1811, 1812 and the chamber of the beam de-expander 1830. The collected light beam may include a collimated beam before entering the beam de-expander 1800. The collected light beam may comprise a collimated beam after exiting the beam de-expander The chamber of the beam de-expander 1830 may comprise a sidewall 1835. The chamber of the beam de-expander 1830 may comprise a single wall, more than one sidewall, more than two sidewalls, more than three side walls, more than four sidewalls, more than five sidewalls, more than six sidewalls, more than seven sidewalls, more than eight sidewalls, more than nine sidewalls, or more than ten sidewalls. The sidewall 1835 may comprise cutouts or openings 1840 that may permit weakening or deformation of the sidewall 1835. The weakening or deformation of the sidewall 1835 may help reduce the distance between the lenses 1811, 1812 (e.g., reduce the length of the chamber of the beam de-expander 1830 between the first lens 1811 and the second lens 1812). The sidewall 1835 may comprise, for example, a material that maintains its shape when deformed. The material of the sidewall 1835 may comprise a metal, such as aluminum, stainless steel, or any other metal or material that may maintain its shape when deformed.

Referring to FIG. 18A, the beam de-expander (e.g., the beam de-expander unit) 1800 may comprise a tuning structure. The tuning structure may or may not be removable from the beam de-expander 1800. The tuning structure may comprise a press 1850, which may comprise an actuator 1855. The press 1850 and the actuator 1855 may operate to tune the beam de-expander 1800. The press 1850 may comprise a first jaw 1861 and a second jaw 1862. The first jaw 1861 and the second jaw 1862 may comprise an orientation that comprises opposing parallel jaws. The first jaw 1861 and the second jaw 1862 may be movable. The first jaw 1861 and the second jaw 1862 may be fixed. Alternatively, and in addition, the first jaw 1861 may be fixed and the second jaw 1862 may be movable. Alternatively, and in addition, the first jaw 1861 may be movable and the second jaw 1862 may be fixed. The first jaw 1861 and the second jaw 1862 may face each other in a perpendicular orientation with respect to an axis between the two lenses of the chamber of the beam de-expander (e.g., the axis may correspond to the path of a beam passing through the beam de-expander (e.g., the beam de-expander unit)). 1800. The first jaw 1861 may comprise a first die element 1881 and the second jaw 1862 may comprise a second die element 1882. The die elements 1881, 1882 may be fixed to the inner surfaces of the jaws 1861, 1862, respectively. The jaws 1861, 1862 may be positioned on each side respectively, of the beam de-expander 1800 with the die elements 1881, 1882 contacting the sidewalls 1835 of the chamber of the beam de-expander 1830. The jaws 1861, 1862 may be moveably coupled by slidable alignment screws 1863, 1864 (as shown in FIG. 18A) that may align the jaws 1861, 1862 when the jaws 1861, 1862 move towards and away from each other. The jaws 1861, 1862 may move towards and away from each other with a tuning structure that may comprise an actuator 1855. Alternatively, or in addition, the tuning structure may comprise a knob, a lever, one or more screws, one or more jaws, a press, one or more block elements, die elements, or combinations thereof. In some cases throughout, block elements may refer to elements of a die press (e.g., die elements). The die elements may contact the walls of the chamber of the beam de-expander. In some embodiments, the actuator 1855 may comprise a knob 1870 (or a lever) and an additional screw 1875 that may drive a press block 1890 towards the second jaw 1862, which may drive the second jaw 1862 to move inwardly toward the first jaw 1861. In some embodiments, the actuator 1855 may comprise the knob 1870 (or the lever) and the additional screw 1875, and may drive the press block 1890 away from the second jaw 1862, which may cause the second jaw 1862 to move outwardly away from the first jaw 1861. When the press block 1890 is actuated by the actuator 1855, the gap between the jaws 1861, 1862 may be reduced or increased. When the gap between the jaws 1861, 1862 is reduced, a first block element 1883 and a second block element 1884 (as shown in FIG. 18A) may symmetrically compress and deform the sidewalls 1835 of the chamber of the beam de-expander 1830 inwardly.

The distance between the first lens 1811 and the second lens 1812 may be initially set (e.g., during manufacturing) at slightly greater than a desired distance. For example, a desired distance may be the sum of the focal lengths F1, F2, respectively of the lenses 1811, 1812. In other embodiments, the initial distance between the first lens 1811 and the second lens 1812 may be set slightly less or the same as than a distance that comprises the sum of the focal lengths of the lenses 1811, 1812. The tuning structure (e.g., tuning unit, tuning structure unit, tuning device, tuning structure device, tuning mechanism) may be used to tune the beam de-expander (e.g., beam de-expander unit) 1800. To turn the beam de-expander 1800, the tuning structure may be used to determine a beam diameter (e.g., a smaller beam diameter may represent a greater amplified rejection of off-axis light). According to some embodiments a beam profiler may be used to determine the smaller beam diameter. The beam diameter may be monitored as the block press 1890 is actuated by the actuator 1855. After the beam de-expander 1800 is tuned, the elements of the tuning structure (e.g., the press 1850) may be removed.

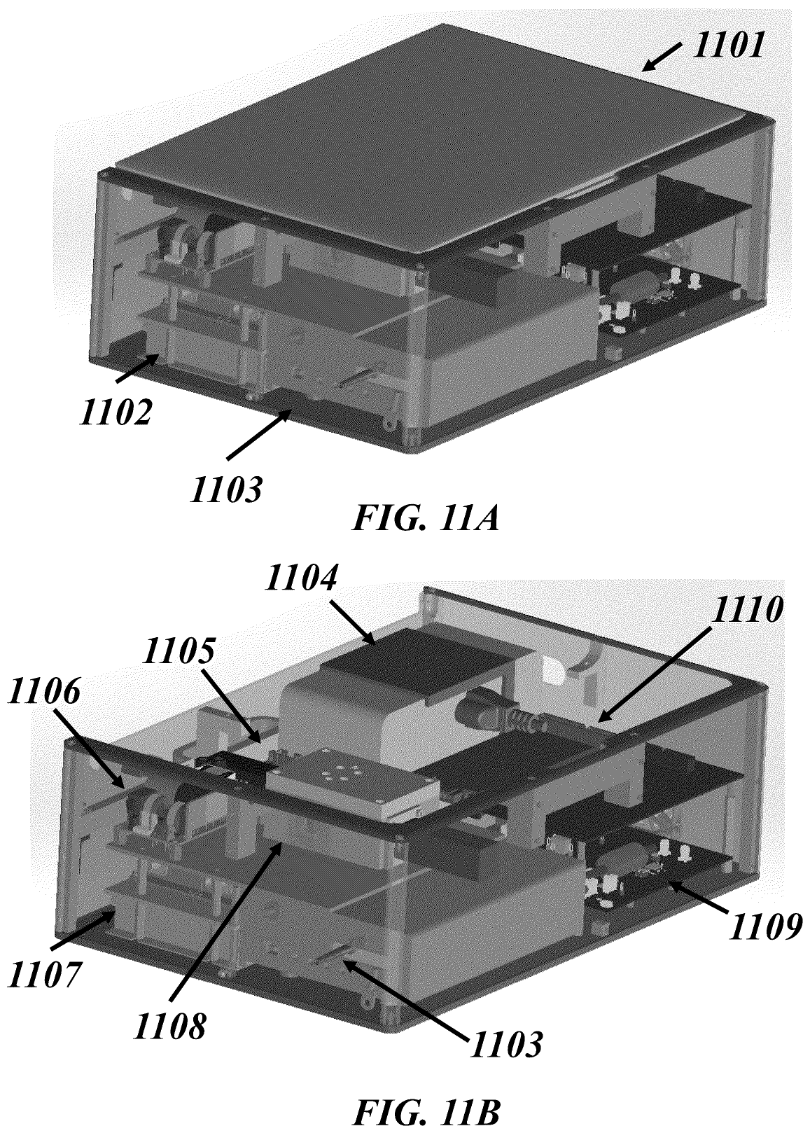

Referring to FIGS. 6 and 7, another subset of the one or more signals may be transmitted through dichroic mirror 290 into a collection arrangement 293, and may be detected by one or more photodetectors as described herein, for example of detector block 1108 of FIG. 11B. The subset of the one or more signals may comprise multi-photon signals for example, that can include SHG or two-photon autofluorescence and/or two-photon fluorescence signals. The collection arrangement 293 may include optical elements (e.g., lenses or mirrors). The collection arrangement 293 may direct the collected light through a light guide to one or more photosensors. The light guide 294 may be a liquid light guide, a multimode fiber, or a bundle of fibers.

The optical device can also have other sensors in addition to the power sensor. The information from the sensors can be used or recorded with the depth profile to provide additional enhanced information with respect to the probe and/or the subject. For example, other sensors within the optical device can comprise position sensors, GPS sensors, temperature sensors, camera or video sensors, dermatoscopes, accelerometers, contact sensors, and humidity sensors.

Referring to FIGS. 1A-3B and 5-7, disclosed herein are apparatuses that may be used among other things, for imaging an image subject. Referring to FIGS. 1A-3B and 5-7, disclosed herein are apparatuses that may be used among other things, for confocal imaging.

Referring to FIGS. 1A-3B and 5-7, disclosed herein are apparatuses that may be used among other things, for generating depth profiles of tissue. In an aspect, an apparatus for generating a depth profile of a tissue of a subject may comprise an optical device that transmits an excitation light beam from form a light source towards a surface of the tissues, which excitation light beam, upon contacting the tissue, generate signals indicative of an intrinsic property of one or more focusing units in the optical device simultaneously adjust a depth and position of the excitation light beam along a scan path, scan pattern or in one or more slant directions, on or more sensors configured to detect at least a subset of the signals generated upon contacting the tissue with the excitation light beam; and one or more computer processors operatively couple to the one or more sensors, wherein the one or more computer processors are individually or collectively programmed to process the at least one subset of the signals detected by the one or more sensors to generate a depth profile of tissue.

The at least a subset of signals may comprise polarized light. The optical device may comprise one or more polarization selective optics (e.g., polarization filters, polarization beam splitters, etc.). The one or more polarization selective optics may select for a particular polarization of RCM signal, such that the RCM signal that is detected is of a particular polarization from a particular portion of the tissue. For example, polarization selective optics can be used to selectively image or amplify different features in tissue.

The at least a subset of signals may comprise unpolarized light. The optical device may be configured to reject up to all out of focus light. By rejecting out of focus light, a low noise image may be generated from RCM signals.

Multiple refractive lenses, such as relay lenses, collimating lenses, and field lenses, may be used to focus the ultrafast pulses of light from a light source to a small spot within the tissue. The small spot of focused light can, upon contacting the tissue, generate endogenous tissue signals, such as second harmonic generation, 2-photon autofluorescence, third harmonic generation, coherent anti-stokes Raman spectroscopy, confocal microscopy signals, or other nonlinear multiphoton generated signals. The probe may also transfer the scanning pattern generated by optical elements such as mirrors and translating lenses to a movement of the focal spot within the tissue to scan the focus through the structures and generate a point-by-point image of the tissue. The probe may comprise multiple lenses to minimize aberrations, optimize the linear mapping of the focal scanning, and maximize resolution and field of view.

The one or more focusing units in the optical device may comprise, but are not limited to, movable lens, an actuator coupled to an optical element (e.g., an afocal lens), MEMS mirror, relay lenses, dichroic mirror, a fold mirror, a beam splitter, a reflection chamber, or any combination thereof as described in detail herein with reference to FIGS. 1A-9. An alignment element to direct confocal microscopy signals into the reflection chamber may comprise but is not limited to, an angular adjustment element, and/or a moveable mirror. The signals indicative of an intrinsic property of the tissue may be signals as described elsewhere herein, such as, for example, confocal microscopy signals, second harmonic generation signals, multi photon fluorescence signals, other generated signals, or any combination thereof.

Apparatuses consistent with the methods herein may comprise any element of the subject methods including, but not limited to, an optical device; one or more light sources such as an ultrashort pulse laser; one or more mobile or tunable lenses; one or more optical filters; one or more photodetectors; one or more computer processors; one or more marking tools; and combinations thereof.

The photodetector may comprise, but are not limited to, a photomultiplier tube (PMT), a photodiode, an avalanche photodiode (APD), a charge-coupled device (CCD) detector, a charge-injection device (CID) detector, a complementary-metal-oxide-semiconductor detector (CMOS) detector, a multi-pixel photon counter (MPPC), a silicon photomultiplier (SiPM), light dependent resistors (LDR), a hybrid PMT/avalanche photodiode sensor, and/or other detectors or sensors. The system may comprise one or more photodetectors of one or more types, and each sensor may be used to detect the same or different signals. For example, a system can use both a photodiode and a CCD detector, where the photodiode detects SHG and multi photon fluorescence and the CCD detects reflectance confocal microscopy signals. The photodetector may be operated to provide a framerate, or number of images obtained per second, of at least about 0.5, 1, 2, 3, 4, 5, 6, 7, 8, 9, 10, 15, 20, 24, or more. The photodetector may be operated to provide a framerate of at most about 60, 50, 40, 30, 24, 20, 15, 10, 9, 8, 7, 6, 5, 4, 3, 2, 1, 0.5, or less.

The optical device may comprise a photomultiplier tube (PMT) that collects the signals. The PMT may comprise electrical interlocks and/or shutters. The electrical interlocks and/or shutters can protect the PMT when the photomultiplier compartment is exposed to ambient light by activating when contact between the surface of the epithelial tissue and the optical prove has been disrupted. By using activatable interlocks and/or shutters, signals can be collected in the presence of ambient light, thereby allowing a user to generate one or more real-time, pre-surgical depth profiles at the bedside of the patient. The optical device may comprise other photodetectors as well.

The light source providing ultrashort pulses of light can be a wavelength-tunable, ultrashort-pulsed Ti:Sapphire laser. A Ti:Sapphire laser can be a mode-locked oscillator, a chirped-pulse amplifier, or a tunable continuous wave laser. A mode-locked oscillator can generate ultrashort pulses with a duration between about a few picoseconds and about 10 femtoseconds, and in cases about 5 femtoseconds. The pulse repetition frequency can be about 70 to 90 megahertz (MHz). The term ‘chirped-pulse’ generally refers to a special construction that can prevent the pulse from damaging the components in the laser. In a ‘chirped-pulse’ laser, the pulse can be stretched in time so that the energy is not all located at the same point in time and space, preventing damage to the optics in the amplifier. The pulse can then be optically amplified and recompressed in time to form a short, localized pulse.

The mobile lens or moveable lens of an apparatus can be translated to yield the plurality of different scan patterns or scan paths. The mobile lens may be coupled to an actuator that translates the lens. The actuator may be controlled by a programmed computer processor. The actuator can be a linear actuator, such as a mechanical actuator, a hydraulic actuator, a pneumatic actuator, a piezoelectric actuator, an electro-mechanical actuator, a linear motor, a linear electric actuator, a voice coil, or combinations thereof. Mechanical actuators can operate by converting rotary motion into linear motion, for example by a screw mechanism, a wheel and axle mechanism, and a cam mechanism. A hydraulic actuator can involve a hollow cylinder comprising a piston and an incompressible liquid. A pneumatic actuator may be similar to a hydraulic actuator but involves a compressed gas instead of a liquid. A piezoelectric actuator can comprise a material which can expand under the application of voltage. As a result, piezoelectric actuators can achieve extremely fine positioning resolution, but may also have a very short range of motion. In some cases, piezoelectric materials can exhibit hysteresis which may make it difficult to control their expansion in a repeatable manner. Electro-mechanical actuators may be similar to mechanical actuators. However, the control knob or handle of the mechanical actuator may be replaced with an electric motor.

Tunable lenses can refer to optical elements whose optical characteristics, such as focal length and/or location of the optical axis, can be adjusted during use, for example by electronic control. Electrically-tunable lenses may contain a thin layer of a suitable electro-optical material (e.g., a material whose local effective index of refraction, or refractive index, changes as a function of the voltage applied across the material). An electrode or array of electrodes can be used to apply voltages to locally adjust the refractive index to the value. The electro-optical material may comprise liquid crystals. Voltage can be applied to modulate the axis of birefringence and the effective refractive index of an electro-optical material comprising liquid crystals. In some cases, polymer gels can be used. A tunable lens may comprise an electrode array that defines a grid of pixels in the liquid crystal, similar to pixel grids used in liquid-crystal displays. The refractive indices of the individual pixels may be electrically controlled to give a phase modulation profile. The phase modulation profile may refer to the distribution of the local phase shifts that are applied to light passing through the layer as the result of the locally-variable effective refractive index over the area of the electro-optical layer of the tunable lens.

In some cases, an electrically or electro-mechanically tunable lens that is in electrical or electro-mechanical communication with the optical device may be used to yield the plurality of different scan patterns or scan paths. Modulating a curvature of the electrically or electro-mechanically tunable lens can yield a plurality of different scan patterns or scan paths with respect to the epithelial tissue. The curvature of the tunable lens may be modulated by applying current. The apparatus may also comprise a programmed computer processor to control the application of current.

An apparatus for identifying a disease in an epithelial tissue of a subject may comprise an optical device. The optical device may transmit an excitation light beam from a light source towards a surface of the epithelial tissue. The excitation light beam, upon contacting the epithelial tissue, can then generate signals that relate to an intrinsic property of the epithelial tissue. The light source may comprise an ultra-fast pulse laser, such as a Ti:Sapphire laser. The ultra-fast pulse laser may generate pulse durations less than 500 femtoseconds, 400 femtoseconds, 300 femtoseconds, 200 femtoseconds, 100 femtoseconds, or less. The pulse repetition frequency of the ultrashort light pulses can be at least 10 MHz, 20 MHz, 30 MHz, 40 MHz, 50 MHz, 60 MHz, 70 MHz, 80 MHz, 90 MHz, 100 MHz, or greater.

The tissue may be epithelial tissue. The depth profile may permit identification of the disease in or state of the epithelial tissue of the subject. The disease in the tissue of the subject is disclosed elsewhere herein.

The scanning path or pattern may be in one or more slant directions and on one or more slanted planes. A slanted plane may be positioned along a direction that is angled with respect to an optical axis of the optical device. The angle between a slanted plane and the optical axis may be at most 45°. The angle between a slanted plane and the optical axis may be at least about 5°, 10°, 15°, 20°, 25°, 30°, 35°, 40°, 45°, 55°, 60°, 65°, 70°, 75°, 80°, 85°, or greater. In other cases, the angle between a slanted plane and the optical axis may be at most about 85°, 80°, 75°, 70°, 65°, 60°, 55°, 50°, 45°, 35°, 30°, 25°, 20°, 15°, 10°, 5°, or less.

The optical device may further comprise one or more optical filters, which one or more optical filters may be configured to collect a subset of the signals. Optical filters, as described elsewhere herein, can be used to collect one or more specific subsets of signals that relate to one or more intrinsic properties of the epithelial tissue. The optical filters may be a beam splitter, a polarizing beam splitter, a notch filter, a dichroic filter, a long pass filter, a short pass filter, a bandpass filter, or a response flattening filter. The optical filters may be one or more optical filters. These optical filters can be coated glass or plastic elements which can selectively transmit certain wavelengths of light, such as autofluorescent wavelengths, and/or light with other specific attributes, such as polarized light. The optical filters can collect at least one signal selected from the group consisting of second harmonic generation (SHG) signal, third harmonic generation (THG) signal, polarized light signal, reflectance confocal microscopy (RCM) signal, and autofluorescence signal. The subset of the signals may include at least one of second harmonic generation (SHG) signals, third harmonic generation (THG) signals, and autofluorescence signals.

The light source may comprise an ultra-fast pulse laser with pulse durations less than about 200 femtoseconds. An ultra-fast pulse laser may produce pulses of light with pulse durations at most 500 femtoseconds, 450 femtoseconds, 400 femtoseconds, 350 femtoseconds, 300 femtoseconds, 250 femtoseconds, 200 femtoseconds, 150 femtoseconds, 100 femtoseconds, or shorter. In some cases, the pulse duration is about 150 femtoseconds. In some cases, an ultra-fast pulse laser may produce pulses of light with pulse durations at least 100 femtoseconds, 150 femtoseconds, 200 femtoseconds, 250 femtoseconds, 300 femtoseconds, 350 femtoseconds, 400 femtoseconds, 450 femtoseconds, 500 femtoseconds, or shorter. The pulse repetition frequency of an ultra-fast pulse laser can be at least 10 MHz, 20 MHz, 30 MHz, 40 MHz, 50 MHz, 60 MHz, 70 MHz, 80 MHz, 90 MHz, 100 MHz, or greater. In some cases, the pulse repetition frequency of an ultra-fast pulse laser can be at most 100 MHz, 90 MHz, 80 MHz, 70 MHz, 60 MHz, 50 MHz, 40 MHz, 30 MHz, 20 MHz, 10 MHz, or less. In some cases, the pulse repetition frequency is about 80 MHz.

During use, the probe of the optical device may be in contact with the surface of the tissue. Alternatively, or in addition to, the probe may be configured to penetrate the tissue. The probe may include one or more objectives. An objective may contact the surface of the tissue. The objective(s) may be configured to collimate the light. The contact may be direct or indirect contact. If the contact is a direct contact, performing the contact may comprise placing the optical device next to the tissue of the subject without an intervening layer. If the contact is an indirect contact, performing the contact may comprise placing the probe of the optical device next to the tissue of the subject with one or more intervening layers. The one or more intervening layers may comprise, but are not limited to, clothes, medical gauzes, bandages, and so forth. The contact may be monitored such that when contact between the surface of the epithelial tissue and the optical device is disrupted, a shutter positioned in front of the detector (e.g., relative to the path of light) can be activated and block incoming light. In some cases, the photodetector comprises electrical interlocks and/or shutters. The electrical interlocks and/or shutters can protect the photodetector when the photomultiplier compartment is exposed to ambient light by activating when contact between the surface of the epithelial tissue and the optical prove has been disrupted. By using activatable interlocks and/or shutters, signals can be collected in the presence of ambient light, thereby allowing a user to generate one or more real-time, pre-surgical depth profiles at the bedside of the patient.

The apparatus may comprise a sensor that detects a displacement between the probe and the surface of the tissue. This sensor can protect the photodetector, for example a photodetector, from ambient light by activating a shutter or temporarily deactivating the photodetector to prevent ambient light from reaching and damaging the photodetector, if the ambient light exceeds the detection capacity of the photodetector.

The optical device may comprise a power meter. The power meter may be optically coupled to the light source. The power meter may be used to correct for fluctuations of the power of the light source. The power meter may be used to control the power of the light source. For example, an integrated power meter can allow for setting a power of the light source depending on how much power is used for a particular imaging session. The power meter may ensure a consistent illumination over a period of time, such that images obtained throughout the period of time have similar illumination conditions. The power meter may provide information regarding the power of the illumination light to the system processing that can be recorded with the depth profile. The power information can be included in the machine learning described elsewhere herein. The power meter may be, for example, a photodiode, a pyroelectric power meter, or a thermal power meter. The power meter may be a plurality of power meters.

The apparatus may further comprise a marking tool for outlining a boundary that is indicative of a location of the disease in the epithelial tissue of the subject. The marking tool can be a pen or other writing instrument comprising skin marking ink that is FDA approved, such as Genetian Violet Ink; prep resistant ink that can be used with aggressive skin prep such as for example CHG/isopropyl alcohol treatment; waterproof permanent ink; or ink that is easily removable such as with an alcohol. A pen can have a fine tip, an ultra-fine tip, or a broad tip. The marking tool can be a sterile pen. As an alternative, the marking tool may be a non-sterile pen.

The apparatus may be a portable apparatus. The portable apparatus may be powered by a battery. The portable apparatus may comprise wheels. The portable apparatus may be contained within a housing. The housing can have a footprint of greater than or equal to about 0.1 ft², 0.2 ft², 0.3 ft², 0.4 ft², 0.5 ft², 1 ft², or more. As an alternative, the housing can have a footprint that is less than or equal to about 1 ft², 0.5 ft², 0.4 ft², 0.3 ft², 0.2 ft², or 0.1 ft². The portable apparatus may comprise a filtered light source that emits light within a range of wavelengths not detectable by the optical device.

The portable apparatus may be at most 50 lbs, 45 lbs, 40 lbs, 35 lbs, 30 lbs, 25 lbs, 20 lbs, 15 lbs, 10 lbs, 5 lbs or less. In some cases, the portable apparatus may be at least 5 lbs, 10 lbs, 15 lbs, 20 lbs, 25 lbs, 30 lbs, 35 lbs, 40 lbs, 45 lbs, 50 lbs, 55 lbs or more.

The optical device may comprise a handheld housing configured to interface with a hand of a user. An optical device that can be translated may comprise a handheld and portable housing. This can allow a surgeon, physician, nurse, or other healthcare practitioner to examine in real-time the location of the disease, for example a cancer in skin tissue, at the bedside of a patient. The portable apparatus can have a footprint of greater than or equal to about 0.1 ft², 0.2 ft², 0.3 ft², 0.4 ft², 0.5 ft², or 1 ft². As an alternative, the portable apparatus can have a footprint that is less than or equal to about 1 ft², 0.5 ft², 0.4 ft², 0.3 ft², 0.2 ft², or 0.1 ft².

The probe may have a tip diameter that is less than about 10 millimeters (mm), 8 mm, 6 mm, 4 mm, or 2 mm. The handheld device may have a mechanism to allow for the disposable probe to be easily connected and disconnected. The mechanism may have an aligning function to enable precise optical alignment between the probe and the handheld device. The handheld device may be shaped like an otoscope or a dermatoscope with a gun-like form factor. The handheld device may have a weight of at most about 8 pounds (lbs), 4 lbs, 2 lbs, 1 lbs, 0.5 lbs, or 0.25 lbs. A screen may be incorporated into the handheld device to give point-of-care viewing. The screen may be detachable and able to change orientation. The handheld device may be attached to a portable system which may include a rolling cart or a briefcase-type configuration. The portable device may comprise a screen. The portable device may comprise a laptop computing device, a tablet computing device, a computing device coupled to an external screen (e.g., a desktop computer with a monitor), or a combination thereof. The portable system may include the laser, electronics, light sensors, and power system. The laser may provide light at an optimal frequency for delivery. The handheld device may include a second harmonic frequency doubler to convert the light from a frequency useful for delivery (e.g., 1,560 nm) to one useful for imaging tissue (e.g., 780 nm). For example, the delivery frequency may be at least about 800 nm, 900 nm, 1,000 nm, 1,100 nm, 1,200 nm, 1,300 nm, 1,400 nm, 1,500 nm, 1,600 nm, 1,700 nm, 1,800 nm, 1,900 nm, or more and the imaging frequency may be at least about 400 nm, 450 nm, 500 nm, 550 nm, 600 nm, 650 nm, 700 nm, 750 nm, 800 nm, 850 nm, 900 nm, 950 nm or more. The laser may be of low enough power to run the system on battery power. The system may further comprise a charging dock or mini-stand to hold the portable unit during operation. There may be many mini-stands in a single medical office and a singly portable system capable of being transported between rooms.

The housing may further comprise an image sensor. Alternatively, the image sensor may be located outside of the housing. In either case, the image sensor may be configured to locate the optical device housing in space. The image sensor may locate the optical device housing in space by tracking one or more features around the optical device. The image sensor may be a video camera. The one or more features may be features of the tissue (e.g., freckles, birthmarks, etc.) or markers on or in the tissue placed by practitioners. The one or more features may be features of the space wherein the optical device is used (e.g., furniture, walls, etc.). For example, the housing can have a number of cameras integrated into it that use a computer algorithm to track the position of the housing by tracking the movement of the furniture of the room the optical device is being used in, and the tracking can be used to help generate a complete 3D image of a section of a tissue. By simultaneously tracking the position of the housing or optical device position while recording images of tissue, a computer can reconstruct the location of the image within the tissue as the housing translates. In this way a larger mosaic region of the tissue can be imaged and digitally reconstructed. Such a region can be a 3D volume, or a 2D mosaic, or an arbitrary surface within the tissue. The image sensor may be configured to detect light in the near infrared. The housing may be configured to project a plurality of points to generate a map for the image sensor to use for tracking. In addition to using an image sensor, one or more position sensors, one or more other guides, or one or more sensors may be used with or by the optical device or housing to locate the probe position with respect to the location of tissue features or tissue characteristics. A processor can identify the optical device position with respect to currently or previously collected data. For example, identified features of the tissue can be used to identify, mark, or notate probe position. Current or previously placed tags or markers can also be used to identify probe position with respect to the tissue. Such tags or markers can include, without limitation, dyes, wires, fluorescent tracers, stickers, inked marks, incisions, sutures, mechanical fiducials, mechanical anchors, or other elements that can be sensed. A guide can be used with an optical device to direct, mechanically reference, and/or track optical probe position. Optical probe position data can be incorporated into image data that is collected to create a depth profile.

The housing may contain optical elements configured to direct the at least a subset of the signals to one or more detectors. The one or more detectors may be optically coupled to the housing via one or more fiber optics. The housing may contain the one or more detectors as well as a light source, thus having an entirely handheld imaging system.

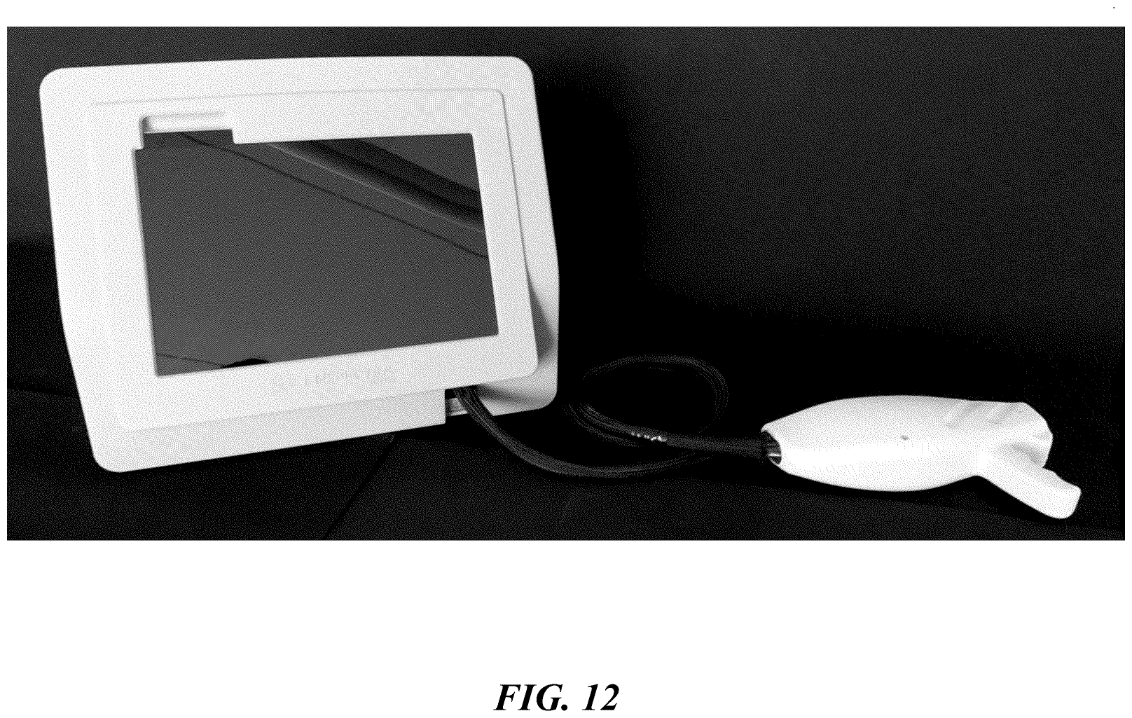

FIGS. 12-14 show an example of an optical device housing coupled to a support system. FIGS. 11A and 11B show the inside of an example support system. A portable computing device 1101 may be placed on top of the support system. The support system may comprise a laser 1103. The support system may comprise a plurality of support electronics, such as, for example, a battery 1104, a controller 1102 for the afocal lens actuator a MEMS mirror driver 1105, a power supply 1106, one or more transimpedance amplifiers 1107, a photodetector block 1108, a plurality of operating electronics 1109, a data acquisition board 1110, other sensors or sensor blocks or any combination thereof.

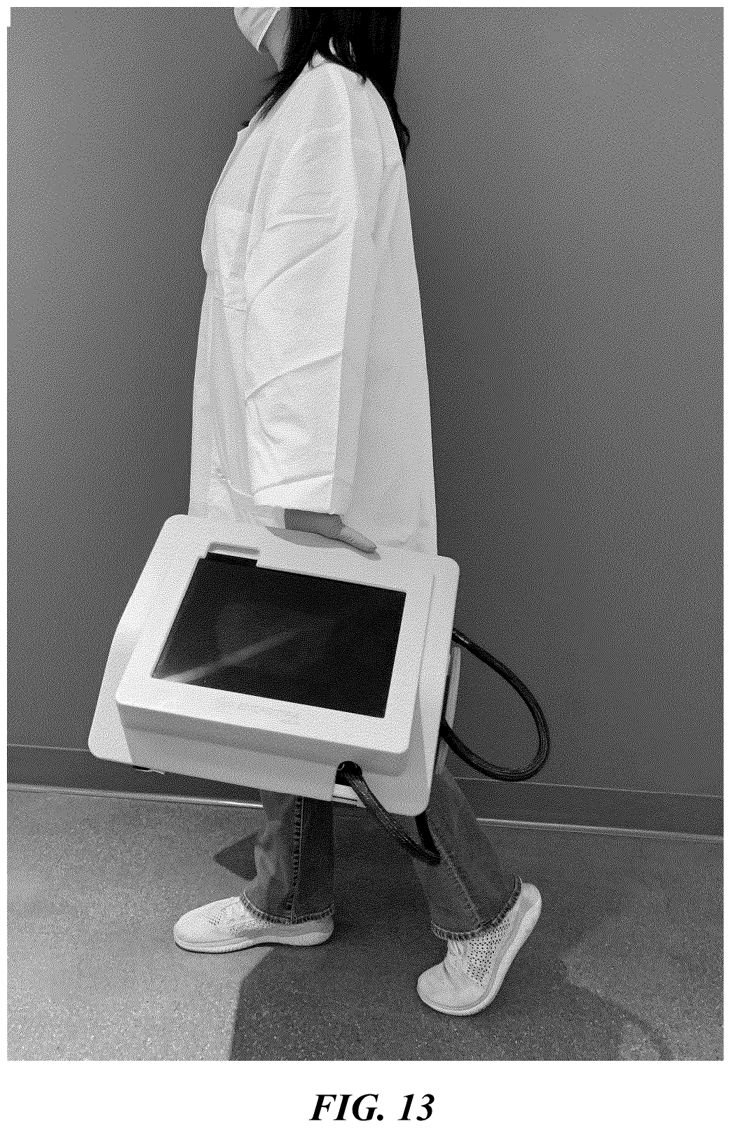

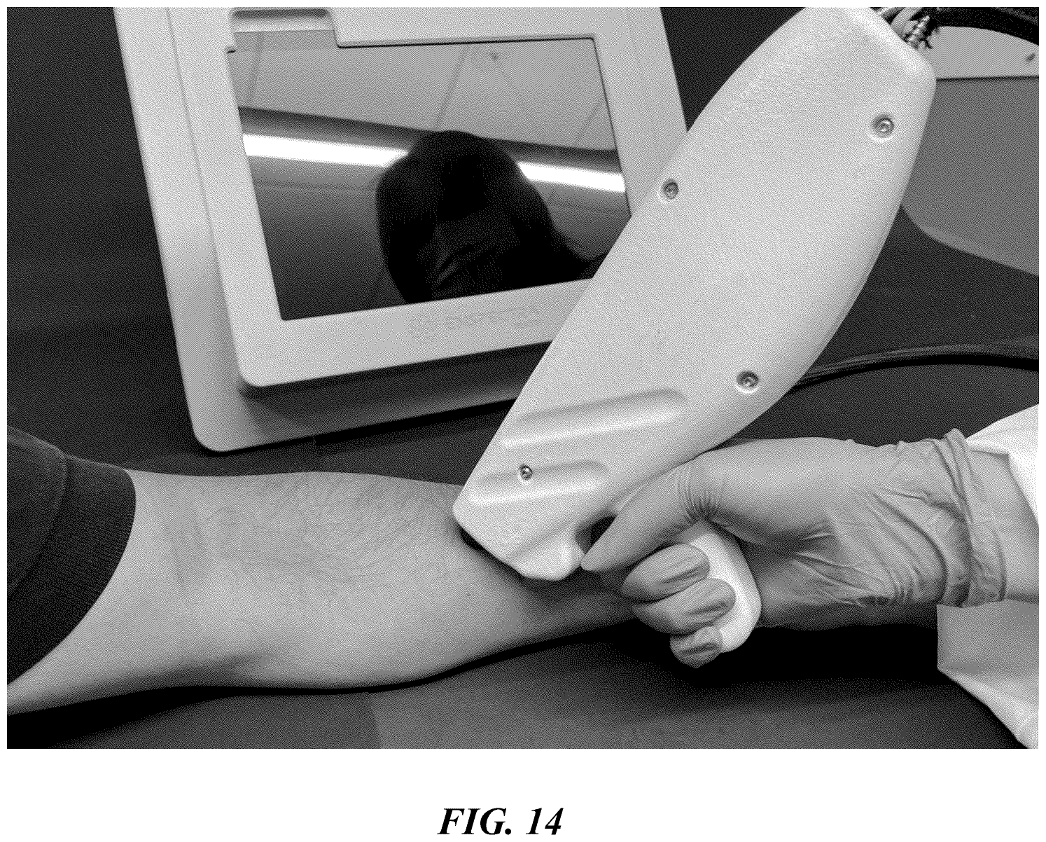

FIG. 13 shows an example of the portability of the example of FIG. 12. FIG. 14 shows an example system in use. Support system may send a plurality of optical pulses to a housing via connecting cable. The plurality of optical pulses may interact with tissue generating a plurality of signals. The plurality of signals may travel along the connecting cable back to the support system. The support system may comprise a portable computer. The portable computer may process the signals to generate and display an image that can be formed from a depth profile and collected signals as described herein. Preparation of a subject for imaging may include the use of alcohol swabs to clean a tissue of a subject for imaging. Additionally, an optical liquid or gel, such as a drop of glycerol or oil may be applied to a tissue of a subject. Imaging may be performed in the absence of hair removal, stains, drugs, or immobilization.

The one or more computer processors may be operatively coupled to the one or more sensors. The one or more sensors may comprise an infrared sensor, optical sensor, microwave sensor, ultrasonic sensor, radio-frequency sensors, magnetic sensor, vibration sensor, acceleration sensor, gyroscopic sensor, tilt sensor, piezoelectric sensor, pressure sensor, strain sensor, flex sensor, electromyographic sensor, electrocardiogramansor, electroencephalographic sensor, thermal sensor, capacitive touch sensor, or resistive touch sensor.

According to some embodiments of the methods and apparatuses herein, an image can be depth profile as described herein and can include additional data as described herein. The depth profile may be an image. The images can also be portions of depth profiles as described herein and can be in the form of tiles or portions of image data. The images can be obtained in vivo. The signals can be collected and images, depth profiles, tiles, or datasets can be created without removing tissue from the body of the subject or fixing the tissue to a slide. The images can extend below a surface of the tissue. The images can have a resolution of at least about 1, 5, 10, 25, 50, 75, 100, 150, 200, 250, 300, 400, 500, 600, 700, 800, 900, 1,000 or more micrometers. The images can have a resolution of at most about 1,000, 900, 800, 700, 600, 500, 400, 300, 250, 200, 150, 100, 75, 50, 25, 10, 5, 1, or fewer micrometers. The images can comprise optical images.

Images obtained with the optical device can be used to detect or identify a tissue characteristic in a subject or to generate a data set for a trained algorithm and generated trained algorithm for classifying images of tissues from a subject. The trained algorithm may be as described, for example, with reference to PCT/US2019/061306, filed Nov. 13, 2019, and PCT/US2020/060302, filed Nov. 12, 2020, each of which is entirely incorporated herein by reference.

The imaging probe may be configured to measure one or more electronic signals. The electronic signal may be or may be indicative of a current, a voltage, a charge, a resistance, a capacitance, a conductivity, an impedance, any combination thereof, or a change thereof. The imaging probe may comprise imaging optics. The imaging probe may be configured to measure one or more optical signals. Examples of imaging probes, including handheld optical devices, are provided elsewhere herein. Signals received by the imaging probe can be used to generate images of tissue regions from which signals were received. The imaging probe may be handheld. The imaging probe may be translated, lifted, or the orientation may be changed. For example, an imaging probe can be placed at an angle on a subject's skin and rotated to view tissue in a different location.

The imaging probe may be operatively coupled to the one or more computer processors. For example, the imaging probe may be plugged into a computer comprising the processors. In another example, the imaging probe may be connected to the one or more computer processors via a network. The imaging probe may be handheld. The imaging probe may be configured to deliver therapy to the tissue as described elsewhere herein.

The signals may be substantially simultaneously (e.g., signals generated within a time period less than or equal to about 30 seconds(s), 20 s, 10 s, 1 s, 0.5 s, 0.4 s, 0.3 s, 0.2 s, 0.1 s, 0.01 s, 0.005 s, 0.001 s, or less; signals generated by the same pulse or beam of light, etc.) generated within a single region of the tissue (e.g., signals generated within less than or equal to about 1, 1E-1, 1E-2, 1E-3, 1E-4, 1E-5, 1E-6, 1E-7, 1E-8, 1E-9, 1E-10, 1E-11, 1E-12, 1E-13 or less cubic centimeters). The signals may be generated by the same pulse or beam of light. The signals may be generated by multiple beams of light synchronized in time and location as described elsewhere herein. Two or more of the signals may be combined to generate a composite image. The signals or subset of signals may be generated within a single region of the tissue using the same or similar scanning pattern or scanning plane. Each signal of a plurality of signals may be independent from the other signals of the plurality of signals. A user can decide which subset(s) of signals to use. For example, when both RCM and SHG signals are collected in a scan, a user can decide whether to use the RCM signals, multiphoton signals, or any combination thereof. Additionally, video tracking of the housing or optical device position as described previously herein can be recorded simultaneously with the generated signals.

The data can be stored in a database. A database can be stored in computer readable format. A computer processor may be configured to access the data stored in the computer readable memory. A computer system may be used to analyze the data to obtain a result. The result may be stored remotely or internally on storage medium and communicated to personnel such as medication professionals. The computer system may be operatively coupled with components for transmitting the result. Components for transmitting can include wired and wireless components. Examples of wired communication components can include a Universal Serial Bus (USB) connection, a coaxial cable connection, an Ethernet cable such as a Cat5 or Cat6 cable, a fiber optic cable, or a telephone line. Examples or wireless communication components can include a Wi-Fi receiver, a component for accessing a mobile data standard such as a 3G or 4G LTE data signal, or a Bluetooth receiver. All these data in the storage medium may be collected and archived to build a data warehouse.

The depth profile can comprise a monochromatic image displaying colors derived from a single base hue. Alternatively, or additionally, the depth profile can comprise a polychromatic image displaying more than one color. In a polychromatic image, color components may correspond to multiple depth profiles using signals or subsets of signals that are synchronized in time and location. Such depth profiles, for example, may be generated using the optical device as described elsewhere herein. Such depth profiles can comprise individual components, images or depth profiles created from a plurality of subsets of gathered and processed generated signals. In some embodiments, the depth profiles correspond to different positions of an optical probe on the tissue. The depth profile may comprise a plurality of layers created from a plurality of subsets of images collected from the same location and time. Each of the plurality of layers may comprise data that identifies different anatomical structures and/or characteristics than those of the other layer(s). In some embodiments, the plurality of depth profiles corresponds to different scan patterns at the time of detecting the signals. In some embodiments, the different scan patterns correspond to a same time and probe position. In some embodiments, at least one scanning pattern of the different scan patterns comprises a slanted scanning pattern. In some embodiments, the slanted scanning pattern forms a slanted plane.

Such depth profiles may comprise a plurality of sub-set depth profiles. In this manner multiple colors can be used to highlight different elements of the tissue such as cells, nuclei, cytoplasm, connective tissues, vasculature, pigment, and tissue layer boundaries. The contrast can be adjusted in real-time to provide and/or enhance structure specific contrast. The contrast can be adjusted by a user (e.g. surgeon, physician, nurse, or other healthcare practitioner) or a programmed computer processor may automatically optimize the contrast in real-time. In a polychromatic image, each color may be used to represent a specific subset of the signals collected, such as second harmonic generation signals, third harmonic generation signals, signals resulting from polarized light, and autofluorescence signals. The colors of a polychromatic depth profile can be customized to reflect the image patterns a surgeon and/or pathologist may see when using standard histopathology. A pathologist may more easily interpret the results of a depth profile when the depth profile is displayed similar to how a traditional histological sample, for example a sample stained with hematoxylin and eosin, may be seen.

The optical device and the one or more computer processors may comprise a same device. The device may be a mobile device. The device may be a plurality of devices that may be operatively coupled to one another. For example, the system can be a handheld optical device optically connected to a laser and detection box, and the box can also contain a computer.

The optical device may be part of a device, and the one or more computer processors may be separate from the device. The one or more computer processors may be part of a computer server. The one or more processors may be part of a distributed computing infrastructure. For example, the system can be a handheld optical device containing all of the optical components that is wirelessly connected to a remote server that processes the data from the optical device.

### Methods for Light Filtering and Imaging

Disclosed herein are methods for generating, among other things, a depth profile of a tissue of a subject. In an aspect, a method for generating a depth profile of an imaging subject (e.g., tissue of a subject) may comprise using an optical device to transmit an excitation light beam from a light source towards a surface of the tissue, which excitation light beam, upon contacting the tissue, generate signals indicative of an intrinsic property of the tissue; using one or more focusing units in the optical device to simultaneously adjust a depth and a position of a focal point of the excitation light beam in a scanning pattern; detecting at least a subset of the signals generated upon contacting the tissue with the excitation light beam; and using one or more computer processors programmed to process the at least the subset of the signals detected to generate the depth profile of the tissue. The scanning pattern can comprise a plurality of focal points. The method described herein for generating a depth profile can alternatively utilize a combination of two or more light beams that are either continuous or pulsed and are collocated at the focal point.

In an aspect, the present disclosure provides a method for filtering light and imaging. The method for filtering light may include providing a light filtering device. The light filtering device may include a chamber, a first reflector, and a second reflector. A distance in the chamber may separate the first reflector and the second reflector. The method may further include directing a beam of light into the chamber to the first reflector, using the first reflector to direct at least a portion of the beam of light from the first reflector to the second reflector in the chamber, and using the second reflector to direct at least another portion of the beam of light from the second reflector to the first reflector in the chamber. The beam of light may repeatedly traverse between the first reflector and the second reflector. A pathlength that the beam of light traverses between the first reflector and the reflector may be at least three times the distance separating the first reflector and the second reflector.

In another aspect, the present disclosure provides a method for filtering light and imaging. The method for imaging an imaging subject may include providing a probe in optical communication with a light filtering device comprising a chamber and the imaging subject. The method may further include using the probe to provide a beam of light to the imaging subject and collect the beam of light from the imaging subject, directing the beam of light from the imaging subject to the chamber of the light filtering device, repeatedly directing the beam of light, directly or indirectly, from a first location to a second location within the chamber of the light filtering device such that a pathlength that the beam of light traverses between the first location and the second location is at least three times a distance separating the first location and the second location, and processing the beam of light to generate and image of the imaging subject.

The methods and systems disclosed herein may be used to form a depth profile of a sample of tissue by utilize scanning patterns that move an imaging beam focal point through the sample in directions that are slanted or angled with respect to the optical axis, in order to improve the resolution of the optical system imaging the samples (e.g., in vivo biologic tissues). The scanner can move its focal points in a line or lines and/or within a plane or planes that are slanted with respect to the optical axis in order to create a depth profile of tissue. The depth profile can provide a projected vertical cross section image generally or approximately representative of a cross section of the tissue that can be used to identify a possible disease state of the tissue. The methods and systems may provide a projected vertical cross section image of an in vivo sample of intact biological tissue formed from depth profile image components (e.g. scanned pattern of focal points). The methods and systems disclosed herein may also produce an image of tissue cross section that is viewed as a tissue slice but may represent different X-Y positions.

According to some embodiments the methods and systems disclosed herein may utilize a slanted plane or planes (or slanted focal plane or planes) formed by a scanning pattern of focal points within the slanted plane or planes. A system that can simultaneously control the X, Y, and Z positions of a focused spot may move the focus through a trajectory in the tissue. The trajectory can be predetermined, modifiable or arbitrary. A substantial increase in resolution may occur when scanning at an angle to the vertical Z axis (e.g., optical axis). The effect may arise, for example, because the intersection between a slanted plane and the point spread function (PSF) is much smaller than the PSF projection in the XZ or YZ plane. Thus, the effective PSF for a focused beam moved along a slanted line or in a slated plane may be smaller as the slant angle increases, approaching the lateral PSF resolution at an angle of 90° (at which point a scan direction line or scan plane can lie within the XY (lateral) plane). Slanted scanning or imaging as described herein, may be used with any type of return signal. Non-limiting examples of return signals can include generated signals described elsewhere herein.

A depth profile through tissue can be scanned at an angle (e.g., more than 0° and less than) 90° with respect to the optical axis, to ensure a portion of the scan trajectory is moving the focus in the Z direction. In some examples, modest slant angles may produce a substantial improvement in resolution. The effective PSF size can be approximated as PSFₗₐₜₑᵣₐₗ/sin(θ) for modest angles relative to the Z axis, where θ is the angle between the z axis and the imaging axis. Additional detail may be found in FIG. 3. Thus, at a scan angle of 45°, the resolution along the depth axis of the slanted plane may be a factor of 1.414 larger than the lateral resolution. With submicron lateral resolution, near or sub-micron slant resolution may be achieved depending on the scan angle. The process may produce cross sectional resolution that is achievable with much higher numerical aperture (NA) optical systems. By operating at a more modest NA, the optics may be more robust to off axis aberrations and can scan larger fields of view and/or greater depths. Additionally, operating at a more modest NA may enable a smaller footprint for an imaging device while maintaining a high resolution.

When the projected cross section image is constructed, the depth profile information derived from the generated signals resulting from the slant scanning, may be projected onto the XZ or YZ plane to create an image plane. In this situation, there may be no distortion in depths of structures relative to the surface of the tissue. This projected cross section image, in some representative embodiments, can comprise data corresponding to a plane optically sliced at one or more angles to the vertical. A projected cross section image can have vastly improved resolution while still representing the depths of imaged structures or tissue.

The depth profile can be generated by scanning a focal point in a in a scanning pattern that includes one or more slanted directions. The scanning may or may not be in a single plane. The scanning may be in a slanted plane or planes. The scanning may be in a complex shape, such as a spiral, or in a predetermined, variable, or random array of points. A scanning pattern, a scanning plane, a slanted plane, and/or a focal plane may be a different plane from a visual or image cross section that can be created from processed generated signals. The image cross section can be created from processed generated signals resulting from moving imaging focal points across a perpendicular plane, a slanted plane, a non-plane pattern, a shape (e.g., a spiral, a wave, etc.), or a random or pseudorandom assortment of focal points.

The depth profile can be generated in real-time. For example, the depth profile may be generated while the optical device transmits one or more excitation light beams from the light source towards the surface of the tissue. The depth profile may be generated at a frame rate of at least 1 frame per second (FPS), 2 FPS, 3 FPS, 4 FPS, 5 FPS, 10 FPS, or greater. In some cases, the depth profile may be generated at a frame rate of at most 10 FPS, 5 FPS, 4 FPS, 3 FPS, 2 FPS, or less. Frame rate may refer to the rate at which an imaging device displays consecutive images called frames. An image frame of the depth profile can provide a cross-sectional image of the tissue.

The image frame, or the area of an image, may be a quadrilateral with any suitable dimensions. An image frame may be rectangular, in some cases with equal sides (e.g., square), for example, depicting a 200 μm by 200 μm cross-section of the tissue. The image frame may depict a cross-section of the tissue having dimensions of at least about 50 μm by 50 μm, 100 μm by 100 μm, 150 μm by 150 μm, 200 μm by 200 μm, 250 μm by 250 μm, 300 μm by 300 μm, or greater. In some cases, the image frame may depict a cross-section of the tissue having dimensions of at most about 300 μm by 300 μm, 250 μm by 250 μm, 200 μm by 200 μm, 150 μm by 150 μm, 100 μm by 100 μm, 50 μm by 50 μm, or smaller. The image frame may not have equal sides.

The image frame may be at any angle with respect to the optical axis. For example, the image frame may be at an angle that is greater than about 0°, 5°, 10°, 15°, 20°, 25°, 30°, 35°, 40°, 45°, 50°, 60°, 70°, 80°, 90°, or more, with respect to the optical axis. The image frame may be at an angle that is less than or equal to about 90°, 85°, 80°, 75°, 70°, 65°, 60°, 50°, 40°, 30°, 20°, 10° 5°, or less, with respect to the optical axis. In some cases, the angle is between any two of the values described above or elsewhere herein, e.g., between 0° and 50°.

The image frame may be in any design, shape, or size. Examples of shapes or designs include but are not limited to: mathematical shapes (e.g., circular, triangular, square, rectangular, pentagonal, or hexagonal), two-dimensional geometric shapes, multi-dimensional geometric shapes, curves, polygons, polyhedral, polytopes, minimal surfaces, ruled surfaces, non-orientable surfaces, quadrics, pseudospherical surfaces, algebraic surfaces, miscellaneous surfaces, Riemann surfaces, box-drawing characters, Cuisenaire rods, geometric shapes, shapes with metaphorical names, symbols, Unicode geometric shapes, other geometric shapes, or partial shapes or combination of shapes thereof. The image frame may be a projected image cross section image as described elsewhere herein.

The excitation light beam may be ultrashort pulses of light. Ultrashort pulses of light can be emitted from an ultrashort pulse laser (herein also referred to as an “ultrafast pulse laser”). Ultrashort pulses of light can have high peak intensities leading to nonlinear interactions in various materials. Ultrashort pulses of light may refer to light having a full width of half maximum (FWHM) on the order of femtoseconds or picoseconds. In some examples, an ultrashort pulse of light has a FWHM of at least about 1 femtosecond, 10 femtoseconds, 100 femtoseconds, 1 picosecond, 100 picoseconds, or 1000 picoseconds or more. In some instances, an ultrashort pulse of light may be a FWHM of at most about 1000 picoseconds, 100 picoseconds, 1 picosecond, 100 femtoseconds, 10 femtoseconds, 1 femtosecond or less. Ultrashort pulses of light can be characterized by several parameters including pulse duration, pulse repetition rate, and average power. Pulse duration can refer to the FWHM of the optical power versus time. Pulse repetition rate can refer to the frequency of the pulses or the number of pulses per second.

Non-limiting examples of ultrashort pulse laser technologies include titanium (Ti):Sapphire lasers, mode-locked diode-pumped lasers, mode-locked fiber lasers, and mode-locked dye lasers. A Ti:Sapphire laser may be a tunable laser using a crystal of sapphire (Al₂O₃) that is doped with titanium ions as a lasing medium (e.g., the active laser medium which is the source of optical gain within a laser). Lasers, for example diode-pumped laser, fiber lasers, and dye lasers, can be mode-locked by active mode locking or passive mode locking, to obtain ultrashort pulses. A diode-pumped laser may be a solid-state laser in which the gain medium comprises a laser crystal or bulk piece of glass (e.g., ytterbium crystal, ytterbium glass, and chromium-doped laser crystals). Although the pulse durations may not be as short as those possible with Ti:Sapphire lasers, diode-pumped ultrafast lasers can cover wide parameter regions in terms of pulse duration, pulse repetition rate, and average power. Fiber lasers based on glass fibers doped with rare-earth elements such as erbium, ytterbium, neodymium, dysprosium, praseodymium, thulium, or combinations thereof can also be used. In some cases, a dye laser comprising an organic dye, such as rhodamine, fluorescein, coumarin, stilbene, umbelliferone, tetracene, malachite green, or others, as the lasing medium, in some cases as a liquid solution, can be used.

The light source providing ultrashort pulses of light can be a wavelength-tunable, ultrashort-pulsed Ti:Sapphire laser. A Ti:Sapphire laser can be a mode-locked oscillator, a chirped-pulse amplifier, or a tunable continuous wave laser. A mode-locked oscillator can generate ultrashort pulses with a duration between about a few picoseconds and about 10 femtoseconds, and in cases about 5 femtoseconds. The pulse repetition frequency can be about 70 to 90 megahertz (MHz). The term ‘chirped-pulse’ generally refers to a special construction that can prevent the pulse from damaging the components in the laser. In a ‘chirped-pulse’ laser, the pulse can be stretched in time so that the energy is not all located at the same point in time and space, preventing damage to the optics in the amplifier. The pulse can then be optically amplified and recompressed in time to form a short, localized pulse.

Ultrashort pulses of light can be produced by gain switching. In gain switching, the laser gain medium is pumped with, e.g., another laser. Gain switching can be applied to various types of lasers including gas lasers (e.g., transversely excited atmospheric (TEA) carbon dioxide lasers). Adjusting the pulse repetition rate can, in some cases, be more easily accomplished with gain-switched lasers than mode-locked lasers, as gain-switching can be controlled with an electronic driver without changing the laser resonator setup. In some cases, a pulsed laser can be used for optically pumping a gain-switched laser. For example, nitrogen ultraviolet lasers or excimer lasers can be used for pulsed pumping of dye lasers. In some cases, Q-switching can be used to produce ultrafast pulses of light.

A type of signal that can be generated or collected for determining a disease in a tissue may be reflectance confocal microscopy (RCM) signals. RCM can use light that is reflected off an imaging subject, such as a tissue, when a beam of light from an optical device is directed to the imaging subject. RCM signals may be a small fraction of the light that is directed to the sample. The RCM signals may be collected by rejecting out of focus light or collecting the in-focus light from a collimated beam, for example as described herein with reference to FIGS. 1A-7. The interaction of the imaging subject with the beam of light may or may not alter the polarization of the RCM signal. Different components of the sample may alter the polarization of the RCM signals to different degrees. The use of polarization selective optics in an optical path of the RCM signals may allow a user to select RCM signal from a given component of the sample. The system can select, split, or amplify RCM signals that correspond to different anatomical features or characteristics to provide additional tissue data. For example, based on the changes in polarization detected by the system, the system can select or amplify RCM signal components corresponding to melanin deposits by selecting or amplifying the RCM signal that associated with melanin, using the polarization selective optics. Other tissue components including but are not limited to collagen, keratin, elastin can be identified using the polarization selective optics. Non-limiting examples of generated signals that may be detected are described elsewhere herein.

Tissue and cellular structures in the tissue can interact with the excitation light beam in a wavelength dependent manner and generate signals that relate to intrinsic properties of the tissue. The signals generated can be used to evaluate a normal state, an abnormal state, a cancerous state, or other features of the tissue in a subject pertaining to the health, function, treatment, or appearance of the tissue, such as skin tissue, or of the subject (e.g., the health of the subject). The subset of the signals generated and collected can include at least one of second harmonic generation (SHG) signals, third harmonic generation (THG) signals, polarized light signals, and autofluorescence signals. A slanted plane imaging technique may be used with any generated signals as described elsewhere herein.

Higher harmonic generation microscopy (HHGM) (e.g., second harmonic generation and third harmonic generation), based on nonlinear multiphoton excitation, can be used to examine cellular structures in live and fixed tissues. SHG can generally refer to a nonlinear optical process in which photons with about the same frequency interact with a nonlinear material and effectively “combine” to generate new photons with about twice the energy, and therefore about twice the frequency and about half (½) the wavelength of the initial photons. Similarly, THG can generally refer to a nonlinear optical process in which photons with about the same frequency interact with a nonlinear material and effectively “combine” to generate new photons with about three times the energy, and therefore about three times the frequency and about one-third (⅓) the wavelength of the initial photons. Second harmonic generation (SHG) and third harmonic generation (THG) of ordered endogenous molecules, such as but not limited to collagen, microtubules, and muscle myosin, can be obtained without the use of exogenous labels and provide detailed, real-time optical reconstruction of molecules including fibrillar collagen, myosin, microtubules as well as other cellular information such as membrane potential and cell depolarization. The ordering and organization of proteins and molecules in a tissue, for example collagen type I and II, myosin, and microtubules, can generate, upon interacting with light, signals that can be used to evaluate the cancerous state of a tissue. SHG signals can be used to detect changes such as changes in collagen fibril/fiber structure that may occur in diseases including cancer, fibrosis, and connective tissue disorders. Various biological structures can produce SHG signals. In some cases, the labeling of molecules with exogenous probes and contrast enhancing agents, which can alter the way a biological system functions, may not be used. In some cases, methods herein for identifying a disease in an epithelial tissue of a subject may be performed in the absence of administering a contrast enhancing agent to the subject.

Another type of signal that can be generated and collected for determining a disease in a tissue may be autofluorescence. Autofluorescence can generally refer to light that is naturally emitted by certain biological molecules, such as proteins, small molecules, and/or biological structures. Tissue and cells can comprise various autofluorescent proteins and compounds. Well-defined wavelengths can be absorbed by chromophores, such as endogenous molecules, proteins, water, and adipose that are naturally present in cells and tissue. Non-limiting examples of autofluorescent fluorophores that can be found in tissues include polypeptides and proteins comprising aromatic amino acids such as tryptophan, tyrosine, and phenylalanine which can emit in the UV range and vitamin derivatives which can emit at wavelengths in a range of about 400 nm to 650 nm, including retinol, riboflavin, the nicotinamide ring of NAD(P)H derived from niacin, and the pyridolamine crosslinks found in elastin and some collagens, which are based on pyridoxine (vitamin B6).

The autofluorescence signal may comprise a plurality of autofluorescence signals. One or more filters may be used to separate the plurality of autofluorescence signals into one or more autofluorescence channels. For example, different parts of a tissue can fluoresce at different wavelengths, and wavelength selective filters can be used to direct each fluorescence wavelength to a different detector. One or more monochromators or diffraction gratings may be used to separate the plurality of autofluorescence signals into one or more channels.

An ultra-fast pulse laser may produce pulses of light with pulse durations at most 500 femtoseconds, 450 femtoseconds, 400 femtoseconds, 350 femtoseconds, 300 femtoseconds, 250 femtoseconds, 200 femtoseconds, 150 femtoseconds, 100 femtoseconds, or shorter. In some cases, the pulse duration is about 150 femtoseconds. In some cases, an ultra-fast pulse laser may produce pulses of light with pulse durations at least 100 femtoseconds, 150 femtoseconds, 200 femtoseconds, 250 femtoseconds, 300 femtoseconds, 350 femtoseconds, 400 femtoseconds, 450 femtoseconds, 500 femtoseconds, or shorter. The pulse repetition frequency of an ultra-fast pulse laser can be at least 10 MHz, 20 MHz, 30 MHz, 40 MHz, 50 MHz, 60 MHz, 70 MHz, 80 MHz, 90 MHz, 100 MHz, or greater. In some cases, the pulse repetition frequency of an ultra-fast pulse laser can be at most 100 MHz, 90 MHz, 80 MHz, 70 MHz, 60 MHz, 50 MHz, 40 MHz, 30 MHz, 20 MHz, 10 MHz, or less. In some cases, the pulse repetition frequency is about 80 MHz.

The collected signals can be processed by a programmed computer processor to generate a depth profile. The signals can be transmitted wirelessly to a programmed computer processor. As an alternative, the signals may be transmitted through a wired connection to a programmed computer processor. The signals or a subset of the signals relating to an intrinsic property of the tissue can be used to generate a depth profile with the aid of a programmed computer processor. The collected signals and/or generated depth profile can be stored electronically. In some cases, the signals and/or depth profile are stored until deleted by a user, such as a surgeon, physician, nurse, or other healthcare practitioner. When used for diagnosis and/or treatment, the depth profile may be provided to a user in real-time. A depth profile provided in real-time can be used as a pre-surgical image to identify the boundary of a disease, for example skin cancer. The depth profile can provide a visualization of the various layers of tissue, such as skin tissue, including the epidermis, the dermis, and/or the hypodermis. The depth profile can extend at least below the stratum corneum, the stratum lucidum, the stratum granulosum, the stratum spinosum or the squamous cell layer, and/or the basal cell layer. In some cases, the depth profile may extend at least 250 μm, 300μ, 350 μm, 400 μm, 450 μm, 500 μm, 550 μm, 600 μm, 650 μm, 700 μm, 750 μm, or farther below the surface of the tissue. In some cases, the depth profile may extend at most 750 μm, 700 μm, 650 μm, 600 μm, 550 μm, 500 μm, 450 μm, 400 μm, 350 μm, 300 μm, 250 μm, or less below the surface of the tissue. In some cases, the depth profile extends between about 100 μm and 1 mm, between about 200 μm and 900 μm, between about 300 μm and 800 μm, between about 400 μm and 700 μm, or between about 500 μm and 600 μm below the surface of the tissue.

The method may further comprise processing the depth profile using the one or more computer processors to identify a disease in the tissue. The identification of the disease in the tissue may comprise one or more characteristics. The one or more characteristics may provide a quantitative value or values indicative of one or more of the following: a likelihood of diagnostic accuracy, a likelihood of a presence of a disease in a subject, a likelihood of a subject developing a disease, a likelihood of success of a particular treatment, or any combination thereof. The one or more computer processors may also be configured to predict a risk or likelihood of developing a disease, confirm a diagnosis or a presence of a disease, monitor the progression of a disease, and monitor the efficacy of a treatment for a disease in a subject.

The method may further comprise contacting the tissue of the subject with the optical device. The contact may be direct or indirect contact. If the contact is a direct contact, performing the contact may comprise placing the optical device next to the tissue of the subject without an intervening layer. If the contact is an indirect contact, performing the contact may comprise placing the optical device next to the tissue of the subject with one or more intervening layers. The one or more intervening layers may comprise, but are not limited to, clothes, medical gauzes, and bandages. The contact may be monitored such that when contact between the surface of the epithelial tissue and the optical device is disrupted, a shutter positioned in front of the detector (e.g., relative to the path of light) can be activated and block incoming light.

According to some representative embodiments, the scanning pattern may follow a slanted plane. The slanted plane may be positioned along a direction that is angled with respect to an optical axis of the optical device. The angle between the slanted plane and the optical axis may be at most 45°. The angle between the slanted plane and the optical axis may be greater than or equal to about 5°, 10°, 15°, 20°, 25°, 30°, 35°, 40°, 45°, 55°, 60°, 65°, 70°, 75°, 80°, 85°, or greater. In other cases, the angle between the slanted plane and the optical axis may be less than or equal to about 85°, 80°, 75°, 70°, 65°, 60°, 55°, 50°, 45°, 35°, 30°, 25°, 20°, 15°, 10°, 5°, or less. In some cases, the angle between the slanted plane and the optical axis may be between any of the two values described above, for example, between about 5° and 50°.

According to various representative embodiments, the scanning path or pattern may follow one or more patterns that are designed to obtain enhanced, improved, or optimized image resolution. The scanning path or pattern may comprise, for example, one or more perpendicular planes, one or more slanted planes, one or more spiral focal paths, one or more zigzag or sinusoidal focal paths, or any combination thereof. The scanning path or pattern may be configured to maintain the scanning focal points near the optical element's center while moving in slanted directions. The scanning path or pattern may be configured to maintain the scanning focal points near the center of the optical axis (e.g., the focal axis).

The scanning pattern of the plurality of focal points may be selected by an algorithm. For example, a series of images may be obtained using focal points moving at one or more scan angles (with respect to the optical axis). The scanning pattern may include perpendicular scanning and/or slant scanning. Depending upon the quality of the images obtained, one or more additional images may be obtained using different scan angles or combinations thereof, selected by an algorithm. As an example, if an image obtained using a perpendicular scan or a smaller angle slant scan is of low quality, a computer algorithm may direct the system to obtain images using a combination of scan directions or using larger scan angles. If the combination of scan patterns results in an improved image quality, then the imaging session may continue using that combination of scan patterns. The method may be performed in an absence of removing the tissue from the subject. The method may be performed in an absence of administering a contrast enhancing agent to the subject.

The excitation light beam may comprise unpolarized light. In other embodiments, the excitation light beam may comprise polarized light. A wavelength of the excitation light beam can be at least about 400 nanometers (nm), 450 nm, 500 nm, 550 nm, 600 nm, 650 nm, 700 nm, 750 nm, 800 nm, 850 nm, 900 nm, 950 nm or longer. In some cases, a wavelength of the excitation light beam can be at most about 950 nanometers (nm), 900 nm, 850 nm, 800 nm, 750 nm, 700 nm, 650 nm, 600 nm, 550 nm, 500 nm, 450 nm, 400 nm or shorter. The wavelength of the pulses of light may be between about 700 nm and 900 nm, between about 725 nm and 875 nm, between about 750 nm and 850 nm, or between about 775 nm and 825 nm.

Multiple wavelengths may also be used. When multiple wavelengths of light are used, the wavelengths can be centered at least about 400 nm, 450 nm, 500 nm, 550 nm, 600 nm, 650 nm, 700 nm, 750 nm, 800 nm, 850 nm, 900 nm, 950 nm or longer with a bandwidth of at least about 10 nm, 20 nm, 30 nm, 40 nm, 50 nm, 75 nm, 100 nm, 125 nm, 150 nm, 175 nm, 200 nm, 225 nm, 250 nm, 275 nm, 300 nm or longer. For example, the wavelengths can be centered at about 780 nm with a bandwidth of about 50 nm (e.g., about ((780−(50/2))=755 nm) to about ((780+(50/2))=805 nm)). In some cases, the wavelengths can be centered at most about 950 nanometers (nm), 900 nm, 850 nm, 800 nm, 750 nm, 700 nm, 650 nm, 600 nm, 550 nm, 500 nm, 450 nm, 400 nm or shorter with a bandwidth of at least about 10 nm, 20 nm, 30 nm, 40 nm, 50 nm, 75 nm, 100 nm, 125 nm, 150 nm, 175 nm, 200 nm, 225 nm, 250 nm, 275 nm, 300 nm or longer.

The subset of the signals may comprise at least one of signal selected from the group consisting of second harmonic generation (SHG) signal, third harmonic generation (THG) signal, reflectance confocal microscopy (RCM) signal, and autofluorescence signal. SHG, THG, RCM, and autofluorescence are disclosed elsewhere herein. The subset of signals may comprise one or more generated signals as defined herein.

The collecting may be performed in a presence of ambient light. Ambient light can refer to normal room lighting, such as provided by various types of electric lighting sources including incandescent light bulbs or lamps, halogen lamps, gas-discharge lamps, fluorescent lamps, light-emitting diode (LED) lamps, and carbon arc lamps, in a medical examination room or an operating area where a surgical procedure is performed.

The simultaneously adjusting the depth and the position of the focal point of the excitation light beam along the slant scan, scan path or scan pattern may increase a maximum resolution depth of the depth profile. The maximum resolution depth after the increase may be at least about 1.1 times, 1.2 times, 1.5 times, 1.6 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, or greater of the original maximum resolution depth. In other embodiments, the maximum resolution depth after the increase may be at most about 3 times, 2.9 times, 2.8 times, 2.7 times, 2.6 times, 2.5 times, 2.4 times, 2.3 times, 2.2 times, 2.1 times, 2.0 times, 1.9 times, 1.8 times, 1.7 times, 1.6 times, 1.5 times, 1,4 times, or less of the original maximum resolution depth. The increase may be relative to instances in which the depth and the position of the focal point may be not simultaneously adjusted.

The signals indicative of the intrinsic property of the tissue may be detected by a photodetector. A power and gain of the photodetector sensor may be modulated to enhance image quality. The excitation light beam may be synchronized with sensing by the photodetector.

The RCM signals may be detected by a series of optical components in optical communication with a beam splitter. The beam splitter may be a polarization beam splitter, a fixed ratio beam splitter, a reflective beam splitter, or a dichroic beam splitter. The beam splitter may transmit greater than or equal to about 1%, 3%, 5%, 10%, 15%, 20%, 25%, 33%, 50%, 66%, 75%, 80%, 90%, 99% or more of incoming light. The beam splitter may transmit less than or equal to about 99%, 90%, 80%, 75%, 66%, 50%, 33%, 25%, 20%, 15%, 10%, 5%, 3%, 1%, or less of incoming light. The series of optical components may comprise one or more mirrors. The series of optical components may comprise one or more lenses. The fiber optic may be a single mode, a multi-mode, or a bundle of fiber optics.

The method may be performed without penetrating the tissue of the subject. Methods disclosed herein for identifying a disease in a tissue of a subject can be used during and/or for the treatment of the disease, for example during Mohs surgery to treat skin cancer. In some cases, identifying a disease, for example a skin cancer, in an epithelial tissue of a subject can be performed in the absence of removing the epithelial tissue from the subject. This may advantageously prevent pain and discomfort to the subject and can expedite detection and/or identification of the disease. The location of the disease may be detected in a non-invasive manner, which can enable a user such as a healthcare professional (e.g., surgeon, physician, nurse, or other practitioner) to determine the location and/or boundary of the diseased area prior to surgery. Identifying a disease in an epithelial tissue of a subject, in some cases, can be performed without penetrating the epithelial tissue of the subject, for example by a needle.

The disease or condition may comprise a cancer. In some cases, a cancer may comprise thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g., Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g., uterine sarcoma), vaginal cancer, vulvar cancer, or Waldenstrom's macroglobulinemia. The disease may be epithelial cancer. The epithelial cancer may be skin cancer.

The method may further comprise processing the depth profile using the one or more computer processors to classify a disease of the tissue. The classification may identify the tissue as having the disease at an accuracy, selectivity, and/or specificity of at least about 10%, 20%, 30%, 40%, 50%, 60%, 70%, 80%, 90%, 95%, 98%, 99%, 99.9%, or more. The classification may identify the tissue as having the disease at an accuracy, selectivity, and/or specificity of at most about 99.9%, 99%, 98%, 95%, 90%, 80%, 70%, 60%, 50%, 40%, 30%, 20%, 10%, or less. The one or more computer processors may classify the disease using one or more computer programs. The one or more computer programs may comprise one or more machine learning techniques. The one or more machine learning techniques may be trained on a system other than the one or more processors.

The depth profile may have a resolution of at least about 0.5, 0.6, 0.7, 0.8, 0.9, 1, 2, 3, 4, 5, 6, 7, 8, 9, 10, 15, 20, 25, 30, 40, 50, 75, 100, 150, 200 microns, or more. The depth profile may have a resolution of at most about 200, 150, 100, 75, 50, 40, 30, 25, 20, 15, 10, 9, 8, 7, 6, 5, 4, 3, 2, 1, 0.9, 0.8, 0.7, 0.6, 0.5 microns, or less. For example, the depth profile may be able to resolve an intercellular space of 1 micron.

In some embodiments the data is normalized with respect to illumination intensity.

The method may further comprise measuring a power of the excitation light beam. A power meter may be used to measure the power of the excitation light beam. The power meter may measure the power of the excitation light beam in real time. The one or more computer processors may normalize a signal for the measured power of the excitation light beam. The normalized signal may be normalized with respect to an average power, an instantaneous power (e.g., the power read at the same time as the signal), or a combination thereof. The one or more computer processors may generate a normalized depth profile. The normalized depth profile may be able to be compared across depth profiles generated at different times. The depth profile may also include information related to the illumination power at the time the image was obtained. A power meter may also be referred to herein as a power sensor or a power monitor.

The method may allow for synchronized collection of a plurality of signals. The method may enable collection of a plurality of signals generated by a single excitation event. A depth profile can be generated using signals, as described elsewhere herein, that are generated from the same excitation event. A user may decide which signals to use to generate a depth profile.

The method may generate two or more layers of information. The two or more layers of information may be information generated from data generated from the same light pulse of the single probe system. The two or more layers may be from a same depth profile. Each of the two or more layers may also form separate depth profiles from which a projected cross section image may be created or displayed. For example, each separate layer, or each separate depth profile may correspond to a particular processed signal or signals that correspond to a particular imaging method. For example, a depth profile can be generated by taking confocal microscopy signals from skin tissue, two-photon fluorescence signals from melanin and another depth profile can be generated using SHG signals from collagen, and three or more depth profiles can be overlaid as multiple layers of information. Each group of signals can be separately filtered, processed, and used to create individual depth profiles and projected cross section images, combined into a single depth profile with data that can be used to generate a projected cross section image, data from each group of signals can be combined and the combination can be used to generate a single depth profile, or any combination thereof. Each group of signals that correspond to a particular feature or features of the tissue can be assigned a color used to display the individual cross section images of the feature or features or a composite cross section image including data from each group of signals. The cross-sectional images or individual depth profiles can be overlaid to produce a composite image or depth profile. Thus, a multi-color, multi-layer, depth profile or image can be generated.

### Example Images

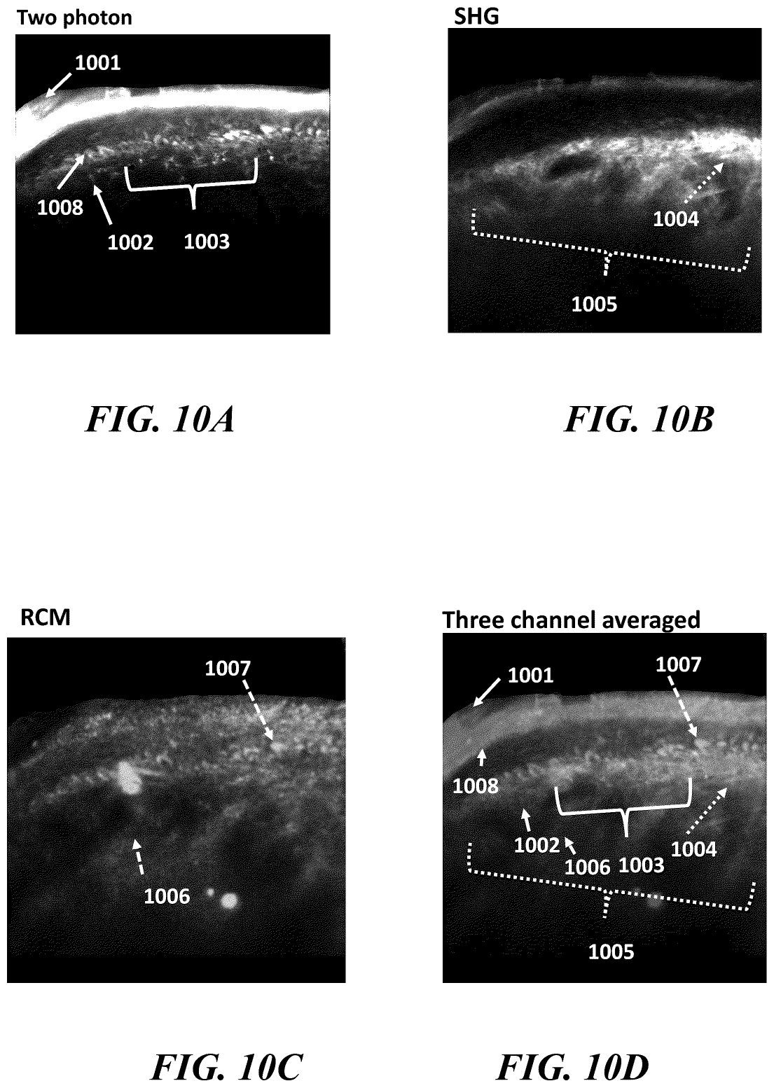

FIGS. 10A-10D illustrate an example of images formed from depth profiles in skin. FIG. 10A illustrates an image displayed from a depth profile derived from a generated signal resulting from two-photon autofluorescence. The autofluorescence signal was generated from an excitation signal of about 780 nm and was collected into a light guide from a probe at the tip of the optical device. The autofluorescence signal was detected over a range of about 415 to 650 nm with an appropriately selected optical filter. The epidermis 1003 can be seen along with the stratum corneum layer 1001 at the surface of the skin. Elastin 1002 at the boundary of epidermis 1003 and dermis 1005 layers can be seen as well as epithelial cells 1008 (keratinocytes) in the epidermis 1003 along with other features. FIG. 10B illustrates an image displayed from a depth profile or layer that is synchronized in time and location with the depth profile or layer of FIG. 10A. The image displayed from the depth profile in FIG. 10B is derived from a second harmonic generation signal at about 390 nm detected with an appropriately selected optical filter. The second harmonic generation signal was generated from an excitation signal of about 780 nm and was collected into a light guide from a probe at the tip of the optical device. Collagen 1004 in the dermis layer 1005 can be seen as well as other features. FIG. 10C illustrates an image displayed from a depth profile or layer that is synchronized in time and location with the depth profiles or layers of FIGS. 10A and 10B. The image displayed from the depth profile in FIG. 10C is derived from a reflectance confocal signal reflected back to an RCM detector. The reflected signal of about 780 nm was directed back through its path of origin and split to an alignment arrangement that focused and aligned the reflected signal into an optical fiber for detection and processing. Melanocytes 1007 and collagen 1006 can be seen as well as other features. The images in FIGS. 10A, 10B and 10C can be derived from a single composite depth profile resulting from the excitation light pulses and having multiple layers or can be derived as single layers from separate depth profiles. FIG. 10D shows overlaid images of FIGS. 10A-10C. The boundaries that can be identified from the features of FIGS. 10A and 10B can help identify the location of the melanocyte identified in FIG. 10D. Diagnostic information can be contained in the individual images and/or the composite or overlaid image of FIG. 10D. For example, it is believed that some suspected lesions can be identified based on the location and shape of the melanocytes or keratinocytes in the various tissue layers. The depth profiles of FIGS. 10A-10D may be examples of data for use in a machine learning algorithm as described. For example, all three layers can be input into a machine learning classifier as individual layers, as well as using the composite image as another input.

### Optical Techniques for Detecting Epithelial Cancers

The present disclosure provides optical techniques that may be used for diagnosing epithelial diseases and skin pathologies. Optical imaging techniques can display nuclear and cellular morphology and may offer the capability of real-time detection of tumors in large areas of freshly excised or biopsied tissue without sample processing, such as that of histology. Optical imaging methods can also facilitate non-invasive, real-time visualization of suspicious tissue without excising, sectioning, and/or staining the tissue sample. Optical imaging may improve the yield of diagnosable tissue (e.g., by avoiding areas with fibrosis or necrosis), minimize unnecessary biopsies or endoscopic resections (e.g., by distinguishing neoplastic from inflammatory lesions), and assess surgical margins in real-time to confirm negative margins (e.g., for performing limited resections). The ability to assess a tissue sample in real-time, without needing to wait for tissue processing, sectioning, and staining, may improve diagnostic turnaround time, especially in time-sensitive contexts, such as during Mohs surgery. Non-limiting examples of optical imaging techniques for diagnosing epithelial diseases and cancers include multiphoton microscopy, autofluorescence microscopy, polarized light microscopy, confocal microscopy, Raman spectroscopy, optical coherence tomography, and ultrasonography. Non-limiting examples of detectable tissue components include keratin, NADPH, melanin, elastin, flavins, protoporphyrin ix, and collagen. Other detectable components can include tissue boundaries. Example images from depth profiles shown in FIGS. 10A-10D show some detectable components, such as, for example, including but not limited to tissue boundaries for stratum corneum, epidermis, and dermis, melanocytes, collagen, and elastin.

Confocal microscopy may be used to examine epithelial tissue. Exogenous contrast agents may be administered for enhanced visibility. Confocal microscopy can provide non-invasive images of nuclear and cellular morphology in about 2-5 μm thin sections in living human skin with lateral resolution of about 0.5-1.0 μm. Confocal microscopy can be used to visualize in vivo micro-anatomic structures, such as the epidermis, and individual cells, including melanocytes.

Multiphoton microscopy (MPM) can be used to image intrinsic molecular signals in living imaging subjects, such as the skin tissue of a patient. In MPM, a sample may be illuminated with light at wavelengths longer than the normal excitation wavelength, for example twice as long or three times as long. MPM can include second harmonic generation microscopy (SHG) and third harmonic generation microscopy (THG). Third harmonic generation may be used to image nerve tissue.

Autofluorescence microscopy can be used to image biological molecules (e.g. fluorophores) that are inherently fluorescent. Non-limiting examples of endogenous biological molecules that are autofluorescent include nicotinamide adenine dinucleotide (NADH), NAD(P)H, flavin adenine dinucleotide (FAD), collagen, retinol, and tryptophan and the indoleamine derivatives of tryptophan. Changes in the fluorescence level of these fluorophores, such as with tumor progression, can be detected optically. Changes may be associated with altered cellular metabolic pathways (NADH, FAD) or altered structural tissue matrix (collagen).

Polarized light can be used to evaluate biological structures and examine parameters such as cell size and refractive index. Refractive index can provide information regarding the composition and organizational structure of cells, for example cells in a tissue sample. Cancer can significantly alter tissue organization, and these changes may be detected optically with polarized light.

Raman spectroscopy may also be used to examine epithelial tissue. Raman spectroscopy may rely on the inelastic scattering (so-called “Raman” scattering) phenomena to detect spectral signatures of disease progression biomarkers such as lipids, proteins, and amino acids.

Optical coherence tomography may also be used to examine epithelial tissue. Optical coherence tomography may be based on interferometry in which a laser light beam is split with a beam splitter, sending some of the light to the sample and some of the light to a reference. The combination of reflected light from the sample and the reference can result in an interference pattern which can be used to determine a reflectivity profile providing information about the spatial dimensions and location of structures within the sample. Current, commercial optical coherence tomography systems have lateral resolutions of about 10 to 15 μm, with depth of imaging of about 1 mm or more. Although this technique can rapidly generate 3-dimensional (3D) image volumes that reflect different layers of tissue components (e.g., cells, connective tissue, etc.), the image resolution (e.g., similar to the ×4 objective of a histology microscope) may not be sufficient for routine histopathologic diagnoses.

Ultrasound may also be used to examine epithelial tissue. Ultrasound can be used to assess relevant characteristics of epithelial cancer such as depth and vascularity. While ultrasonography may be limited in detecting pigments such as melanin, it can supplement histological analysis and provide additional detail to assist with treatment decisions. It may be used for noninvasive assessment of characteristics, such as thickness and blood flow, of the primary tumor and may contribute to the modification of critical management decisions.

Methods for diagnosing epithelial diseases and skin pathologies disclosed herein may comprise one or more of multiphoton microscopy, autofluorescence microscopy, polarized light microscopy, confocal microscopy, Raman spectroscopy, optical coherence tomography, and ultrasonography. In some cases, a method for diagnosing an epithelial disease and/or skin pathology comprises autofluorescence microscopy and multiphoton microscopy. As an alternative, a method for diagnosing an epithelial disease and/or skin pathology comprises autofluorescence microscopy, multiphoton microscopy, and polarized light microscopy. Both second harmonic generation microscopy and third harmonic generation microscopy can be used. In some cases, one of second harmonic generation microscopy and third harmonic generation microscopy is used.

Methods for diagnosing epithelial diseases and skin pathologies disclosed herein may comprise using one or more depth profiles to identify anatomical features and/or other tissue properties or characteristics and overlaying the images from the one or more depth profiles to an image from which a skin pathology can be identified.

### Computer Systems

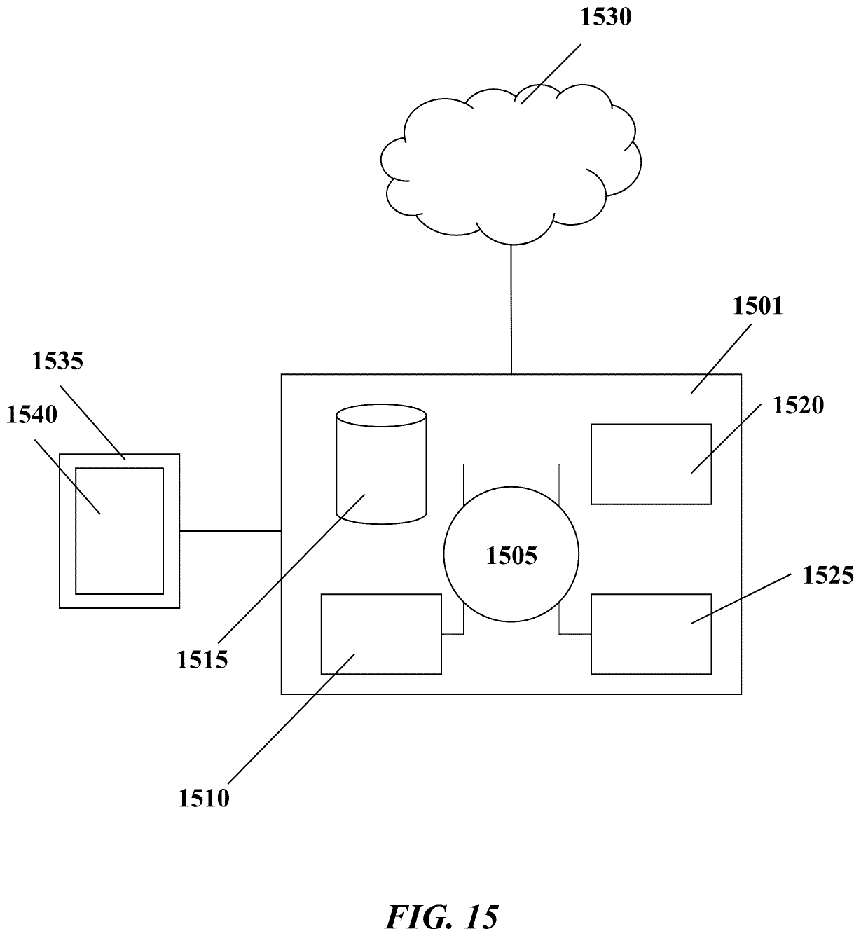

The present disclosure provides computer systems that are programmed to implement methods of the disclosure. FIG. 15 shows a computer system 1501 that is programmed or otherwise configured to direct the systems and devices described elsewhere herein to direct light to an imaging subject and collect light from the imaging subject. The computer system 1501 may further be configured or otherwise programmed to use the light from the imaging subject to image the imaging subject. The computer system 1501 can regulate various aspects of the systems and methods of the present disclosure, such as, for example, directing light to the imaging subject, focusing the light at various locations on or depths in the imaging subject, and generating images of the imaging subject. The computer system 1501 may be a part of a mobile or portable imaging system. Alternatively, or in addition to, the computer system 1501 may be a remote device wirelessly connected to the imaging system.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1130. For instance, the computer system 1501 can communicate with a remote computer system of a user (e.g., laptop, cellular phone, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, control of settings for imaging the imaging subject and images of the imaging subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, be configured to image the imaging subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-20. (canceled)

21. A device, comprising:
a probe configured to (i) direct a beam of light from a light source to an imaging subject and (ii) collect light from said imaging subject upon said beam of light contacting said imaging subject; and
a light filtering device in optical communication with said probe, wherein said light filtering device comprises (a) a chamber having (i) an input configured to receive said light collected by said probe and (ii) an output configured to transmit a focused portion of said light away from said chamber and (b) a plurality of reflectors disposed in said chamber, wherein said plurality of reflectors is configured to direct said light received, in an optical path from said input to said output via reflectance of said light between reflectors of said plurality of reflectors, wherein said chamber is configured to reject out of focus light along said optical path.

22. The device of claim 21, wherein a cross sectional diameter of said output is greater than or equal to about 0.1 times a cross sectional diameter of said input.

23. The device of claim 21, wherein said chamber has a longest internal linear dimension, and wherein said optical path has a path length that is at least 3 times a length of said longest linear dimension.

24. The device of claim 21, wherein said optical path has a pathlength greater than or equal to 0.25 meters, 0.5 meters, or 1 meter.

25. The device of claim 21, wherein at least one reflector of said plurality of reflectors is a retroreflector.

26. The device of claim 21, wherein said output is configured to direct a focused portion of said light to a collector wherein said device comprises said collector.

27. The device of claim 21, wherein said plurality of reflectors includes a first reflector, wherein said plurality of reflectors are arranged to direct a focused portion of the light in an optical path between the reflectors, and wherein the optical path returns to the first reflector a plurality of times before reaching the output.

28. The device of claim 21, wherein said plurality of reflectors comprises a first reflector at a first location within said chamber and a second reflector at a second location within said chamber, which first reflector is configured to direct said light to said second reflector, and said optical path has a path length, wherein said first reflector and said second reflector are separated by a distance, and wherein said pathlength is at least five times said distance.

29. The device of claim 28, wherein said pathlength that said light traverses is from five times to thirty times said distance.

30. The device of claim 21, further comprising a scanning unit disposed between said light source and said probe, wherein said scanning unit is configured to scan said beam of light across said imaging subject.

31. The device of claim 21, wherein said probe comprises an objective configured to collimate said light.

32. The device of claim 21, further comprising an alignment unit disposed between said probe and said light filtering device, wherein said alignment unit is configured to (i) direct said light to said light filtering device and (ii) adjust an angle of entry of said light into said chamber.

33. The device of claim 21, further comprising a beam splitter disposed between said probe and said light filtering device, wherein said beam splitter is configured to (i) split said light to generate a split light and (ii) direct at least a portion of said split light to said light filtering unit.

34. The device of claim 21, wherein said device is configured for confocal imaging.

35. The device of claim 21, further comprising a beam de-expander unit positioned between said beam of light and said light filtering device.

36. A method for imaging an imaging subject, comprising:
(a) providing a probe in optical communication with (i) a light filtering device comprising a chamber and (ii) said imaging subject;
(b) transmitting said beam of light from a light source to an imaging subject via said probe and collecting light from said imaging subject upon said beam of light contacting said imaging subject.
(c) using said probe to provide a beam of light to said imaging subject and collect a resulting return beam of light from said imaging subject;
(d) directing said return beam of light from said imaging subject to said chamber of said light filtering device;
(e) repeatedly directing said beam of light from a first reflector to a second reflector within said chamber of said light filtering device such that a pathlength that said beam of light traverses between said first reflector and said second reflector is at least three times a distance separating said first reflector and said second reflector; and
(f) processing said beam of light to generate an image of said imaging subject.

37. The method of claim 36, further comprising, prior to (d), directing said return beam of light to a beam de-expander unit.

38. The method of claim 37, further comprising, subsequent to (d), processing said light to generate an image of said imaging subject.

39. The method of claim 37, further comprising using said light for confocal imaging.

40. The method of claim 36, wherein said pathlength said beam of light traverses is at least five times said distance separating said first location and said second location.
